(19) United States
(12) Patent Application Publication
(10) Pub. No.: US 2021/0127651 A1
(43) Pub. Date: May 6, 2021

(54) GENE DRIVE TARGETING FEMALE DOUBLESEX SPLICING IN ARTHROPODS

(71) Applicant: IMPERIAL COLLEGE OF SCIENCE, TECHNOLOGY AND MEDICINE, London (GB)

(72) Inventors: Andrea Crisanti, London (US); Kyros Kyroi, London (GB); Andrew Hammond, London (GB)

(73) Assignee: IMPERIAL COLLEGE OF SCIENCE, TECHNOLOGY AND MEDICINE, London (GB)

(21) Appl. No.: 17/253,553

(22) PCT Filed: Jun. 21, 2019
(86) PCT No.: PCT/GB2019/051757

§ 371 Date: Dec. 17, 2020

(30) Foreign Application Priority Data
Jun. 22, 2018 (GB) 1810253.3

(51) Int. Cl. A01K 67/033; C12N 15/85

(52) U.S. Cl. CPC A01K 67/0339; C12N 15/8509; A01K 2267/02; A01K 2217/07; A01K 2227/706

(57) ABSTRACT

The invention relates to gene drives, and in particular to genetic sequences and constructs for use in a gene drive. The invention is especially concerned with ultra-conserved and ultra-constrained sequences for use as a gene drive target with the aim of overcoming the development of resistance to the drive. The invention is also concerned with methods of suppressing wild type arthropod populations by use of the gene drive construct described herein.

The invention relates to gene drives, and in particular to genetic sequences and constructs for use in a gene drive. The invention is especially concerned with ultra-conserved and ultra-constrained sequences for use as a gene drive target with the aim of overcoming the development of resistance to the drive. The invention is also concerned with methods of suppressing wild type arthropod populations by use of the gene drive construct described herein.

A gene drive is a genetic engineering approach that can propagate a particular suite of genes throughout a target population. Gene drives have been proposed to provide a powerful and effective means of genetically modifying specific populations and even entire species. For example, applications of gene drive include either suppressing or eliminating insects that carry pathogens (e.g. mosquitoes that transmit malaria, dengue and zika pathogens), controlling invasive species, or eliminating herbicide or pesticide resistance.

CRISPR-CAS9 nucleases have recently been employed in gene drive systems to target endogenous sequences of the human malaria vector Anopheles gambiae and Anopheles stephensi with the objective to develop genetic vector control measures^{1,2}. These initial proof-of-principle experiments have demonstrated the potential of gene drive approaches and translated a theoretical hypothesis into a powerful genetic tool potentially capable of modifying the genetic makeup of a species and changing its evolutionary destiny either by suppressing its reproductive capability or permanently modifying the outcome of the mosquito interaction with the malaria parasites they transmit.

According to mathematical modelling, suppression of A. gambiae mosquito reproductive capability can be achieved using gene drive systems targeting haplosufficient female fertility genes^{3,4}, or alternatively by introducing into the Y chromosome a sex distorter in the form of a nuclease designed to shred the X chromosome during meiosis, an approach known as Y-drive⁴⁻⁶. Both strategies are anticipated to cause a progressive decrease of the number of fertile females to the point of population collapse. However, a number of technical and scientific issues need to be addressed in order to progress from proof-of-principle demonstration to the availability of an effective gene drive system for vector population suppression. The development of a Y-drive has so far proven difficult because of the complete transcriptional shut down of the sex chromosomes during meiosis that prevents the expression of a Y-linked sex distorter during gamete formation^{6,7}.

A gene drive system designed to destroy the A. gambiae fertility gene AGAP007280, after an initial increase in frequency, induced in the span of a few subsequent generations the selection of nuclease-resistant functional variants that completely blocked the spread of the drive². These variants comprised small insertions or deletions (i.e. indels) of differing length generated by non-homologous end joining repair following nuclease activity at the target site. The development of resistance to the gene has been largely predicted³ and is regarded as the main technical obstacle for the development of an effective gene drive for vector controls⁸⁻¹¹.

As described in the Examples, the inventors have developed novel genetic constructs for use in a gene drive approach which targets a key sequence of the doublesex gene of Anopheles gambiae essential for the maturation of female specific transcript of this gene. The doublesex gene has been shown to be ultra-conserved and ultra-constrained, and so represents a robust target gene for a gene drive approach.

Accordingly, in a first aspect of the invention, there is provided a gene drive genetic construct capable of disrupting an intron-exon boundary of the female specific splice form of the doublesex (dsx) gene in an arthropod, such that when the construct is expressed, the intron-exon boundary is disrupted and at least one exon is spliced out of a doublesex precursor-mRNA transcript, wherein a female arthropod, which is homozygous for the construct, exhibits a suppressed reproductive capacity.

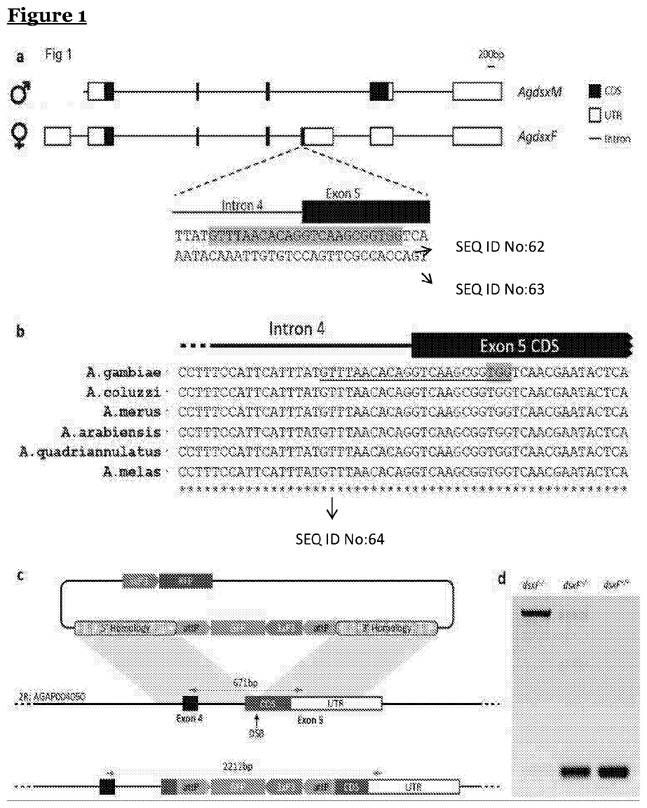

Sex differentiation in insect species follows a common pattern where a primary signal activates a key gene that in turn induces a cascade of molecular events that ultimately control the alternative splicing of the gene doublesex (dsx)^{12,13}. With the exception of Yob1 acting as Y-linked male determining factor¹⁴, the molecular mechanisms and the genes involved in regulating sex differentiation in A. gambiae are not well understood. However, without wishing to be bound to any particular theory, the inventors hypothesise that the gene dsx is key in determining the sexual dimorphism in this mosquito species¹⁵. In A. gambiae, dsx (i.e. Agdsx) consists of seven exons, distributed over an 85-kb region on chromosome 2R, with similarities in gene structure to D. melanogaster dsx (Dmdsx) and orthologues from other insects, and is alternatively spliced in the two sexes to produce the female and male transcripts AgdsxF and AgdsxM, respectively. The female transcript consists of a 5′ segment common with males, a highly conserved female-specific exon (exon 5) and a 3′ common region, while the male transcript comprises only the 5′ and 3′ common segments. The male-specific region is transcribed as non-coding 3′ UTR in females, as shown in FIG. 1a.

The inventors have surprisingly identified that this female-specific exon (i.e. exon 5) of dsx is ultra-conserved across the Anopheles gambiae species complex and even throughout the wider Anophelinae subfamily, as shown in FIGS. 1b and 11a, and 12. This type of ultra-conservation is very rare because even proteins that are highly constrained show some variation at the level of the DNA sequence because “silent” variation does not alter the composition of the final encoded protein. The inventors carefully assessed the ultra-conserved sequence in the doublesex gene and, without wishing to be bound to any particular theory, believe that it is the splice acceptor site at the 5′ boundary of exon 5 that is required for sex-specific splicing of dsx into the female form, as this sequence may represent the target of RNA binding proteins that direct the alternative splicing of this important exon.

The inventors were especially surprised to observe that targeting an intron-exon boundary of the female specific splice form of the doublesex (dsx) gene resulted in suppressed reproductive capacity in females which were homozygous for the construct. This was because their previous studies had strongly suggested that intron 4 was spliced mainly in males, as indicated by a fluorescent reporter construct designed to be activated by the splicing of intron 4.

The inventors generated the gene drive construct of the first aspect such that it targets the splice acceptor site at the 5′ boundary of exon 5 of dsx, and were surprised to observe that, in stark contrast to all previous demonstrations of gene drive, no resistance was selected after release into caged populations of the mosquito. Moreover, additional experiments that were designed to reveal rare instances of resistance that were not selected in caged experiments also surprisingly failed to detect putative resistant mutations, thereby indicating that all mutations that were generated did not restore dsx function. The inventors have demonstrated that disruption of a female-specific exon (exon 5) of dsx leads to incomplete sexual dimorphism in females, but not males. When female mosquitoes carry this mutation in homozygosity, they display a range of mutant attributes including the inability to produce ovaries and biting mouthparts—an advantageous outcome that is optimally suited for a gene drive aimed at population suppression.

The inventors have therefore demonstrated that the gene drive construct of the invention can be used to spread through, replace and ultimately suppress any arthropod population by using the ultra-conserved, ultra-constrained sites found in different species at the intron/exon boundary of the female specific exon. The development of the gene drive construct of the invention which is capable of collapsing a human malaria vector population is a long sought scientific and technical achievement. The inventors describe herein a gene drive solution that shows a number of desired efficacy features for field applications in term of inheritance bias, fertility of heterozygous carrier individuals, phenotype of homozygous females and lack of nuclease-resistant functional variants at the target site. Advantageously, these results open a new phase in the effort to develop novel vector control measures and will stimulate unprecedented interest in the scientific community as well as among both policy makers and the general public.

Furthermore, the inventors believe that the results disclosed herein will have implications well beyond the field of malaria vector control, i.e. A. gambiae. The highly conserved functional role of dsx for sex determination in all insect species so far analysed and the high degree of sequence conservation amongst members of the same species in regions involved in sex specific splicing suggests that these sequences represent an Achilles heel for similar gene drive solutions aimed at targeting other vector species and agricultural pests.

It will be appreciated that suppression of a female's reproductive capacity can relate to a reduced ability of the female of the specific to procreate, or complete sterility of the female. Preferably, the reproductive capacity of the female homozygous for the construct is reduced by at least 5%, 10%, 20% or 30% compared to the corresponding wild type female. More preferably, the reproductive capacity of the female homozygous for the construct is reduced by at least 40%, 50% or 60% compared to the corresponding wild type female. Most preferably, the reproductive capacity of the female homozygous for the construct is reduced by at least 70%, 80%, 90% or 95% compared to the corresponding wild type female. Most preferably, suppression of a female's reproductive results in complete sterility of the female.

The skilled person will appreciate that the gene drive construct of the invention may relate to a construct comprising one or more genetic elements that biases its inheritance above that of Mendelian genetics, and thus increases in its frequency within a population over a number of generations.

Suitable arthropods which may be targeted using the gene drive genetic construct of the invention include insects, arachnids, myriapods or crustaceans. Preferably, the arthropod is an insect. Preferably, the arthropod, and most preferably the insect, is a disease-carrying vector or pest (e.g. agricultural pest), which can infect, cause harm to, or kill, an animal or plant of agricultural value, for example, Anopheline species, Aedes species (as a disease vector), Ceratitis capitata, or Drosophila species (as an agricultural pest).

Preferably, the insect is a mosquito. Preferably, the mosquito is of the subfamily Anophelinae. Preferably, the mosquito is selected from a group consisting of: Anopheles gambiae; Anopheles coluzzi; Anopheles merus; Anopheles arabiensis; Anopheles quadriannulatus; Anopheles stephensi; Anopheles arabiensis; Anopheles funestus; and Anopheles melas.

Most preferably, the mosquito is Anopheles gambiae.

The sequence of the doublesex gene in various arthropods, insects, and mosquito species are publicly available and so known to the skilled person. However, in a preferred embodiment, the doublesex gene is from Anopheles gambiae (referred to as AGAP004050), which is provided herein as SEQ ID No: 1. SEQ ID No:1 is the whole AGAP004050 gene, plus about 3000 bp upstream of its putative promter and about 4000 bp downstream of its putative terminator.

Accordingly, preferably the doublesex gene comprises a nucleic acid sequence substantially as set out in SEQ ID NO: 1, or a fragment or variant thereof.

Preferably, however, the intron-exon boundary targeted by the genetic construct of the invention is the boundary between intron 4 and exon 5 of the doublesex gene. In an embodiment, the intron 4-exon 5 boundary of the doublesex gene is provided herein as SEQ ID No: 2, as follows:

[SEQ ID No: 2]
CCTTTCCATTCATTTATGTTTAACACAGGTCAAGCGGTGGTCAACGAAT
ACTCACGATTGCATAATCTGAACATGTTTGATGGCGTGGAGTTGCGCAA
TACCACCCGTCAGAGTGGATGATAAACTTTC

Accordingly, preferably genetic construct targets a nucleic acid sequence comprising or consisting of the nucleotide sequence substantially as set out in SEQ ID NO: 2, or a fragment or variant thereof. In some embodiments, the genetic construct targets a nucleic acid sequence comprising or consisting of the nucleotide sequence substantially as set out in SEQ ID NO: 2, or a fragment or variant thereof. The target sequence may include up to 1, 2, 3, 4, 5, 10 or 15 nucleotides 5′ and/or 3′ of SEQ ID No:2.

In a preferred embodiment, the intron 4-exon 5 boundary of the doublesex gene targeted by the gene drive construct is provided herein as SEQ ID No: 3, as follows:

[SEQ ID No: 3]
CCTTTCCATTCATTTATGTTTAACACAGGTCAAGCGGTGGTCAACGAAT
ACTCA

Accordingly, preferably the genetic construct targets a nucleic acid sequence comprising or consisting of the nucleotide sequence substantially as set out in SEQ ID NO: 3, or a fragment or variant thereof. In some embodiments, the genetic construct targets a nucleic acid sequence comprising or consisting of the nucleotide sequence substantially as set out in SEQ ID NO: 3, or a fragment or variant thereof. The target sequence may include up to 1, 2, 3, 4, 5, 10 or 15 nucleotides 5′ and/or 3′ of SEQ ID No:3.

In a most preferred embodiment, the intron 4-exon 5 boundary of the doublesex gene targeted by the gene drive construct is provided herein as SEQ ID No: 4, as follows:

[SEQ ID No: 4]
GTTTAACACAGGTCAAGCGGTGG

Accordingly, most preferably the genetic construct targets a nucleic acid sequence comprising or consisting of the nucleotide sequence substantially as set out in SEQ ID NO: 4, or a fragment or variant thereof. In some embodiments, the genetic construct targets a nucleic acid sequence comprising or consisting of the nucleotide sequence substantially as set out in SEQ ID NO: 4, or a fragment or variant thereof. The target sequence may include up to 1, 2, 3, 4, 5, 10 or 15 nucleotides 5′ and/or 3′ of SEQ ID No:4.

The concept of gene drive genetic constructs is known to those skilled in the art. Preferably, the gene drive genetic construct is a nuclease-based genetic construct. The gene drive genetic construct may be selected from a group consisting of: a transcription activator-like effector nuclease (TALEN) genetic construct; Zinc finger nuclease (ZFN) genetic construct; and a CRISPR-based gene drive genetic construct. Preferably, the genetic construct is a CRISPR-based gene drive construct, most preferably a CRISPR-Cpf1-based or CRISPR-Cas9-based gene drive genetic construct. However, it will be appreciated that other nucleases used in CRISPR-based genomic engineering methods are know and may be used in accordance with the invention.

Accordingly, in an embodiment in which the genetic construct is a CRISPR-based gene drive genetic construct, the genetic construct comprises a first nucleotide sequence encoding a nucleotide sequence that is capable of hybridising to the intron-exon boundary of the doublesex (dsx) gene, preferably with the objective to disrupt or destroy the female specific splice form. Preferably, the nucleotide sequence encoded by the first nucleotide sequence which is capable of hybridising to the intron-exon boundary of the doublesex (dsx) gene is a guide RNA. Preferably, the guide RNA is at least 16 base pairs in length. Preferably, the guide RNA is between 16 and 30 base pairs in length, more preferably between 18 and 25 base pairs in length.

Preferably, the CRISPR-based gene drive genetic construct further comprises a second nucleotide sequence encoding a CRISPR nuclease, preferably a Cpf1 or Cas9 nuclease, and most preferably a Cas9 nuclease. The sequences of the CRISPR nuclease and encoding nucleotides are known in the art. The first and second nucleotide sequences may be on separate nucleic acid molecules forming two genetic constructs, which act in tandem (i.e. in trans) as the gene drive genetic construct of the invention. Preferably, however, the first and second nucleotide sequences are on, or form part of, the same nucleic acid molecule, thereby creating the gene drive genetic construct of the invention. Preferably, the second nucleotide sequence encoding the nuclease is disposed 5′ of the first nucleotide sequence encoding a nucleotide sequence that is capable of hybridising to the intron-exon boundary of the doublesex (dsx) gene.

In a preferred embodiment, the first nucleotide sequence encoding a nucleotide sequence that is capable of hybridising to the intron-exon boundary of the doublesex (dsx) gene (i.e. a guide RNA component) is provided herein as SEQ ID No: 5, as follows:

[SEQ ID No: 5]
GTTTAACACAGGTCAAGCGG

Accordingly, preferably the first nucleotide sequence encoding a nucleotide sequence that is capable of hybridising to the intron-exon boundary of the doublesex (dsx) gene comprises a nucleic acid sequence substantially as set out in SEQ ID NO: 5, or a fragment or variant thereof.

The part of the nucleotide sequence that is capable of hybridising to the intron-exon boundary (i.e. the guide RNA) is known as a protospacer. In order for the nuclease to function, it also requires a specific protospacer adjacent motif (PAM) that varies depending on the bacterial species of the nuclease encoding gene. The most commonly used Cas9 nuclease recognizes a PAM sequence of NGG that is found directly downstream of the target sequence in the genomic DNA on the non-target strand. Recognition of the PAM by the nuclease is believed to destabilise the adjacent sequence, allowing interrogation of the sequence by the guide RNA, and resulting in RNA-DNA pairing when a matching sequence is present. The PAM is not present in the guide RNA sequence, but needs to be immediately downstream of the target site in the genomic DNA.

The skilled person would understand that the nucleotide sequence (i.e. guide RNA) that is capable of hybridising to the intron-exon boundary of the doublesex (dsx) gene may further comprise a CRISPR nuclease binding sequence, preferably a Cpf1 or Cas9 nuclease binding sequence, and most preferably a Cas9 nuclease binding sequence. The CRISPR nuclease binding sequence creates a secondary binding structure which complexes with the nuclease, for example a hairpin loop. The PAM on the host genome is recognised by the nuclease.

Accordingly, in a preferred embodiment, the first nucleotide sequence encoding a nucleotide sequence that is capable of hybridising to the intron-exon boundary of the doublesex (dsx) gene (i.e. a guide RNA) is provided herein as SEQ ID No: 6, as follows:

[SEQ ID No: 6]
GTTTAACACAGGTCAAGCGGGTTTTAGAGCTAGAAATAGCAAGTTAAAA
TAAGGCTAGTCCGTTATCAACTTGAAAAAGTGGCACCGAGTCGGTGCT

Accordingly, preferably the first nucleotide sequence encoding a nucleotide sequence that is capable of hybridising to the intron-exon boundary of the doublesex (dsx) gene comprises or consists of a nucleic acid sequence substantially as set out in SEQ ID NO: 6, or a fragment or variant thereof. The underlined sequence denotes the spacer, which encodes the nucleotide which hybridises to the dsx target site (i.e. SEQ ID No:5), and the rest if the gRNA backbone necessary for complexing with the nuclease, i.e. it encodes the CRISPR nuclease binding sequence.

In one embodiment, the nucleotide sequence which is encoded by the first nucleotide sequence and which is capable of hybridising to the intron-exon boundary of the doublesex (dsx) gene (i.e. a guide RNA component) is provided herein as SEQ ID No: 58, as follows:

[SEQ ID No: 58]
GUUUAACACAGGUCAAGCGG

Accordingly, preferably the nucleotide sequence which is encoded by the first nucleotide sequence and which is capable of hybridising to the intron-exon boundary of the doublesex (dsx) gene (i.e. a guide RNA) comprises a nucleic acid sequence substantially as set out in SEQ ID NO: 58, or a fragment or variant thereof.

In one embodiment, the nucleotide sequence which is encoded by the first nucleotide sequence and which is capable of hybridising to the intron-exon boundary of the doublesex (dsx) gene (i.e. a guide RNA) is provided herein as SEQ ID No: 48, as follows:

[SEQ ID No: 48]
GUUUAACACAGGUCAAGCGGGUUUUAGAGCUAGAAAUAGCAAGUUAAAAU
AAGGCUAGUCCGUUAUCAACUUGAAAAAGUGGCACCGAGUCGGUGCU

Accordingly, preferably the nucleotide sequence which is encoded by the first nucleotide sequence and which is capable of hybridising to the intron-exon boundary of the doublesex (dsx) gene (i.e. a guide RNA) comprises or consists of a nucleic acid sequence substantially as set out in SEQ ID NO: 48, or a fragment or variant thereof.

The CRISPR-based gene drive genetic construct further comprises at least one promoter sequence, which drives expression of the first and second nucleotide sequence. In other words, expression of the first and second nucleotide sequences is under the control of the same promoter. Preferably, however, the CRISPR-based gene drive genetic construct comprises at least two promoter sequences, such that expression of the first and second nucleotide sequence is under the control of separate promoters. Preferably, therefore, the construct comprises a first promoter sequence operably linked to the first nucleotide sequence and a second promoter sequence operably linked to the second nucleotide sequence. The first and second promoter sequence may be any promoter sequence that is suitable for expression in an arthropod, and which would be known to those skilled in the art. Accordingly, the guide RNA is preferably expressed under control of the first promoter, and the nuclease is expressed under control of the second promoter.

Preferably, the first promoter is a polymerase III promoter, and most preferably a polymerase III promoter which does not add a 5′cap or a 3′polyA tail. More preferably, the promoter is a U6 promoter.

One embodiment of a nucleotide sequence of a U6 promoter is provided herein as SEQ ID No: 49, as follows:

[SEQ ID No: 49]
TTTGTATGCGTGCGCTTGAAGGGTTGATCGGAACCTTACAACAGTTGTAG
CTATACGGCTGCGTGTGGCTTCTAACGTTATCCATCGCTAGAAGTGAAAC
GAATGTGCGTAGGTATATATATGAAATGGAGTTGCTCTCTGCT

Accordingly, preferably the first promoter sequence comprises or consists of a nucleic acid sequence substantially as set out in SEQ ID No: 49, or a variant or fragment thereof.

Preferably, the second promoter sequence is a promoter sequence that substantially restricts expression of the second nucleotide sequence to germline cells of the arthropod. For example, the second promoter sequence may be selected from a group consisting of: zpg; nos; exu: and vasa2.

In one preferred embodiment, the second promoter sequence is referred to as “zero population growth” or “zpg”, and is provided herein as SEQ ID No: 7, as follows:

[SEQ ID No: 7]
CAGCGCTGGCGGTGGGGACAGCTCCGGCTGTGGCTGTTCTTGCGAGTCCT
CTTCCTGCGGCACATCCCTCTCGTCGACCAGTTCAGTTTGCTGAGCGTAA
GCCTGCTGCTGTTCGTCCTGCATCATCGGGACCATTTGTATGGGCCATCC
GCCACCACCACCATCACCACCGCCGTCCATTTCTAGGGGCATACCCATCA
GCATCTCCGCGGGCGCCATTGGCGGTGGTGCCAAGGTGCCATTCGTTTGT
TGCTGAAAGCAAAAGAAAGCAAATTAGTGTTGTTTCTGCTGCACACGATA
ATTTTCGTTTCTTGCCGCTAGACACAAACAACACTGCATCTGGAGGGAGA
AATTTGACGCCTAGCTGTATAACTTACCTCAAAGTTATTGTCCATCGTGG
TATAATGGACCTACCGAGCCCGGTTACACTACACAAAGCAAGATTATGCG
ACAAAATCACAGCGAAAACTAGTAATTTTCATCTATCGAAAGCGGCCGAG
CAGAGAGTTGTTTGGTATTGCAACTTGACATTCTGCTGCGGGATAAACCG
CGACGGGCTACCATGGCGCACCTGTCAGATGGCTGTCAAATTTGGCCCGG
TTTGCGATATGGAGTGGGTGAAATTATATCCCACTCGCTGATCGTGAAAA
TAGACACCTGAAAACAATAATTGTTGTGTTAATTTTACATTTTGAAGAAC
AGCACAAGTTTTGCTGACAATATTTAATTACGTTTCGTTATCAACGGCAC
GGAAAGATTATCTCGCTGATTATCCCTCTCGCTCTCTCTGTCTATCATGT
CCTGGTCGTTCTCGCGTCACCCCGGATAATCGAGAGACGCCATTTTTAAT
TTGAACTACTACACCGACAAGCATGCCGTGAGCTCTTTCAAGTTCTTCTG
TCCGACCAAAGAAACAGAGAATACCGCCCGGACAGTGCCCGGAGTGATCG
ATCCATAGAAAATCGCCCATCATGTGCCACTGAGGCGAACCGGCGTAGCT
TGTTCCGAATTTCCAAGTGCTTCCCCGTAACATCCGCATATAACAAACAG
CCCAACAACAAATACAGCATCGAG

Accordingly, preferably the second promoter sequence comprises or consists of a nucleic acid sequence substantially as set out in SEQ ID No: 7, or a variant or fragment thereof.

In another preferred embodiment, the second promoter sequence is referred to as “nanos” or “nos”, and is provided herein as SEQ ID No: 8, as follows:

[SEQ ID No: 8]
GTGAACTTCCATGGAATTACGTGCTTTTTCGGAATGGAGTTGGGCTGGTG
AAAAACACCTATCAGCACCGCACTTTTCCCCCGGCATTTCAGGTTATACG
CAGAGACAGAGACTAAATATTCACCCATTCATCACGCACTAACTTCGCAA
TAGATTGATATTCCAAAACTTTCTTCACCTTTGCCGAGTTGGATTCTGGA
TTCTGAGACTGTAAAAAGTCGTACGAGCTATCATAGGGTGTAAAACGGAA
AACAAACAAACGTTTAATGGACTGCTCCAACTGTAATCGCTTCACGCAAA
CAAACACACACGCGCTGGGAGCGTTCCTGGCGTCACCTTTGCACGATGAA
AACTGTAGCAAAACTCGCACGACCGAAGGCTCTCCGTCCCTGCTGGTGTG
TGTTTTTTTCTTTTCTGCAGCAAAATTAGAAAACATCATCATTTGACGAA
AACGTCAACTGCGCGAGCAGAGTGACCAGAAATACCGATGTATCTGTATA
GTAGAACGTCGGTTATCCGGGGGCGGATTAACCGTGCGCACAACCAGTTT
TTTGTGCAGCTTTGTAGTGTCTAGTGGTATTTTCGAAATTCATTTTTGTT
CATTAACAGTTGTTAAACCTATAGTTATTGATTAAAATAATATTCTACTA
ACGATTAACCGATGGATTCAAAGTGAATAAATTATGAAACTAGTGATTTT
TTTAAATTTTTATATGAATTTGACATTTCTTGGACCATTATCATCTTGGT
CTCGAGCTGCCCGAATAATCGACGTTCTACTGTATTCCTACCGATTTTTT
ATATGCCTACCGACACACAGGTGGGCCCCCTAAAACTACCGATTTTTAAT
TTATCCTACCGAAAATCACAGATTGTTTCATAATACAGACCAAAAAGTCA
TGTAACCATTTCCCAAATCACTTAATGTATTAAACTCCATATGGAAATCG
CTAGCAACCAGAACCAGAAGTTCAACAGAGACAACCAATTTCCGTGTATG
TACTTCATGAGATGAGATTGGACGCGCTGGTAAAATTTTATATGGGATTT
GACAGATAATGTAAGGCGTGCGATTTTTTTCATACGATGGAATCAATTCA
AGAGTCAATTGTGCAGGATTTATAGAAACAATCTCTTATTTATGTTTTGT
TATCGTTACAGTTACAGCCCTGTCCTAAGCGGCCGCGTGAAGGCCCAAAA
AAAAGGGAGTCCCCAACGCTCAGTAGCAAATGTGCTTCTCTATCATTCGT
TGGGTTAGAAAAGCCTCATGTGACTTCTATGAACAAAATCTAAACTATCT
CCTTTAAATAGAGAATGGATGTATTTTTTCGTGCCACTGAACTTTCGTTG
GGAAGATTAGATACCTCTCCCTCCCCCCCCCTCCCTTTCAACACTTCAAA
ACCTACCGAAAACTACCGATACAATTTGATGTACCTACCGAAGACCGCCA
AAATAATCTGGCCACACTGGCTAGATCTGATGTTTTGAAACATCGCCAAA
TTTTACTAAATAATGCACTTGCGCGTTGGTGAAGCTGCACTTAAACAGAT
TAGTTGAATTACGCTTTCTGAAATGTTTTTATTAAACACTTGTTTTTTTT
AATACTTCAATTTAAAGCTACTTCTTGGAATGATAATTCTACCCAAAACC
AAAACCACTTTACAAAGAGTGTGTGGTTGGTGATCGCGCCGGCTACTGCG
ACCTGTGGTCATCGCTCATCTCACGCACACATACGCACACATCTGTCATT
TGAAAAGCTGCACACAATCGTGTGTTGTGCAAAAAACCGTTCGCGCACAA
ACAGTTCGCACATGTTTGCAAGCCGTGCAGCAAAGGGCTTTTGATGGTGA
TCCGCAGTGTTTGGTCAGCTTTTTAATGTGTTTTCGCTTAATCGCTTTTG
TTTGTGTAATGTTTTGTCGGAATAATTTTTATGCGTCGTTACAAATGAAA
TGTACAATCCTGCGATGCTAGTGTAAAACATTGCTAATTCCCGGTAAGAA
CGTTCATTACGCTCGGATATCATCTTACGAAGCGTGTGTATGTGCGCTAG
TACATTGACCTTTAAAGTGATCCTTTTGTTCTAGAAAGCAAG

Accordingly, preferably the second promoter sequence comprises or consists of a nucleic acid sequence substantially as set out in SEQ ID No: 8, or a variant or fragment thereof.

In a further preferred embodiment, the second promoter sequence is referred to as “exuperantia” or “exu”, and is provided herein as SEQ ID No: 9, as follows:

[SEQ ID No: 9]
GGAAGGTGATTGCGATTCCATGTTGATGCCAATATATGATGATTTTGTTG
CATATTAATAGTTGTTGTTATGTTTTATTCAAATTTCAAAGATAATTTAC
TTTACATTACAGTTAGTGAGCATATTATCTACTACATAAACACATAGATC
AAACTGGTTTACATAAATTCAAAAAGTTTGGATTAAAATCGCAGCAATTG
GTTATGAAAAAATATGTGCATAACGTAAATATCAAGTAAATTTTTGCATT
GCATATTTATAGACTCCTGTTACAATTTCGGAAAAATGAAAAATGTTAAT
TAATCAAAGAAGAAAAAACAAAGAAATTAAATCATTAGGTAGCACAACCA
CAAGTACATATTTTTATGGCATGAATATTCCTCTACACTAACATATTTTA
TAGCAATTCTATTGATCGCCTTAGTATAGCGGAATTACCAGAACGGCACT
ATAGTTGTCTCTGTTTGGCACACGCAATCATTTTTCATCCCAGGGTTGCC
ATAGCAGTTTGGCGACGGTCACGTAGCATGCGAAGGATTTCGTTCGCACA
GGATCACTTTTATTCTAACGTTTGAAGAAGGCACATCTCAGTGCAAGCGC
TCTGGAAGCTGCTTTTACCGAACGAACTAACTTTTCAAGTAACCTCAAAA
ACTTGTCTCTAACGACACCACGTGCTATCCGCGAGTTTCATTTCCCGTGC
AAAGTTCCCCGATTTAGCTATCATTCGTGAACATTTCGTAGTGCCTCTAC
CCTCAGGTAAGACCATTCGAGGTTTACCAAGTTTTGTGCAAAGAACGTGC
ACAGTAATTTTCGTTCTGGTGAAACCTTCTCTTGTGTAGCTTGTACAAA

Accordingly, preferably the second promoter sequence comprises or consists of a nucleic acid sequence substantially as set out in SEQ ID No: 9, or a variant or fragment thereof.

In a still further preferred embodiment, the second promoter sequence is referred to as “vasa2”, and is provided herein as SEQ ID No: 10, as follows:

[SEQ ID No: 10]
ATGTAGAACGCGAGCAAATTCTTTTCCTTCCATGACAGCAGCAGCTACAG
TGGGAAGCCGAACGTCAGACGTGTTTGACATGCCGAACTGGGCGGGAAAA
TTACAGCGTGCGCTTTGTTTTCAAGCAAATCACAACTCGCTGCAAACAAA
ACCGTTGAGAAATTGATTGTTTTATAATTTGTATTGTATTTTATTTGTTA
TAATAAACTAAAAAGACATACTTTTTGCATATTTTATACATAAAAACATA
CATGCAGCATTATAAAACACATATAAACCCTCCCTGTAGAGTCCCGTATC
GAAATCTTCCATCCTAGTTGCACAGTACGACGGACGAGTAGGCCGTGTCC
GTGCAAATTCCAGCTTTTAGCAGTCTTTTGCTCGGAGCACTCGCGGCGAG
TCGGAGGTTTCTGCTGAGGTGCTTAGCGCTAAATTAGCCAATTGCTTTTG
CAAGTGAAATAACCAGCCGAATAGTACTTCAAAACTCAGGTAAGTGAACT
AGTTTTATAGAACAAATGTTTGTTTGTTAGAAGTTAGTGAAGTGTTTGTG
AAAAAAATCTCTCATTTCGGCAAAACTAACGTAACTGATTTCAAATTGAA
TTATTGTTTTGTGATGTTATATTATTTCATCCAGTTGATTAGTATTTTCT
TAGTTATGTTCAAAATACAGTTAAATTAAATTTCATTTCATTTACTCATA
AAATAATCTCTTGGCTTATTTAATTTTTCTCGAATTCGCTTGTATTGTTC
AGTAGCACGCGCCATTCGCCCTTTGTTTCATTTTGTACCTGCTCCCACTA
ACACACTGGCAGTGCGAAACAAAAGCCTTCGCACGCGTTGCTGGTATTAG
AGTGTGTGCGTGTGTGTGTTGAGCGCTCTGTCAAAATCGGCTGTTGCCGC
CGGTACCGAAATTGCCTGTTCGCACGCTGTTCGTAAACATTCCGTGGTGT
GTATCGTGTGTTGTGCATGTTGCGCGCCTCCCCCCTTTTGATAGCAGGCT
GCCGTGGCTGCCGTGGTGTGTGGCGCAGTTGAGTTTTTGGATTAATTTTC
TAAGGAAATGGCACGAGAAGAGCGGTGGCAGTGTGTTGGTTTGCTCTGTC
CCTTCCTTTCTGTGTGAAGTGTTCTTACAGCACAGCACGTATCCACCACC
GCACACAGAGCAGGCAAGGAAGTGGAAGTGAACAAGTGTGCTGCGCATGC
ATGTGTGTGGGGGGCATTTTAGCTGAGATCGTCGTTATTTGAGAAGCGGT
ATAGGGGCCAGTCGGTGTCGACGTACGGAAGCGGTTTAGTTTTAATCCAA
GCGTATCCCGTCGTGGAGTGGTTGTGTGGCTCTGTGTGCTCTCATATCAG
TTCCAGAGTGAGGTTAGTAGAATCACAGTCCTTGGCCTTTTTCGTTACAA
GATATCCAGAAGGATGGCGTTATTTCCACAGCTTACCATGGTGCTCTTGT
TTGCTCGAATCAGGGGAGAAAAACAGTTTCGTGTTTCATGAACCGCAGTT
GGCACTGGAGCGGATTCAAAAGTCTTCGATATGCAATAGATAAGAGAGTC
GTTGGGGCATAGTTGGGAAGCCTTTCCGAGATGTGGAGTTTCCGAGAGGA
GAAATGGTGCTTTCGTGCACGTTCCGGGACAGCGGGCCCCGCGAAGAGCA
TCTCGTTGTCGTTCATCCGGCAATAATTGATGCGAAAAGCGCGCGCGCCA
CTGGCTTAGCGCAGTGTACACAGTGATATTCACCTACACACACAGAGGCA
CACGCCTTCACACGCGCGCGTGCTTCAAAGGCTACTTCGGTGGCGGTGTG
TGAGGTCGCTTGCAATGGACAATGAAAATTTCGCTGGAAAATACCATCGT
CTCTTTAGGTTGCAATGGGTGCGGGTAGAGCGGTGGTCGTCGATATTGGT
GGTGTAGTGTGTGTGTGTGTGTGTGTGTGTGTGTGTGTGTGTGTGTGTGT
GTGTGTGTGTGTGTGTGTGTGTGTGTGTGTGTGTGTGTGTGTGTGTGTGT
GTGCAACGGCAATTATTTTTTGTAATATTTCGACCATCTTTCTTTCTCTC
TCTCCACGTGCTGCTGCTGTTGCTGCTGCTGCTGCATTGCATGTTCCACT
ATTCCTCTCGGTTTGTGCCTGCGGACGCCATTGCTAGTCGAAAGAGAGTC
GCCGTTAGTCGCGCTTCGAGCAACGGACACGTTTTTTGGTTGAAACCAAC
AGCTTTTTTCATCTTCGGGAGACACACAGATCTCGAATCGTACATTCCCA
TAAGGAGAATTGTCATCTTCCGGTGAATAAAGAAAGGAAAC

Accordingly, preferably the second promoter sequence comprises or consists of a nucleic acid sequence substantially as set out in SEQ ID No: 10, or a variant or fragment thereof.

Preferably, when transcribed, the first nucleotide sequence, which encodes a nucleotide sequence (i.e. the guide RNA) which hybridises to the intron-exon boundary, targets the nuclease to the intron-exon boundary of the doublesex gene. Preferably, the nuclease then cleaves the doublesex gene at the intron-exon boundary, such that the gene drive construct is integrated into the disrupted intron-exon boundary via homology-directed repair. The skilled person would understand that once the gene drive has been inserted into the genome of the arthropod, it will use the natural homology found at the site in which it is inserted in the genome.

In one embodiment, the gene drive construct is inserted into the genome via recombinase-mediated cassette exchange, a technique which would be known to those skilled in the art. Accordingly, preferably, the CRISPR-based gene drive genetic construct further comprises integrase attachment sites (preferably attB integrase attachment sites), which, respectively, flank the first nucleotide sequence encoding the nucleotide sequence that is capable of hybridising to the intron-exon boundary of the doublesex (dsx) gene, the second nucleotide sequence encoding the nuclease, the first promoter sequence and the second promoter sequence.

In one preferred embodiment, the CRISPR-based gene drive is introduced into the arthropod comprising a docking construct, wherein the docking construct comprises integrase attachment sites, preferably attP integrase attachment sites, that are flanked by 5′ and 3′ homology arms that are homologous to the genomic sequences flanking the intron-exon boundary of the arthropod, such that when the docking construct is introduced into the arthropod, it is integrated into the arthropod's genome by homology directed repair. The CRISPR-based gene drive construct is preferably inserted into the arthropod genome via recombinase-mediated cassette exchange, wherein the docking construct is exchanged for CRISPR-based gene drive construct through the action of an integrase, preferably φC31 integrase, which is introduced into the arthropod.

Preferably, the homology arms are at least 100 bp in length, at least 200 bp in length, at least 400 bp in length, at least 600 bp in length, at least 800 bp in length, at least 1000 bp in length at least 1200 bp in length, at least 1400 bp in length, at least 1600 bp in length, at least 1800 bp in length, at least 2000 bp in length. Preferably, the homology arms are up to 4000 bp in length, up to 3000 bp in length, up to 2000 bp in length. Preferably, the homology arms are between 100 and 4000 bp in length, more preferably between 150 and 3000 bp in length and most preferably between 200 and 2000 bp in length. Preferably, the homology arms are about 2000 bp in length.

In a preferred embodiment, the 5′ homology arm is provided herein as SEQ ID No: 11, as follows:

[SEQ ID No: 11]
CTTGTGTTTAGCAGGCAGGGGAGATGAGCGCAAACTGTGCAAGAAGAAGC
ATCACTGTGAAGACGGCAATGCAAAGATAGTGTGCTCAACTTCTCCGCGA
AGATTGAAGCTAAATTAAGCACGAGATTAGCATGACTGAAGTGACTTTTC
AAAGTGTCAGAATGGCTGCACTCGCAAACTAGCTGGATGCAGCGCAATTT
TGCCCCGGTGTGTGCGCGCATGCAAACGAGCAACCGCAGAGGGCAAAGGA
GAGGATGGGAAGGAGGGAGGGAGTGAAAGAGCAGGCTTAAGGTTGCCCTC
GGGCATTGAAGTCGATACAGCGGTTCTATTCCAGTGCCAGTAACGATGAC
GAAGACGATGTTGCTTCTGCTGCTGTTGCTGCTGTTGTTGTTGATGATGA
TGATGATAATAGTGCAAATATAAAATAAATCTTCCGTAAGCTTTGTGTAG
TGGTGCGTGGCTACTATAAGCCCGTCTGGAAGCAAGGAAGCTAGTCGGGC
AGGGTCATGCAAAAGGGAGACACCTTCGGAGCTCCGGAGCTCCCGCCGGC
ACTCTCGGGGGGACGTCCGTTATGCGTTGTGATTTATTATGGAATATTTA
TTATAGTGTCTTGTTTTGAAAAAATAACTTCAACGGTTCGAATTTCCTAC
ACCTCGAGATCGGGGCTGGAGTGGCAACGTGGTACGGAACGGTACAGCGG
TTTGAGCCGTTCGGTCTTGGGACTCACGGATCGCAGAATGTTATTGTGCG
CGCACTGATGGGAAAGTCATTTTTCACCGAGTGGTCAGGGCGCGTAGTCC
AGTTCGTTTCTGGCTGCTGTTGCTGATGCTACGATCCTCAGGAATGATTG
GAAACGCCTGGAGATGGTGGGAAAAAATCAAACACAAAAACGATCCTAAT
GAACATCGTGTGTTCTCATTCGCTGCCACGATTGACACCTTCGATAAGAC
GCACATAATGAGCTAAAGGAGAGGGGACAGGGTCTTGTCTTTGCCACGAG
CGATAAGATTGCAATCACTCGTGAGCGTGTGCTGCTGGGCTGAAGAAGAA
ACGCTTTCCACAGCAGTAGGTGGGAAGTGGGATTGTGGAACGTGGCATTG
AAAAGAACCTATTTTCTAAAGCCCGAGAGCCCGTTCTCGAACTGGAAAAC
CAGATGCAGAAGTTTTTTATTGTCCCCCGCCAGGAAAACAAATGTATTTA
ATGCTTTCTTTGCCTTTTCCGCCCCGTTTCAGACGACGAGCTAGTGAAGC
GAGCCCAATGGCTGTTGGAGAAACTCGGCTACCCGTGGGAGATGATGCCC
CTGATGTACGTCATACTAAAGAGCGCCGATGGCGATGTACAAAAAGCACA
CCAGCGGATCGACGAAGGTAAGCTGGCGATGATGGTGTCGTTCGACATCA
CTTTCATCACCGTGTCAGACATCTACTGTGCCTAGCACCGGGTCCAGTGG
TCACAGGGTGTAGCAAAAACGTGTTCTTTTTTGCGAGAGACTCTACCTCA
TGATGCAGCTGTTAAGGAAAGGTTTCAGATGAAGGCAATTTTTCCTAGGA
TAAGATGATCTTAAGTTACCTGCGTATTAGTGTTTAACATTGTCGTCTCA
ACTCCCAAGAATGTTTTAATCGTCTAGGGCTAGTTTATTTATACTGTTCT
CATTGAAATGTCGTTCAATCCAACATGTTAAGTTAGCTAGCTCAGACACG
AGAAGTTAGGAGTATCTGCATCTTGAAGGTAGCGGCATATGGTGTTATGC
CACGTTCACTGACTTCAAAATTCGATACAAAAAAAAAACCAAAACATCAA
AAACCAAATTGTGAATTCCGTCAGCCAGCAGCAGTGACCTTCAAAGCCTT
ACCTTTCCATTCATTTATGTTTAACACAGGTCAAG

Accordingly, preferably the 5′ homology arm comprises or consists of a nucleic acid sequence substantially as set out in SEQ ID No: 11, or a variant or fragment thereof.

In a preferred embodiment, the 3′ homology arm is provided herein as SEQ ID No: 12, as follows:

[SEQ ID No: 12]
CGGTGGTCAACGAATACTCACGATTGCATAATCTGAACATGTTTGATGGC
GTGGAGTTGCGCAATACCACCCGTCAGAGTGGATGATAAACTTTCCGCAC
CACTGTAACTGTCCGTATCTTTGTATGTGGGTGTGTGTATGTGTGTTTGG
TGAAACGAATTCAATAGTTCTGTGCTATTTTAAATCAAGCCGCGTGCGCA
ACTGATGCCGATAAGTTCAAACTAGTGTTTAAGGAGTGGAGCGAGAGAGC
CGCACCACGGTACAGAAGGGCAGCAGAATGGGTCGGCAGCCTAGCTGCAC
TGGTGCGGTGCGTCCGGCGTCTCGGGGGGAGGGCGAGGAAATTCTAGTGT
TAAATCGGAGCAGCAAAAACAAAACAGTGGTCGTCCCGTTCAAGAAACGG
CCTGTACACACACACAGAAAACACTGCAGCATGTTTGTACATAGTAGATC
CTAGAGCAGGTGGTCGTTGCTCCTCGAACGCTCTGGACGCACGGCTTCGC
GCGTATTTGCGTAGCGTTCCGCCGATCGTGGGTATTCGTACTGCCACAAG
CCCGCTTTCTCCCATGCAATCTCTGCAACCAAACCAACAAACAACAACAA
AAAACCAATCGACAAAATGAATCACACCCCTTTTGTATCATCTGTATATT
CTTGTTCTTTGCGTTCTTTTCTATGTGGCCCACGCCCCGGCGGGTACGTA
ATTGCGTCGAAAACCCCGAAAACCCCGGCACATACAGTGTACATACGGTT
TGAGGACAACTTTGACCTGCAGCCCTTCTGGGGTTGCCACGTGTAGCTAT
ACTTGTGAGATCGGGCGCCGACGGTGTAAAGCGCGAATGGCCGCCACACA
GTGTGTCCACTCCAACACTACCCCTCTGGAACTACCCCGTCCAGGGATGC
ACCGGCTCGGCTCATGCCCCTGCAAAACAGTCCGGGCTCCACTGTAGTAG
CTCCGGCGTTGCTCTGAGAGAAGGATGCCCTTCGAAGTGTCGAAAGCGTG
CATTGGGCGTTCAAGTGTGTGTGTGTGTGTTAGGTTTAGCGAGAAACAGC
AGCAGTTGCGTGTGCTGAAAAGCGAAGGAGTAATAGAGTGCATAATGAAA
ATGAAAATGAAAATGAAGCAAAAGTAGAAGGCGGAGGAGAGCAACCTGTG
TTCCACTAGTAGCGAATAGTTTAGTCTAGTTTCGTCACCAATCAACCTTC
CAACCATCGTTCAACCAATACCTGAGTCAACATCGTCATCGTTATCGTGC
CACAACTTTATTAAAAATGAACCTTGTCCGCGCCACCGTAGGGTGATCTA
AGGCGACCTTTCTTACGGGCGCGACCCACATGCCATCGTCACCTTCTCCA
ATCAAAACCAACAGCCTGTACCGATGGTGTGCAATTGTGCGTGCGTGTGT
GTTATTAGCAAAAAAAGAGAAAGAGTCGACGAGAGAGAGATAGATCGAGA
TCGAGAGTACAAAAGAGCAGTAGAAATGTTCGTTGTTTGTTTTTCGTAAC
ACAGTTGTTTAGCCAAAATGGGAATTTCCAATAATCCCGGGGGCGGGGAA
ATGCGGGAATACTGCGTACACACATACATCAATCAAAAAGAAAAATCCTT
GCGCTACATCACTACCGTTTGCGCGGTGCTGATCTAGAGCAGACCACTTT
CCACTCCACTCTACAATCAATCAATCTGTGCAGAAGGTATGGTAAGACGG
CCTTTGAGCGAGTCACGGTCGCCACCATAACGCCGTCCGACGAGGGCTGA
ATGCGAACTTTGCTAATCGATTTTCCGCTTTCTTTTTATCCCACCTCCTT
TTCTCTCCCTCTCTCTCTTTTGCACTGCCCCTTGTAACCCCCAAAAAGGT
AAACGACACATTAAGACCTACGAAGCGTTGGTGAAGTCATCGCTCGATCC
GAACAGCGACCGGCTGACGGAGGACGACGACGAGGACGAGAACATCTCGG
TGACCCGCACC

Accordingly, preferably the 3′ homology arm comprises or consists of a nucleic acid sequence substantially as set out in SEQ ID No: 12, or a variant or fragment thereof.

In another embodiment, the CRISPR-based gene drive construct may instead be inserted into the genome by homology-directed repair, i.e. without the use of a docking construct, as described above. Accordingly, preferably, the CRISPR-based gene drive genetic construct further comprises third and fourth nucleotide sequences which, respectively, flank the first nucleotide sequence encoding the nucleotide sequence that is capable of hybridising to the intron-exon boundary of the doublesex (dsx) gene, the second nucleotide sequence encoding the nuclease, the first promoter sequence and the second promoter sequence, wherein the third and fourth nucleotides are homologous to the genomic sequences flanking the intron-exon boundary, such that the gene drive construct is integrated into the genome via homology-directed repair.

Preferably, the third and fourth nucleotide sequences are at least 100 bp in length, at least 200 bp in length, at least 400 bp in length, at least 600 bp in length, at least 800 bp in length, at least 1000 bp in length at least 1200 bp in length at least 1400 bp in length, at least 1600 bp in length, at least 1800 bp in length, at least 2000 bp in length. Preferably, the third and fourth nucleotide sequences are up to 4000 bp in length, up to 3000 bp in length, up to 2000 bp in length. Preferably, the third and fourth nucleotide sequences are between 100 and 4000 bp in length, more preferably between 150 and 3000 bp in length and most preferably between 200 and 2000 bp in length. Preferably, the third and fourth nucleotide sequences are about 2000 bp in length.

Accordingly, preferably the third nucleotide sequence comprises or consists of a nucleic acid sequence substantially as set out in SEQ ID No: 11, or a variant or fragment thereof.

Accordingly, preferably the fourth nucleotide sequence comprises or consists of a nucleic acid sequence substantially as set out in SEQ ID No: 12, or a variant or fragment thereof.

Preferably, the CRISPR-based gene drive construct targets the intron-4-exon 5 boundary of the doublesex gene.

In a preferred embodiment, the gene drive construct is provided herein as SEQ ID No: 13, as follows:

[SEQ ID No: 13]
TGCGGGTGCCAGGGCGTGCCCTTGGGCTCCCCGGGCGCGTACTCCACCTCACCCATGCGATCGCTCCGGAAAGATACATTGATGAGTT
TGGACAAACCACAACTAGAATGCAGTGAAAAAAATGCTTTATTTGTGAAATTTGTGATGCTATTGCTTTATTTGTAACCATTATAAGC
TGCAATAAACAAGTTAACAACAACAATTGCATTCATTTTATGTTTCAGGTTCAGGGGGAGGTGTGGGAGGTTTTTTAAAGCAAGTAAA
ACCTCTACAAATGTGGTATGGCTGATTATGATCTAGAGTCGCGGCCGCTACAGGAACAGGTGGTGGCGGCCCTCGGTGCGCTCGTACT
GCTCCACGATGGTGTAGTCCTCGTTGTGGGAGGTGATGTCCAGCTTGGAGTCCACGTAGTAGTAGCCGGGCAGCTGCACGGGCTTCTT
GGCCATGTAGATGGACTTGAACTCCACCAGGTAGTGGCCGCCGTCCTTCAGCTTCAGGGCCTTGTGGATCTCGCCCTTCAGCACGCCG
TCGCGGGGGTACAGGCGCTCGGTGGAGGCCTCCCAGCCCATGGTCTTCTTCTGCATTACGGGGCCGTCGGAGGGGAAGTTCACGCCGA
TGAACTTCACCTTGTAGATGAAGCAGCCGTCCTGCAGGGAGGAGTCTTGGGTCACGGTCACCACGCCGCCGTCCTCGAAGTTCATCAC
GCGCTCCCACTTGAAGCCCTCGGGGAAGGACAGCTTCTTGTAGTCGGGGATGTCGGCGGGGTGCTTCACGTACACCTTGGAGCCGTAC
TGGAACTGGGGGGACAGGATGTCCCAGGCGAAGGGCAGGGGGCCGCCCTTGGTCACCTTCAGCTTCACGGTGTTGTGGCCCTCGTAGG
GGCGGCCCTCGCCCTCGCCCTCGATCTCGAACTCGTGGCCGTTCACGGTGCCCTCCATGCGCACCTTGAAGCGCATGAACTCCTTGAT
GACGTTCTTGGAGGAGCGCACCATGGTGGCGACCTGTGGGTCCCGGGCCCGCGGTACCGTCGACTCTAGCGGTACCCCGATTGTTTAG
CTTGTTCAGCTGCGCTTGTTTATTTGCTTAGCTTTCGCTTAGCGACGTGTTCACTTTGCTTGTTTGAATTGAATTGTCGCTCCGTAGA
CGAAGCGCCTCTATTTATACTCCGGCGGTCGAGGGTTCGAAATCGATAAGCTTGGATCCTAATTGAATTAGCTCTAATTGAATTAGTC
TCTAATTGAATTAGATCCCCGGGCGAGCTCGAATTAACCATTGTGGACCGGTCAGCGCTGGCGGTGGGGACAGCTCCGGCTGTGGCTG
TTCTTGAGAGTCATCTTCCTGCGGCACATCCCTCTCGTCGACCAGTTCAGTTTGCTGAGCGTAAGCCTGCTGCTGTTCGTCCTGCATC
ATCGGGACCATTTGTACGGGCCATCCGCCACCACCACCATCACCACCGCCGTCCATTTCTAGGGGCATACCCATCAGCATCTCCGCGG
GCGCCATTGGCGGTGGTGCCAAGGTGCCATTCGTTTGTTGCTGAAAGCAAAAGAAAGCAAATTAGTGTTGTTTCTGCTGCACACGATA
GTTTTCGTTTCTTGCCGCTAGACACAAACAACACTGCATCTGGAGGGAGAAATTTGACGCCTAGCTGTATAACTTACCTCAAAGTTAT
TGTCCATCGTGGTATAATGGACCTACCGAGCCCGGTTACACTACACAAAGCAAGATTATGCGACAAAATCACAGCGAAAACTAGTAAT
TTTCATCTATCGAAAGCGGCCGAGCAGAGAGTTGTTTGGTATTGCAACTTGACATTCTGCTGTGGGATAAACCGCGACGGGCTACCAT
GGCGCACCTGTCAGATGGCTGTCAAATTTGGCCCGGTTTGCGATATGGAGTGGGTGAAATTATATCCCACTCGCTGATCGTGAAAATA
GACACCTGAAAACAATAATTGTTGTGTTAATTTTACATTTTGAAGAACAGCACAAGTTTTGCTGACAATATTTAATTACGTTTCGTTA
TCAACGGCACGGAAAGATTATCTCGCTGATTATCCCTCTCGCTCTCTCTGTCTATCATGTCCTGGTCGTTCTCGCGTCACCCCGGATA
ATCGAGAGACGCCATTTTTAATTTGAACTACTACACCGACAAGCATGCCGTGAGCTCTTTCAAGTTCTTCTGTCCGACCAAAGAAACA
GAGAATACCGCCCGGACAGTGCCCGGAGTGATCGATCCATAGAAAATCGCCCATCATGTGCCACTGAAGCGAACCGGCGTAGCTTGTT
CCGAATTTCCAAGTGCTTCCCCGTAACATCCGCATATAACAAGCAGCCCAACAACAAATACAGCATCGAGCTCGAGATGGACTATAAG
GACCACGACGGAGACTACAAGGATCATGATATTGATTACAAAGACGATGACGATAAGATGGCCCCAAAGAAGAAGCGGAAGGTCGGTA
TCCACGGAGTCCCAGCAGCCGACAAGAAGTACAGCATCGGCCTGGACATCGGCACCAACTCTGTGGGCTGGGCCGTGATCACCGACGA
GTACAAGGTGCCCAGCAAGAAATTCAAGGTGCTGGGCAACACCGACCGGCACAGCATCAAGAAGAACCTGATCGGAGCCCTGCTGTTC
GACAGCGGCGAAACAGCCGAGGCCACCCGGCTGAAGAGAACCGCCAGAAGAAGATACACCAGACGGAAGAACCGGATCTGCTATCTGC
AAGAGATCTTCAGCAACGAGATGGCCAAGGTGGACGACAGCTTCTTCCACAGACTGGAAGAGTCCTTCCTGGTGGAAGAGGATAAGAA
GCACGAGCGGCACCCCATCTTCGGCAACATCGTGGACGAGGTGGCCTACCACGAGAAGTACCCCACCATCTACCACCTGAGAAAGAAA
CTGGTGGACAGCACCGACAAGGCCGACCTGCGGCTGATCTATCTGGCCCTGGCCCACATGATCAAGTTCCGGGGCCACTTCCTGATCG
AGGGCGACCTGAACCCCGACAACAGCGACGTGGACAAGCTGTTCATCCAGCTGGTGCAGACCTACAACCAGCTGTTCGAGGAAAACCC
CATCAACGCCAGCGGCGTGGACGCCAAGGCCATCCTGTCTGCCAGACTGAGCAAGAGCAGACGGCTGGAAAATCTGATCGCCCAGCTG
CCCGGCGAGAAGAAGAATGGCCTGTTCGGAAACCTGATTGCCCTGAGCCTGGGCCTGACCCCCAACTTCAAGAGCAACTTCGACCTGG
CCGAGGATGCCAAACTGCAGCTGAGCAAGGACACCTACGACGACGACCTGGACAACCTGCTGGCCCAGATCGGCGACCAGTACGCCGA
CCTGTTTCTGGCCGCCAAGAACCTGTCCGACGCCATCCTGCTGAGCGACATCCTGAGAGTGAACACCGAGATCACCAAGGCCCCCCTG
AGCGCCTCTATGATCAAGAGATACGACGAGCACCACCAGGACCTGACCCTGCTGAAAGCTCTCGTGCGGCAGCAGCTGCCTGAGAAGT
ACAAAGAGATTTTCTTCGACCAGAGCAAGAACGGCTACGCCGGCTACATTGACGGCGGAGCCAGCCAGGAAGAGTTCTACAAGTTCAT
CAAGCCCATCCTGGAAAAGATGGACGGCACCGAGGAACTGCTCGTGAAGCTGAACAGAGAGGACCTGCTGCGGAAGCAGCGGACCTTC
GACAACGGCAGCATCCCCCACCAGATCCACCTGGGAGAGCTGCACGCCATTCTGCGGCGGCAGGAAGATTTTTACCCATTCCTGAAGG
ACAACCGGGAAAAGATCGAGAAGATCCTGACCTTCCGCATCCCCTACTACGTGGGCCCTCTGGCCAGGGGAAACAGCAGATTCGCCTG
GATGACCAGAAAGAGCGAGGAAACCATCACCCCCTGGAACTTCGAGGAAGTGGTGGACAAGGGCGCTTCCGCCCAGAGCTTCATCGAG
CGGATGACCAACTTCGATAAGAACCTGCCCAACGAGAAGGTGCTGCCCAAGCACAGCCTGCTGTACGAGTACTTCACCGTGTATAACG
AGCTGACCAAAGTGAAATACGTGACCGAGGGAATGAGAAAGCCCGCCTTCCTGAGCGGCGAGCAGAAAAAGGCCATCGTGGACCTGCT
GTTCAAGACCAACCGGAAAGTGACCGTGAAGCAGCTGAAAGAGGACTACTTCAAGAAAATCGAGTGCTTCGACTCCGTGGAAATCTCC
GGCGTGGAAGATCGGTTCAACGCCTCCCTGGGCACATACCACGATCTGCTGAAAATTATCAAGGACAAGGACTTCCTGGACAATGAGG
AAAACGAGGACATTCTGGAAGATATCGTGCTGACCCTGACACTGTTTGAGGACAGAGAGATGATCGAGGAACGGCTGAAAACCTATGC
CCACCTGTTCGACGACAAAGTGATGAAGCAGCTGAAGCGGCGGAGATACACCGGCTGGGGCAGGCTGAGCCGGAAGCTGATCAACGGC
ATCCGGGACAAGCAGTCCGGCAAGACAATCCTGGATTTCCTGAAGTCCGACGGCTTCGCCAACAGAAACTTCATGCAGCTGATCCACG
ACGACAGCCTGACCTTTAAAGAGGACATCCAGAAAGCCCAGGTGTCCGGCCAGGGCGATAGCCTGCACGAGCACATTGCCAATCTGGC
CGGCAGCCCCGCCATTAAGAAGGGCATCCTGCAGACAGTGAAGGTGGTGGACGAGCTCGTGAAAGTGATGGGCCGGCACAAGCCCGAG
AACATCGTGATCGAAATGGCCAGAGAGAACCAGACCACCCAGAAGGGACAGAAGAACAGCCGCGAGAGAATGAAGCGGATCGAAGAGG
GCATCAAAGAGCTGGGCAGCCAGATCCTGAAAGAACACCCCGTGGAAAACACCCAGCTGCAGAACGAGAAGCTGTACCTGTACTACCT
GCAGAATGGGCGGGATATGTACGTGGACCAGGAACTGGACATCAACCGGCTGTCCGACTACGATGTGGACCATATCGTGCCTCAGAGC
TTTCTGAAGGACGACTCCATCGACAACAAGGTGCTGACCAGAAGCGACAAGAACCGGGGCAAGAGCGACAACGTGCCCTCCGAAGAGG
TCGTGAAGAAGATGAAGAACTACTGGCGGCAGCTGCTGAACGCCAAGCTGATTACCCAGAGAAAGTTCGACAATCTGACCAAGGCCGA
GAGAGGCGGCCTGAGCGAACTGGATAAGGCCGGCTTCATCAAGAGACAGCTGGTGGAAACCCGGCAGATCACAAAGCACGTGGCACAG
ATCCTGGACTCCCGGATGAACACTAAGTACGACGAGAATGACAAGCTGATCCGGGAAGTGAAAGTGATCACCCTGAAGTCCAAGCTGG
TGTCCGATTTCCGGAAGGATTTCCAGTTTTACAAAGTGCGCGAGATCAACAACTACCACCACGCCCACGACGCCTACCTGAACGCCGT
CGTGGGAACCGCCCTGATCAAAAAGTACCCTAAGCTGGAAAGCGAGTTCGTGTACGGCGACTACAAGGTGTACGACGTGCGGAAGATG
ATCGCCAAGAGCGAGCAGGAAATCGGCAAGGCTACCGCCAAGTACTTCTTCTACAGCAACATCATGAACTTTTTCAAGACCGAGATTA
CCCTGGCCAACGGCGAGATCCGGAAGCGGCCTCTGATCGAGACAAACGGCGAAACCGGGGAGATCGTGTGGGATAAGGGCCGGGATTT
TGCCACCGTGCGGAAAGTGCTGAGCATGCCCCAAGTGAATATCGTGAAAAAGACCGAGGTGCAGACAGGCGGCTTCAGCAAAGAGTCT
ATCCTGCCCAAGAGGAACAGCGATAAGCTGATCGCCAGAAAGAAGGACTGGGACCCTAAGAAGTACGGCGGCTTCGACAGCCCCACCG
TGGCCTATTCTGTGCTGGTGGTGGCCAAAGTGGAAAAGGGCAAGTCCAAGAAACTGAAGAGTGTGAAAGAGCTGCTGGGGATCACCAT
CATGGAAAGAAGCAGCTTCGAGAAGAATCCCATCGACTTTCTGGAAGCCAAGGGCTACAAAGAAGTGAAAAAGGACCTGATCATCAAG
CTGCCTAAGTACTCCCTGTTCGAGCTGGAAAACGGCCGGAAGAGAATGCTGGCCTCTGCCGGCGAACTGCAGAAGGGAAACGAACTGG
CCCTGCCCTCCAAATATGTGAACTTCCTGTACCTGGCCAGCCACTATGAGAAGCTGAAGGGCTCCCCCGAGGATAATGAGCAGAAACA
GCTGTTTGTGGAACAGCACAAGCACTACCTGGACGAGATCATCGAGCAGATCAGCGAGTTCTCCAAGAGAGTGATCCTGGCCGACGCT
AATCTGGACAAAGTGCTGTCCGCCTACAACAAGCACCGGGATAAGCCCATCAGAGAGCAGGCCGAGAATATCATCCACCTGTTTACCC
TGACCAATCTGGGAGCCCCTGCCGCCTTCAAGTACTTTGACACCACCATCGACCGGAAGAGGTACACCAGCACCAAAGAGGTGCTGGA
CGCCACCCTGATCCACCAGAGCATCACCGGCCTGTACGAGACACGGATCGACCTGTCTCAGCTGGGAGGCGACAAAAGGCCGGCGGCC
ACGAAAAAGGCCGGCCAGGCAAAAAAGAAAAAGTAATTAATTAAGAGGACGGCGAGAAGTAATCATATGTCCGCATTTTGCGCAAACC
AGGCGCTTAGACAATTTGCGCGTAAGCACATTCGAAATGTGAAAAGCTGAAAGCAGTGGTTTCGCCAGCCCGAGTTCAGCGAAACGGA
TTCCTTCCAAGTGTTTGCATTCCTGGCGGAGTGTTCCTCCCAAAATGCACTCACCCTGCGTGCAGTGCCAAATCGTGAGTTTCCTAAT
TTTTTCATATTGTTTATTACCTACCAACTAAAGTTGTTGTTATATATTGCGTTTTACGTACGACAAATAAGTTCGTATTCAGAAATAT
TTGCGATAAGAGAGAACTCATTTGCGATGAATCTCATTGTATTTAGCTAAGTGCCTTGATAAGTAAGCGGAACAGCAGGAATATGACA
CTCCTTGGGAAATACATGTAAGCGTCTGTAATTAGATATATATACACGCAACCAAATGGTCCATGGTTGATTTAAGCACTGCCTGTTG
TCGAACATTGCTATAAGCAAAATAAAGAAGCATTCATTAATCTAAAATTTCTTCAAAGTGACTTCAATGATGATCTCTAGGCTATAGT
GAAAGCTGAAAGCTTATTTGACAATGCAAGGGAAAGTGACGCACGTGCGTCGTATGGGACCGCGCGCATCTATTCTCTCAGCTAATTC
CCCTAATCATTAGTAATTGACGGCACGATTTCTGCTTCTTACTTCCTTTTACTTTGGAGCTTTTCATCAATAAAACCAGTACCATGGC
CGTACGCTCAACGGAAAAGCATTCAAAAAAACCCGCGTTCCTCGTGTGATTTGTGGGTGAGTGGCGCCATCTATTAGAGAATAGCTGT
ACTACATCTCGTGGACGAAGGGGTCAGAGAAGTTGAAAGAGAGCTTGATCGACTGCTATCCAAGCTAGGCGAGGAAGGGAGATCGCTA
GAGCAAAAGAAAAAAAATAAGCAAATATCTTTTTTTATAACAAATCGACGTTAGCGAAATATGTTTGAATCGATTTAACGGTTAGAAT
TCCCTTTGGTTCGTTCATTATGCGAGGCGCGCCTTTGTATGCGTGCGCTTGAAGGGTTGATCGGAACCTTACAACAGTTGTAGCTATA
CGGCTGCGTGTGGCTTCTAACGTTATCCATCGCTAGAAGTGAAACGAATGTGCGTAGGTATATATATGAAATGGAGTTGCTCTCTGCT
GTTTAACACAGGTCAAGCGGGTTTTAGAGCTAGAAATAGCAAGTTAAAATAAGGCTAGTCCGTTATCAACTTGAAAAAGTGGCACCGA
GTCGGTGCTTTTTTTTACGCGTGGGTCCCATGGGTGAGGTGGAGTACGCGCCCGGGGAGCCCAAGGGCACGCCCTGGCACCCGCA

Accordingly, preferably the gene drive construct comprises or consists of a nucleic acid sequence substantially as set out in SEQ ID NO: 13, or a fragment or variant thereof.

The gene drive construct may for example be a plasmid, cosmid or phage and/or be a viral vector. Such recombinant vectors are highly useful in the delivery systems of the invention for transforming cells. The nucleic acid sequence may preferably be a DNA sequence. The gene drive construct may further comprise a variety of other functional elements including a suitable regulatory sequence for controlling expression of the genetic gene drive construct upon introduction of the construct in a host cell. The construct may further comprise a regulator or enhancer to control expression of the elements of the constructs required. Tissue specific enhancer elements, for example promoter sequences, may be used to further regulate expression of the construct in germ cells of an arthropod.

Thus, it will be appreciated that the inventors have developed in the human malaria vector Anopheles gambiae a CRISPR-based gene drive that selectively impairs mosquito embryos in producing the female splice transcript of the sex determining gene doublesex. Advantageously, the female's reproductive capacity is suppressed only in female insects homozygous for the disrupted allele, which may show an intersex phenotype characterised by the presence of male internal and external reproductive organs and complete sterility. Heterozygous females may remain fertile and may be capable of producing transformed progeny. In addition, development and fertility may be unaffected in those males heterozygous or homozygous for the disrupted allele. This has the effect of enabling the gene drive to reach a high proportion of the insect population.

Furthermore, by targeting the highly conserved and constrained doublesex intron-4-exon 5 boundary, the drive does not induce resistance, even when a variety of non-functional nuclease resistant variants are generated in each generation at the target site. Nevertheless, the inventors have carefully considered various innovative approaches that may be used to mitigate any against possible resistance to gene drive, and have successfully demonstrated that one option is to target multiple sites at the same time, because, for resistance to get selected against the gene drive, resistant mutations would have to be simultaneously present at all target sites, and co-operatively restore the targeted gene's original function. It will be appreciated that homing can also serve to remove resistant mutations generated if at least one of the multiple targeted sites is still cleavable.

The inventors have analysed the sequence of Exon 5 of doublesex and found that it surprisingly contains at least four invariant (i.e. highly conserved and constrained) target sites that are amenable to multiplexing (i.e. targeting more than one site simultaneously), which are shown in FIG. 12 as T1, T2, T3 and T4. Accordingly, the inventors generated a novel multiplexed gene drive system targeting not only the original target site at doublesex (i.e. the intron-exon boundary of the female specific splice form of the dsx gene, referred to in FIG. 12 as T1), but also one or more additional target sites selected from T2, T3 and T4, which are present at or towards the 3′ end of the exon 5 coding sequence. The inheritance bias of the gene drive, and fertility of gene drive carriers was assessed through phenotype assays, and the inventors found that the novel multiplexed gene drive successfully biased its inheritance to the next generation with transmission rates comparable to the single-guide gene drive, but with the added advantage that any resistance mutations to gene drive are significantly mitigated.

Accordingly, in an embodiment, the gene drive genetic construct of the invention may be capable of targeting (i) a first target site which comprises an intron-exon boundary of the female specific splice form of the doublesex (dsx) gene, and (ii) a second target site disposed in exon 5 of the female specific splice form of the doublesex (dsx) gene.

The genomic nucleotide sequence of exon 5 of the doublesex (dsx) gene is provided herein as SEQ ID No: 35, as follows:

[SEQ ID No: 35]
GTCAAGCGGTGGTCAACGAATACTCACGATTGCATAATCTGAACATGTTT
GATGGCGTGGAGTTGCGCAATACCACCCGTCAGAGTGGATGATAAACTTT
CCGCACCACTGTAACTGTCCGTATCTTTGTATGTGGGTGTGTGTATGTGT
GTTTGGTGAAACGAATTCAATAGTTCTGTGCTATTTTAAATCAAGCCGCG
TGCGCAACTGATGCCGATAAGTTCAAACTAGTGTTTAAGGAGTGGAGCGA
GAGAGCCGCACCACGGTACAGAAGGGCAGCAGAATGGGTCGGCAGCCTAG
CTGCACTGGTGCGGTGCGTCCGGCGTCTCGGGGGGAGGGCGAGGAAATTC
TAGTGTTAAATCGGAGCAGCAAAAACAAAACAGTGGTCGTCCCGTTCAAG
AAACGGCCTGTACACACACACAGAAAACACTGCAGCATGTTTGTACATAG
TAGATCCTAGAGCAGGTGGTCGTTGCTCCTCGAACGCTCTGGACGCACGG
CTTCGCGCGTATTTGCGTAGCGTTCCGCCGATCGTGGGTATTCGTACTGC
CACAAGCCCGCTTTCTCCCATGCAATCTCTGCAACCAAACCAACAAACAA
CAACAAAAAACCAATCGACAAAATGAATCACACCCCTTTTGTATCATCTG
TATATTCTTGTTCTTTGCGTTCTTTTCTATGTGGCCCACGCCCCGGCGGG
TACGTAATTGCGTCGAAAACCCCGAAAACCCCGGCACATACAGTGTACAT
ACGGTTTGAGGACAACTTTGACCTGCAGCCCTTCTGGGGTTGCCACGTGT
AGCTATACTTGTGAGATCGGGCGCCGACGGTGTAAAGCGCGAATGGCCGC
CACACAGTGTGTCCACTCCAACACTACCCCTCTGGAACTACCCCGTCCAG
GGATGCACCGGCTCGGCTCATGCCCCTGCAAAACAGTCCGGGCTCCACTG
TAGTAGCTCCGGCGTTGCTCTGAGAGAAGGATGCCCTTCGAAGTGTCGAA
AGCGTGCATTGGGCGTTCAAGTGTGTGTGTGTGTGTTAGGTTTAGCGAGA
AACAGCAGCAGTTGCGTGTGCTGAAAAGCGAAGGAGTAATAGAGTGCATA
ATGAAAATGAAAATGAAAATGAAGCAAAAGTAGAAGGCGGAGGAGAGCAA
CCTGTGTTCCACTAGTAGCGAATAGTTTAGTCTAGTTTCGTCACCAATCA
ACCTTCCAACCATCGTTCAACCAATACCTGAGTCAACATCGTCATCGTTA
TCGTGCCACAACTTTATTAAAAATGAACCTTGTCCGCGCCACCGTAGGGT
GATCTAAGGCGACCTTTCTTACGGGCGCGACCCACATGCCATCGTCACCT
TCTCCAATCAAAACCAACAGCCTGTACCGATGGTGTGCAATTGTGCGTGC
GTGTGTGTTATTAGCAAAAAAAGAGAAAGAGTCGACGAGAGAGAGATAGA
TCGAGATCGAGAGTACAAAAGAGCAGTAGAAATGTTCGTTGTTTGTTTTT
CGTAACACAGTTGTTTAGCCAAAATGGGAATTTCCAATAATCCCGGGGGC
GGGGAAATGCGGGAATACTGCGTACACACATACATCAATCAAAAAGAAAA
ATCCTTGCGCTACATCACTACCGTTTGCGCGGTGCTGATCTAGAGCAGAC
CACTTTCCACTCCACTCTACAATCAATCAATCTGTGCAGAAGGTATGGTA
AGACGGCCTTTG

In one embodiment, therefore, the second target site comprises or consists of a nucleic acid sequence, which is disposed in the sequence substantially as set out in SEQ ID No: 35, or a variant or fragment thereof. In some embodiments, the genetic construct targets a second target site comprising or consisting of the nucleotide sequence substantially as set out in SEQ ID NO: 35, or a fragment or variant thereof. The second target site may include up to 1, 2, 3, 4, 5, 10 or 15 nucleotides 5′ and/or 3′ of SEQ ID No:35.

As shown in FIG. 12, the second target site may be the sequence shown as T2, which is provided herein as SEQ ID No: 36, as follows:

[SEQ ID No: 36]
TCTGAACATGTTTGATGGCGTGG

In one embodiment, therefore, the second target site comprises or consists of a nucleic acid sequence substantially as set out in SEQ ID No: 36, or a variant or fragment thereof. In some embodiments, the genetic construct targets a second target site comprising or consisting of the nucleotide sequence substantially as set out in SEQ ID NO: 36, or a fragment or variant thereof. The second target site may include up to 1, 2, 3, 4, 5, 10 or 15 nucleotides 5′ and/or 3′ of SEQ ID No:36. As is shown in FIG. 12, T2 is wholly contained within exon 5.

The second target site may be the sequence shown as T3, which is provided herein as SEQ ID No: 37, as follows:

[SEQ ID No: 37]
GCAATACCACCCGTCAGAGTGG

In one embodiment, therefore, the second target site comprises or consists of a nucleic acid sequence substantially as set out in SEQ ID No: 37, or a variant or fragment thereof. In some embodiments, the genetic construct targets a second target site comprising or consisting of the nucleotide sequence substantially as set out in SEQ ID NO: 37, or a fragment or variant thereof. The second target site may include up to 1, 2, 3, 4, 5, 10 or 15 nucleotides 5′ and/or 3′ of SEQ ID No:37. As is shown in FIG. 12, T3 is wholly contained within exon 5.

The second target site may be the sequence shown as T4, which is provided herein as SEQ ID No: 38, as follows:

[SEQ ID No: 38]
GTTTATCATCCACTCTGACGG

In one embodiment, therefore, the second target site comprises or consists of a nucleic acid sequence substantially as set out in SEQ ID No: 38, or a variant or fragment thereof. In some embodiments, the genetic construct targets a second target site comprising or consisting of the nucleotide sequence substantially as set out in SEQ ID NO: 38, or a fragment or variant thereof. The second target site may include up to 1, 2, 3, 4, 5, 10 or 15 nucleotides 5′ and/or 3′ of SEQ ID No:38. As is shown in FIG. 12, T4 is partially in the 3′ end of exon 5 and extends into the untranslated region of exon 5.

The gene drive construct of the invention may target one or more of a second target site selected from a group consisting of T2, T3 and T4. Most preferably, the gene drive genetic construct of the invention targets T1 and one or more of T2, T3 and T4. For example, the construct may target T1 and T2, or T1 and T3, or T1 and T4, or T1, T2 and T3, T1, T2 and T4, or T1 and T3 and T4, or any combination thereof.

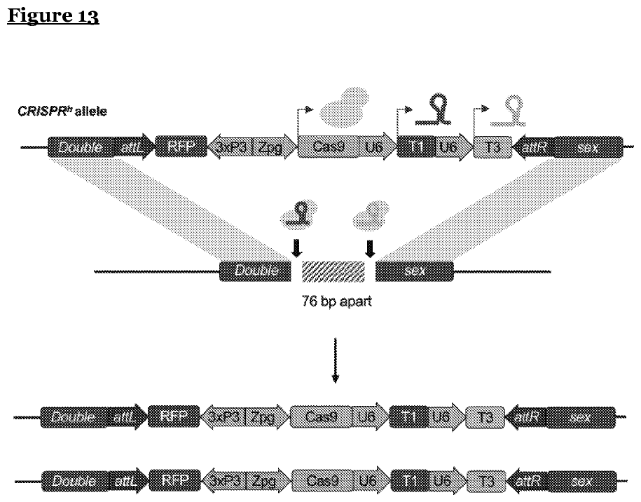

However, as described in the Examples and as shown in FIG. 13, preferably the gene drive genetic construct of the invention targets T1 and T3, which has been shown to be very effective.

Accordingly, in this embodiment in which the genetic construct is a CRISPR-based gene drive genetic construct, the construct comprises: (i) a first nucleotide sequence encoding a first guide RNA which is capable of hybridising to a first target site which is an intron-exon boundary of the female specific splice form of the doublesex (dsx) gene, and (ii) a fifth nucleotide sequence encoding a second guide RNA which is capable of hybridising to a second target site disposed in exon 5 of the female specific splice form of the doublesex (dsx) gene.

Preferably, the first and/or fifth nucleotide sequence encodes a guide RNA, most preferably separate guide RNA molecules. Preferably, each guide RNA is at least 16 base pairs in length. Preferably, each guide RNA is between 16 and 30 base pairs in length, more preferably between 18 and 25 base pairs in length.

As discussed herein, the second nucleotide sequence encodes a CRISPR nuclease, preferably a Cpf1 or Cas9 nuclease, most preferably a Cas9 nuclease, though other nuclease are known in the art.

The first, second and fifth nucleotide sequences may be on separate nucleic acid molecules. Preferably, however, the first, second and fifth nucleotide sequences are on, or form part of, the same nucleic acid molecule. Most preferably, the first, second and fifth nucleotide sequences are expressed separately. Preferably, the first nucleotide sequence is disposed 5′ of the fifth nucleotide sequence. Preferably, the second nucleotide sequence encoding the nuclease is disposed 5′ of the first and fifth nucleotide sequences.

In one embodiment, the fifth nucleotide sequence encoding a nucleotide sequence that is capable of hybridising to the second target site (i.e. T2 shown in FIG. 12) disposed in exon 5 of the female specific splice form of the doublesex (dsx) gene (i.e. the second guide RNA component) is provided herein as SEQ ID No: 39, as follows:

[SEQ ID No: 39]
TCTGAACATGTTTGATGGCG

Accordingly, preferably the fifth nucleotide sequence encoding a nucleotide sequence that is capable of hybridising to the second target site comprises a nucleic acid sequence substantially as set out in SEQ ID NO: 39, or a fragment or variant thereof.

In another embodiment, the fifth nucleotide sequence encoding a nucleotide sequence that is capable of hybridising to the second target site (i.e. T3 shown in FIG. 12) disposed in exon 5 of the female specific splice form of the doublesex (dsx) gene (i.e. the second guide RNA component) is provided herein as SEQ ID No: 40, as follows:

[SEQ ID No: 40]
GCAATACCACCCGTCAGAG

Accordingly, preferably the fifth nucleotide sequence encoding a nucleotide sequence that is capable of hybridising to the second target site comprises a nucleic acid sequence substantially as set out in SEQ ID NO: 40, or a fragment or variant thereof.

In yet another embodiment, the fifth nucleotide sequence encoding a nucleotide sequence that is capable of hybridising to the second target site (i.e. T4 shown in FIG. 12) disposed in exon 5 of the female specific splice form of the doublesex (dsx) gene (i.e. the second guide RNA component) is provided herein as SEQ ID No: 41, as follows:

[SEQ ID No: 41]
GTTTATCATCCACTCTGA

Accordingly, preferably the fifth nucleotide sequence encoding a nucleotide sequence that is capable of hybridising to the second target site comprises a nucleic acid sequence substantially as set out in SEQ ID NO: 41, or a fragment or variant thereof.

The skilled person would understand that the nucleotide sequence (i.e. guide RNA) that is capable of hybridising to the second target site in the doublesex (dsx) gene may further comprise a CRISPR nuclease binding sequence, preferably a Cpf1 or Cas9 nuclease binding sequence, and most preferably a Cas9 nuclease binding sequence. The CRISPR nuclease binding sequence creates a secondary binding structure which complexes with the nuclease, for example a hairpin loop.

Accordingly, in one preferred embodiment, the second nucleotide sequence encoding a nucleotide sequence that is capable of hybridising to the second target site (i.e. the second guide RNA targeting T2) is provided herein as SEQ ID No: 42, as follows:

[SEQ ID No: 42]
TCTGAACATGTTTGATGGCGgttttagagctagaaatagcaagttaaaa
taaggctagtccgttatcaacttgaaaaagtggcaccgagtcggtgct

Accordingly, preferably the fifth nucleotide sequence encoding a nucleotide sequence that is capable of hybridising to the second target sequence comprises or consists of a nucleic acid sequence substantially as set out in SEQ ID NO: 42, or a fragment or variant thereof.

In another preferred embodiment, the second nucleotide sequence encoding a nucleotide sequence that is capable of hybridising to the second target site (i.e. the second guide RNA targeting T3) is provided herein as SEQ ID No: 43, as follows:

[SEQ ID No: 43]
GCAATACCACCCGTCAGAGgttttagagctagaaatagcaagttaaaat
aaggctagtccgttatcaacttgaaaaagtggcaccgagtcggtgct

Accordingly, preferably the fifth nucleotide sequence encoding a nucleotide sequence that is capable of hybridising to the second target sequence comprises or consists of a nucleic acid sequence substantially as set out in SEQ ID NO: 43, or a fragment or variant thereof.

In a further preferred embodiment, the second nucleotide sequence encoding a nucleotide sequence that is capable of hybridising to the second target site (i.e. the second guide RNA targeting T4) is provided herein as SEQ ID No: 44, as follows:

[SEQ ID No: 44]
GTTTATCATCCACTCTGAgttttagagctagaaatagcaagttaaaata
aggctagtccgttatcaacttgaaaaagtggcaccgagtcggtgct

Accordingly, preferably the fifth nucleotide sequence encoding a nucleotide sequence that is capable of hybridising to the second target sequence comprises or consists of a nucleic acid sequence substantially as set out in SEQ ID NO: 44, or a fragment or variant thereof.

In one embodiment, the nucleotide sequence which is encoded by the fifth nucleotide sequence and which is capable of hybridising to the second target site (i.e. the second guide RNA targeting T2 component) is provided herein as SEQ ID No: 59, as follows:

[SEQ ID No: 59]
UCUGAACAUGUUUGAUGGCG

Accordingly, preferably the nucleotide sequence which is encoded by the fifth nucleotide sequence and which is capable of hybridising to the second target site (i.e. the second guide RNA targeting T2) comprises nucleic acid sequence substantially as set out in SEQ ID NO: 59, or a fragment or variant thereof.

In one embodiment, the nucleotide sequence which is encoded by the fifth nucleotide sequence and which is capable of hybridising to the second target site (i.e. the second guide RNA targeting T2) is provided herein as SEQ ID No: 45, as follows:

[SEQ ID No: 45]
UCUGAACAUGUUUGAUGGCGGUUUUAGAGCUAGAAAUAGCAAGUUAAAA
UAAGGCUAGUCCGUUAUCAACUUGAAAAAGUGGCACCGAGUCGGUGCU

Accordingly, preferably the nucleotide sequence which is encoded by the fifth nucleotide sequence and which is capable of hybridising to the second target site (i.e. the second guide RNA targeting T2) comprises or consists of a nucleic acid sequence substantially as set out in SEQ ID NO: 45, or a fragment or variant thereof.

In another embodiment, the nucleotide sequence which is encoded by the fifth nucleotide sequence and which is capable of hybridising to the second target site (i.e. the second guide RNA targeting T3 component) is provided herein as SEQ ID No: 60, as follows:

[SEQ ID No: 60]
GCAAUACCACCCGUCAGAG

Accordingly, preferably the nucleotide sequence which is encoded by the fifth nucleotide sequence and which is capable of hybridising to the second target site (i.e. the second guide RNA targeting T3) comprises nucleic acid sequence substantially as set out in SEQ ID NO: 60, or a fragment or variant thereof.

In another embodiment, the nucleotide sequence which is encoded by the fifth nucleotide sequence and which is capable of hybridising to the second target site (i.e. the second guide RNA targeting T3) is provided herein as SEQ ID No: 46, as follows:

[SEQ ID No: 46]
GCAAUACCACCCGUCAGAGGUUUUAGAGCUAGAAAUAGCAAGUUAAAAU
AAGGCUAGUCCGUUAUCAACUUGAAAAAGUGGCACCGAGUCGGUGCU

Accordingly, preferably the nucleotide sequence which is encoded by the fifth nucleotide sequence and which is capable of hybridising to the second target site (i.e. the second guide RNA targeting T3) comprises or consists of a nucleic acid sequence substantially as set out in SEQ ID NO: 46, or a fragment or variant thereof.

In a further embodiment, the nucleotide sequence which is encoded by the fifth nucleotide sequence and which is capable of hybridising to the second target site (i.e. the second guide RNA targeting T4 component) is provided herein as SEQ ID No: 61, as follows:

[SEQ ID No: 61]
GUUUAUCAUCCACUCUGA

Accordingly, preferably the nucleotide sequence which is encoded by the fifth nucleotide sequence and which is capable of hybridising to the second target site (i.e. the second guide RNA targeting T4) comprises a nucleic acid sequence substantially as set out in SEQ ID NO: 61, or a fragment or variant thereof.

In a further embodiment, the nucleotide sequence which is encoded by the fifth nucleotide sequence and which is capable of hybridising to the second target site (i.e. the second guide RNA targeting T4) is provided herein as SEQ ID No: 47, as follows:

[SEQ ID No: 47]
GUUUAUCAUCCACUCUGAGUUUUAGAGCUAGAAAUAGCAAGUUAAAAUA
AGGCUAGUCCGUUAUCAACUUGAAAAAGUGGCACCGAGUCGGUGCU

Accordingly, preferably the nucleotide sequence which is encoded by the fifth nucleotide sequence and which is capable of hybridising to the second target site (i.e. the second guide RNA targeting T4) comprises or consists of a nucleic acid sequence substantially as set out in SEQ ID NO: 47, or a fragment or variant thereof.

The CRISPR-based gene drive genetic construct further comprises at least one promoter sequence, such that expression of the first, second and fifth nucleotide sequence is under the control of the same promoter.

In a preferred embodiment, however, the gene drive genetic construct comprises more than one promoter sequence, such that expression of the first, second and fifth nucleotide sequences are under the control of separate promoters. Preferably, the construct comprises a first promoter sequence operably linked to the first nucleotide sequence, a second promoter sequence operably linked to the second nucleotide sequence, and a third promoter sequence operably linked to the fifth nucleotide sequence.

The first, second and third promoter sequence may be any promoter sequence that is suitable for expression in an arthropod, and which would be known to those skilled in the art. Accordingly, the first guide RNA for targeting the first target site is expressed under control of the first promoter, the nuclease is expressed under control of the second promoter, and the second guide RNA for targeting the second target site (either T2, T3 or T4) is expressed under the control of the third promoter. Accordingly, in use, the first guide RNA targets the T1 target site, and the second guide RNA targets one or more of T2, T3 and/or T4, as described above.

Preferably, the first and/or third promoter sequence is a polymerase III promoter, and most preferably a polymerase III promoter which does not add a 5′cap or a 3′polyA tail. More preferably, the first and/or third promoter is a U6 promoter, for example as shown in SEQ ID No:49, as described herein. Preferably, the first promoter is a U6 promoter and the third promoter is a U6 promoter. In other words, preferably expression of the two guide RNAs is achieved using two separate transcription units, each one preferably containing a U6 promoter.

Preferably, the second promoter sequence is a promoter sequence that substantially restricts expression of the second nucleotide sequence to germline cells of the arthropod. For example, the second promoter sequence may be selected from a group consisting of: zpg (SEQ ID No: 7); nos (SEQ ID No: 8); exu (SEQ ID No: 9); and vasa2 (SEQ ID No: 10), as described herein. Most preferably, the second promoter is zpg (SEQ ID No: 7).

Preferably, when transcribed, the first nucleotide sequence, which encodes a nucleotide sequence (i.e. the first guide RNA) which hybridises to the first target site of the doublesex gene (i.e. T1 in FIG. 12), targets the nuclease to the first target site. Preferably, the nuclease then cleaves the doublesex gene at the first target site, such that the gene drive construct is integrated into the disrupted first target site via homology-directed repair. In addition, when transcribed, the fifth nucleotide sequence, which encodes a nucleotide sequence (i.e. the second guide RNA) which hybridises to the second target site of the doublesex gene (i.e. T2, T3 or T4), targets the nuclease to the second target site. Preferably, the nuclease then cleaves the doublesex gene at the second target site, wherein the gene drive construct is integrated into the disrupted second target site via homology-directed repair. Preferably, when both the first and fifth nucleotide sequences are transcribed, they encode nucleotide sequences (i.e. the first and second gRNAs) that hybridise to both the target sites, such that the doublesex gene is cleaved in two sites at once, removing a 76 bp region of exon 5, which is replaced by the CRISPR gene drive construct (for example, see FIG. 13). The skilled person would understand that once the gene drive construct is inserted into the genome of the arthropod, it will use the natural homology found at the site in which it is inserted in the genome.

Preferably, in one embodiment, the CRISPR-based gene drive is introduced into the arthropod via a docking construct, wherein the docking construct comprises integrase attachment sites, preferably attP integrase attachment sites, that are flanked by 5′ and 3′ homology arms (sixth and seventh nucleotide sequences, respectively) that are homologous to the genomic sequences flanking the two cut-sites which are disposed in exon 5 of the arthropod, such that when the docking construct is introduced into the arthropod, it is integrated into the arthropod's genome by homology directed repair.

In one preferred embodiment, therefore, the gene drive construct is inserted into the genome via recombinase-mediated cassette exchange. Accordingly, preferably, the CRISPR-based gene drive genetic construct further comprises integrase attachment sites, preferably attB integrase attachment sites, which, respectively, flank the first nucleotide sequence encoding the nucleotide sequence that is capable of hybridising to the first target site which is an intron-exon boundary of the female specific splice form of the doublesex (dsx) gene, and the fifth nucleotide sequence capable of hybridising to a second target site disposed in exon 5 of the female specific splice form of the doublesex (dsx) gene, the second nucleotide sequence encoding the nuclease, the first promoter sequence, the second promoter sequence and the third promoter sequence. Preferably, an attB site is disposed at the 5′ end, and an attB site is disposed at the 3′ end of the construct. The CRISPR-based gene drive construct is preferably inserted into the arthropod genome via recombinase-mediated cassette exchange, wherein the docking construct is exchanged for CRISPR-based gene drive construct through the action of an integrase, preferably φC31 integrase, which is introduced into the arthropod.

Preferably, the homology arms (i.e. the sixth and seventh nucleotide sequences) are at least 100 bp in length, at least 200 bp in length, at least 400 bp in length, at least 600 bp in length, at least 800 bp in length, at least 1000 bp in length at least 1200 bp in length, at least 1400 bp in length, at least 1600 bp in length, at least 1800 bp in length, at least 2000 bp in length. Preferably, the homology arms are up to 4000 bp in length, up to 3000 bp in length, up to 2000 bp in length. Preferably, the homology arms are between 100 and 4000 bp in length, more preferably between 150 and 3000 bp in length and most preferably between 200 and 2000 bp in length. Preferably, the homology arms are about 2000 bp in length.

In a preferred embodiment, the 5′ homology arm (i.e. the sixth nucleotide sequence) is provided herein as SEQ ID No: 11, as described herein. Accordingly, preferably the 5′ homology arm comprises or consists of a nucleic acid sequence substantially as set out in SEQ ID No: 11, or a variant or fragment thereof.

In a preferred embodiment, the 3′ homology arm (i.e. the seventh sequence) is provided herein as SEQ ID No: 50, as follows:

[SEQ ID No: 50]
GAGTGGATGATAAACTTTCCGCACCACTGTAACTGTCCGTATCT
TTGTATGTGGGTGTGTGTATGTGTGTTTGGTGAAACGAATTCAA
TAGTTCTGTGCTATTTTAAATCAAGCCGCGTGCGCAACTGATGC
CGATAAGTTCAAACTAGTGTTTAAGGAGTGGAGCGAGAGAGCCG
CACCACGGTACAGAAGGGCAGCAGAATGGGTCGGCAGCCTAGCT
GCACTGGTGCGGTGCGTCCGGCGTCTCGGGGGGAGGGCGAGGAA
ATTCTAGTGTTAAATCGGAGCAGCAAAAACAAAACAGTGGTCGT
CCCGTTCAAGAAACGGCCTGTACACACACACAGAAAACACTGCA
GCATGTTTGTACATAGTAGATCCTAGAGCAGGTGGTCGTTGCTC
CTCGAACGCTCTGGACGCACGGCTTCGCGCGTATTTGCGTAGCG
TTCCGCCGATCGTGGGTATTCGTACTGCCACAAGCCCGCTTTCT
CCCATGCAATCTCTGCAACCAAACCAACAAACAACAACAAAAAA
CCAATCGACAAAATGAATCACACCCCTTTTGTATCATCTGTATA
TTCTTGTTCTTTGCGTTCTTTTCTATGTGGCCCACGCCCCGGCG
GGTACGTAATTGCGTCGAAAACCCCGAAAACCCCGGCACATACA
GTGTACATACGGTTTGAGGACAACTTTGACCTGCAGCCCTTCTG
GGGTTGCCACGTGTAGCTATACTTGTGAGATCGGGCGCCGACGG
TGTAAAGCGCGAATGGCCGCCACACAGTGTGTCCACTCCAACAC
TACCCCTCTGGAACTACCCCGTCCAGGGATGCACCGGCTCGGCT
CATGCCCCTGCAAAACAGTCCGGGCTCCACTGTAGTAGCTCCGG
CGTTGCTCTGAGAGAAGGATGCCCTTCGAAGTGTCGAAAGCGTG
CATTGGGCGTTCAAGTGTGTGTGTGTGTGTTAGGTTTAGCGAGA
AACAGCAGCAGTTGCGTGTGCTGAAAAGCGAAGGAGTAATAGAG
TGCATAATGAAAATGAAAATGAAAATGAAGCAAAAGTAGAAGGC
GGAGGAGAGCAACCTGTGTTCCACTAGTAGCGAATAGTTTAGTC
TAGTTTCGTCACCAATCAACCTTCCAACCATCGTTCAACCAATA
CCTGAGTCAACATCGTCATCGTTATCGTGCCACAACTTTATTAA
AAATGAACCTTGTCCGCGCCACCGTAGGGTGATCTAAGGCGACC
TTTCTTACGGGCGCGACCCACATGCCATCGTCACCTTCTCCAAT
CAAAACCAACAGCCTGTACCGATGGTGTGCAATTGTGCGTGCGT
GTGTGTTATTAGCAAAAAAAGAGAAAGAGTCGACGAGAGAGAGA
TAGATCGAGATCGAGAGTACAAAAGAGCAGTAGAAATGTTCGTT
GTTTGTTTTTCGTAACACAGTTGTTTAGCCAAAATGGGAATTTC
CAATAATCCCGGGGGCGGGGAAATGCGGGAATACTGCGTACACA
CATACATCAATCAAAAAGAAAAATCCTTGCGCTACATCACTACC
GTTTGCGCGGTGCTGATCTAGAGCAGACCACTTTCCACTCCACT
CTACAATCAATCAATCTGTGCAGAAGGTATGGTAAGACGGCCTT
TGAGCGAGTCACGGTCGCCACCATAACGCCGTCCGACGAGGGCT
GAATGCGAACTTTGCTAATCGATTTTCCGCTTTCTTTTTATCCC
ACCTCCTTTTCTCTCCCTCTCTCTCTTTTGCACTGCCCCTTGTA
ACCCCCAAAAAGGTAAACGACACATTAAGACCTACGAAGCGTTG
GTGAAGTCATCGCTCGATCCGAACAGCGACCGGCTGACGGAGGA
CGACGACGAGGACGAGAACATCTCGGTGACCCGCACC

Accordingly, preferably the 3′ homology arm used in this embodiment comprises or consists of a nucleic acid sequence substantially as set out in SEQ ID No: 50, or a variant or fragment thereof.

In another preferred embodiment, however, the CRISPR-based gene drive construct may be inserted into the genome by homology directed repair, i.e. without the use of a docking construct. Accordingly, preferably, the CRISPR-based gene drive genetic construct further comprises of the two homology arms noted above, sixth and seventh nucleotide sequences, which, respectively, flank the first nucleotide sequence encoding the nucleotide sequence that is capable of hybridising to the intron-exon boundary of the doublesex (dsx) gene (i.e. the first gRNA), the fifth nucleotide sequence encoding the nucleotide sequence that is capable of hybridising to the second target site in exon 5 of the doublesex (dsx) gene (i.e. the second gRNA), the second nucleotide sequence encoding the nuclease, the first promoter sequence and the second and third promoter sequence, wherein the sixth and seventh nucleotides are homologous to the genomic sequences flanking upstream of the first target site and downstream of the second target site (preferably T3 shown in FIG. 12), such that the gene drive construct is integrated into the genome via homology-directed repair.

Preferably, the homology arms (i.e. the sixth and seventh nucleotide sequences) are at least 100 bp in length, at least 200 bp in length, at least 400 bp in length, at least 600 bp in length, at least 800 bp in length, at least 1000 bp in length at least 1200 bp in length at least 1400 bp in length, at least 1600 bp in length, at least 1800 bp in length, at least 2000 bp in length. Preferably, the third and fourth nucleotide sequences are up to 4000 bp in length, up to 3000 bp in length, up to 200 bp in length. Preferably, the third and fourth nucleotide sequences are between 100 and 4000 bp in length, more preferably between 150 and 3000 bp in length and most preferably between 200 and 2000 bp in length. Preferably, the third and fourth nucleotide sequences are about 2000 bp in length.

Accordingly, preferably the sixth nucleotide sequence comprises or consists of a nucleic acid sequence substantially as set out in SEQ ID No: 11, or a variant or fragment thereof.

Accordingly, preferably the seventh nucleotide sequence comprises or consists of a nucleic acid sequence substantially as set out in SEQ ID No: 50, or a variant or fragment thereof.

Preferably, the CRISPR-based gene drive construct targets the intron-4-exon 5 boundary of the doublesex gene (i.e. the first target site) and one of T2, T3 and/or T4 (i.e. the second target site). Most preferably, the CRISPR-based gene drive construct targets the intron-4-exon 5 boundary of the doublesex gene (i.e. the first target site) and T3 (i.e. the second target site)

In a preferred embodiment, the full DNA sequence of the multiplex CRISPR construct is provided herein as SEQ ID No: 51, as follows:

[SEQ ID No: 51]
tgcgggtgccagggcgtgcccttgggctccccgggcgcgtactc
cacctcacccatgcgatcgctccggaaagatacattgatgagtt
tggacaaaccacaactagaatgcagtgaaaaaaatgctttattt
gtgaaatttgtgatgctattgctttatttgtaaccattataagc
tgcaataaacaagttaacaacaacaattgcattcattttatgtt
tcaggttcagggggaggtgtgggaggttttttaaagcaagtaaa
acctctacaaatgtggtatggctgattatgatctagagtcgcgg
ccgctacaggaacaggtggtggcggccctcggtgcgctcgtact
gctccacgatggtgtagtcctcgttgtgggaggtgatgtccagc
ttggagtccacgtagtagtagccgggcagctgcacgggcttctt
ggccatgtagatggacttgaactccaccaggtagtggccgccgt
ccttcagcttcagggccttgtggatctcgcccttcagcacgccg
tcgcgggggtacaggcgctcggtggaggcctcccagcccatggt
cttcttctgcattacggggccgtcggaggggaagttcacgccga
tgaacttcaccttgtagatgaagcagccgtcctgcagggaggag
tcttgggtcacggtcaccacgccgccgtcctcgaagttcatcac
gcgctcccacttgaagccctcggggaaggacagcttcttgtagt
cggggatgtcggcggggtgcttcacgtacaccttggagccgtac
tggaactggggggacaggatgtcccaggcgaagggcagggggcc
gcccttggtcaccttcagcttcacggtgttgtggccctcgtagg
ggcggccctcgccctcgccctcgatctcgaactcgtggccgttc
acggtgccctccatgcgcaccttgaagcgcatgaactccttgat
gacgttcttggaggagcgcaccatggtggcgacctgtgggtccc
gggcccgcggtaccgtcgactctagcggtaccccgattgtttag
cttgttcagctgcgcttgtttatttgcttagctttcgcttagcg
acgtgttcactttgcttgtttgaattgaattgtcgctccgtaga
cgaagcgcctctatttatactccggcggtcgagggttcgaaatc
gataagcttggatcctaattgaattagctctaattgaattagtc
tctaattgaattagatccccgggcgagctcgaattaaccattgt
ggaccggtcagcgctggcggtggggacagctccggctgtggctg
ttcttgagagtcatOttcctgcggcacatcootctcgtcgacca
gttcagtttgctgagcgtaagcctgctgctgttcgtcctgcatc
atcgggaccatttgtacgggccatccgccaccaccaccatcacc
accgccgtccatttctaggggcatacccatcagcatctccgcgg
gcgccattggcggtggtgccaaggtgccattcgtttgttgctga
aagcaaaagaaagcaaattagtgttgtttctgctgcacacgata
gttttcgtttcttgccgctagacacaaacaacactgcatctgga
gggagaaatttgacgcctagctgtataacttacctcaaagttat
tgtccatcgtggtataatggacctaccgagcccggttacactac
acaaagcaagattatgcgacaaaatcacagcgaaaactagtaat
tttcatctatcgaaagcggccgagcagagagttgtttggtattg
caacttgacattctgctgtgggataaaccgcgacgggctaccat
ggcgcacctgtcagatggctgtcaaatttggcccggtttgcgat
atggagtgggtgaaattatatcccactcgctgatcgtgaaaata
gacacctgaaaacaataattgttgtgttaattttacattttgaa
gaacagcacaagttttgctgacaatatttaattacgtttcgtta
tcaacggcacggaaagattatctcgctgattatccctctcgctc
tctctgtctatcatgtcctggtcgttctcgcgtcaccccggata
atcgagagacgccatttttaatttgaactactacaccgacaagc
atgccgtgagctctttcaagttcttctgtccgaccaaagaaaca
gagaataccgcccggacagtgcccggagtgatcgatccatagaa
aatcgcccatcatgtgccactgaagcgaaccggcgtagcttgtt
ccgaatttccaagtgcttccccgtaacatccgcatataacaagc
agcccaacaacaaatacagcatcgagctcgagatggactataag
gaccacgacggagactacaaggatcatgatattgattacaaaga
cgatgacgataagatggccccaaagaagaagcggaaggtcggta
tccacggagtcccagcagccgacaagaagtacagcatcggcctg
gacatcggcaccaactctgtgggctgggccgtgatcaccgacga
gtacaaggtgcccagcaagaaattcaaggtgctgggcaacaccg
accggcacagcatcaagaagaacctgatcggagccctgctgttc
gacagcggcgaaacagccgaggccacccggctgaagagaaccgc
cagaagaagatacaccagacggaagaaccggatctgctatctgc
aagagatcttcagcaacgagatggccaaggtggacgacagcttc
ttccacagactggaagagtccttcctggtggaagaggataagaa
gcacgagcggcaccccatcttcggcaacatcgtggacgaggtgg
cctaccacgagaagtaccccaccatctaccacctgagaaagaaa
ctggtggacagcaccgacaaggccgacctgcggctgatctatct
ggccctggcccacatgatcaagttccggggccacttcctgatcg
agggcgacctgaaccccgacaacagcgacgtggacaagctgttc
atccagctggtgcagacctacaaccagctgttcgaggaaaaccc
catcaacgccagcggcgtggacgccaaggccatcctgtctgcca
gactgagcaagagcagacggctggaaaatctgatcgcccagctg
cccggcgagaagaagaatggcctgttcggaaacctgattgccct
gagcctgggcctgacccccaacttcaagagcaacttcgacctgg
ccgaggatgccaaactgcagctgagcaaggacacctacgacgac
gacctggacaacctgctggcccagatcggcgaccagtacgccga
cctgtttctggccgccaagaacctgtccgacgccatcctgctga
gcgacatcctgagagtgaacaccgagatcaccaaggcccccctg
agcgcctctatgatcaagagatacgacgagcaccaccaggacct
gaccctgctgaaagctctcgtgcggcagcagctgcctgagaagt
acaaagagattttcttcgaccagagcaagaacggctacgccggc
tacattgacggcggagccagccaggaagagttctacaagttcat
caagcccatcctggaaaagatggacggcaccgaggaactgctcg
tgaagctgaacagagaggacctgctgcggaagcagcggaccttc
gacaacggcagcatcccccaccagatccacctgggagagctgca
cgccattctgcggcggcaggaagatttttacccattcctgaagg
acaaccgggaaaagatcgagaagatcctgaccttccgcatcccc
tactacgtgggccctctggccaggggaaacagcagattcgcctg
gatgaccagaaagagcgaggaaaccatcaccccctggaacttcg
aggaagtggtggacaagggcgcttccgcccagagcttcatcgag
cggatgaccaacttcgataagaacctgcccaacgagaaggtgct
gcccaagcacagcctgctgtacgagtacttcaccgtgtataacg
agctgaccaaagtgaaatacgtgaccgagggaatgagaaagccc
gccttcctgagcggcgagcagaaaaaggccatcgtggacctgct
gttcaagaccaaccggaaagtgaccgtgaagcagctgaaagagg
actacttcaagaaaatcgagtgcttcgactccgtggaaatctcc
ggcgtggaagatcggttcaacgcctccctgggcacataccacga
tctgctgaaaattatcaaggacaaggacttcctggacaatgagg
aaaacgaggacattctggaagatatcgtgctgaccctgacactg
tttgaggacagagagatgatcgaggaacggctgaaaacctatgc
ccacctgttcgacgacaaagtgatgaagcagctgaagcggcgga
gatacaccggctggggcaggctgagccggaagctgatcaacggc
atccgggacaagcagtccggcaagacaatcctggatttcctgaa
gtccgacggcttcgccaacagaaacttcatgcagctgatccacg
acgacagcctgacctttaaagaggacatccagaaagcccaggtg
tccggccagggcgatagcctgcacgagcacattgccaatctggc
cggcagccccgccattaagaagggcatcctgcagacagtgaagg
tggtggacgagctcgtgaaagtgatgggccggcacaagcccgag
aacatcgtgatcgaaatggccagagagaaccagaccacccagaa
gggacagaagaacagccgcgagagaatgaagcggatcgaagagg
gcatcaaagagctgggcagccagatcctgaaagaacaccccgtg
gaaaacacccagctgcagaacgagaagctgtacctgtactacct
gcagaatgggcgggatatgtacgtggaccaggaactggacatca
accggctgtccgactacgatgtggaccatatcgtgcctcagagc
tttctgaaggacgactccatcgacaacaaggtgctgaccagaag
cgacaagaaccggggcaagagcgacaacgtgccctccgaagagg
tcgtgaagaagatgaagaactactggcggcagctgctgaacgcc
aagctgattacccagagaaagttcgacaatctgaccaaggccga
gagaggcggcctgagcgaactggataaggccggcttcatcaaga
gacagctggtggaaacccggcagatcacaaagcacgtggcacag
atcctggactcccggatgaacactaagtacgacgagaatgacaa
gctgatccgggaagtgaaagtgatcaccctgaagtccaagctgg
tgtccgatttccggaaggatttccagttttacaaagtgcgcgag
atcaacaactaccaccacgcccacgacgcctacctgaacgccgt
cgtgggaaccgccctgatcaaaaagtaccctaagctggaaagcg
agttcgtgtacggcgactacaaggtgtacgacgtgcggaagatg
atcgccaagagcgagcaggaaatcggcaaggctaccgccaagta
cttcttctacagcaacatcatgaactttttcaagaccgagatta
ccctggccaacggcgagatccggaagcggcctctgatcgagaca
aacggcgaaaccggggagatcgtgtgggataagggccgggattt
tgccaccgtgcggaaagtgctgagcatgccccaagtgaatatcg
tgaaaaagaccgaggtgcagacaggcggcttcagcaaagagtct
atcctgcccaagaggaacagcgataagctgatcgccagaaagaa
ggactgggaccctaagaagtacggcggcttcgacagccccaccg
tggcctattctgtgctggtggtggccaaagtggaaaagggcaag
tccaagaaactgaagagtgtgaaagagctgctggggatcaccat
catggaaagaagcagcttcgagaagaatcccatcgactttctgg
aagccaagggctacaaagaagtgaaaaaggacctgatcatcaag
ctgcctaagtactccctgttcgagctggaaaacggccggaagag
aatgctggcctctgccggcgaactgcagaagggaaacgaactgg
ccctgccctccaaatatgtgaacttcctgtacctggccagccac
tatgagaagctgaagggctcccccgaggataatgagcagaaaca
gctgtttgtggaacagcacaagcactacctggacgagatcatcg
agcagatcagcgagttctccaagagagtgatcctggccgacgct
aatctggacaaagtgctgtccgcctacaacaagcaccgggataa
gcccatcagagagcaggccgagaatatcatccacctgtttaccc
tgaccaatctgggagcccctgccgccttcaagtactttgacacc
accatcgaccggaagaggtacaccagcaccaaagaggtgctgga
cgccaccctgatccaccagagcatcaccggcctgtacgagacac
ggatcgacctgtctcagctgggaggcgacaaaaggccggcggcc
acgaaaaaggccggccaggcaaaaaagaaaaagtaattaattaa
gaggacggcgagaagtaatcatatgtccgcattttgcgcaaacc
aggcgcttagacaatttgcgcgtaagcacattcgaaatgtgaaa
agctgaaagcagtggtttcgccagcccgagttcagcgaaacgga
ttccttccaagtgtttgcattcctggcggagtgttcctcccaaa
atgcactcaccctgcgtgcagtgccaaatcgtgagtttcctaat
tttttcatattgtttattacctaccaactaaagttgttgttata
tattgcgttttacgtacgacaaataagttcgtattcagaaatat
ttgcgataagagagaactcatttgcgatgaatctcattgtattt
agctaagtgccttgataagtaagcggaacagcaggaatatgaca
ctccttgggaaatacatgtaagcgtctgtaattagatatatata
cacgcaaccaaatggtccatggttgatttaagcactgcctgttg
tcgaacattgctataagcaaaataaagaagcattcattaatcta
aaatttcttcaaagtgacttcaatgatgatctctaggctatagt
gaaagctgaaagcttatttgacaatgcaagggaaagtgacgcac
gtgcgtcgtatgggaccgcgcgcatctattctctcagctaattc
ccctaatcattagtaattgacggcacgatttctgcttcttactt
ccttttactttggagcttttcatcaataaaaccagtaccatggc
cgtacgctcaacggaaaagcattcaaaaaaacccgcgttcctcg
tgtgatttgtgggtgagtggcgccatctattagagaatagctgt
actacatctcgtggacgaaggggtcagagaagttgaaagagagc
ttgatcgactgctatccaagctaggcgaggaagggagatcgcta
gagcaaaagaaaaaaaataagcaaatatctttttttataacaaa
tcgacgttagcgaaatatgtttgaatcgatttaacggttagaat
tccctttggttcgttcattatgcgaggcgcgcctttgtatgcgt
gcgcttgaagggttgatcggaaccttacaacagttgtagctata
cggctgcgtgtggcttctaacgttatccatcgctagaagtgaaa
cgaatgtgcgtaggtatatatatgaaatggagttgctctctgct
GTTTAACACAGGTCAAGCGGgttttagagctagaaatagcaagt
taaaataaggctagtccgttatcaacttgaaaaagtggcaccga
gtcggtgctttttttttttgtatgcgtgcgcttgaagggttgat
cggaaccttacaacagttgtagctatacggctgcgtgtggcttc
taacgttatccatcgctagaagtgaaacgaatgtgcgtaggtat
atatatgaaatggagttgctctctgctGCAATACCACCCGTCAG
AGgttttagagctagaaatagcaagttaaaataaggctagtccg
ttatcaacttgaaaaagtggcaccgagtcggtgcttttttttac
gcgtgggtcccatgggtgaggtggagtacgcgcccggggagccc
aagggcacgccctggcacccgca

Accordingly, preferably the gene drive construct comprises or consists of a nucleic acid sequence substantially as set out in SEQ ID NO: 51, or a fragment or variant thereof.

In a second aspect, there is provided the use of the gene drive genetic construct of the first aspect, to disrupt an intron-exon boundary of the female specific splice form of the doublesex gene in an arthropod, such that when the construct is expressed, the exon is spliced out of a doublesex precursor-mRNA transcript, wherein the female arthropod's reproductive capacity is suppressed when females are homozygous for the construct.

Preferably, the doublesex gene, the intron-exon boundary, the gene drive genetic construct, and the arthropod are as defined in the first aspect. The gene drive genetic construct may be capable of additionally targeting a second target site, which is disposed in exon 5 of the female specific splice form of the doublesex (dsx) gene, as described in relation to the first aspect. Preferably, the use comprises multiplexed genome targeting. In other words, preferably, T1 shown in FIG. 12 is targeted together with T2, T3 and/or T4, most preferably T1 and T3.

In a third aspect, there is provided a method for preventing or reducing the inclusion of at least one exon into the female specific splice form of arthropod doublesex mRNA, when said mRNA is produced by splicing from a precursor mRNA transcript, the method comprising contacting one or more cells of an arthropod, preferably one or more cells of an arthropod embryo, in vitro or ex vivo, under conditions conducive to uptake of the gene drive genetic construct of the first aspect by such a cell, and allowing splicing to take place.

Preferably, the doublesex gene, the intron-exon boundary, the gene drive genetic construct, and the arthropod are as defined in the first aspect. The gene drive genetic construct may be capable of additionally targeting a second target site, which is disposed in exon 5 of the female specific splice form of the doublesex (dsx) gene, as described in relation to the first aspect. Preferably, the method comprises multiplexed genome targeting. In other words, preferably, T1 shown in FIG. 12 is targeted together with T2, T3 and/or T4, most preferably T1 and T3.

In a fourth aspect, there is provided a method of producing a genetically modified arthropod, the method comprising introducing into an arthropod a gene drive genetic construct capable of disrupting an intron/exon boundary of the female specific splice form of the doublesex gene in an arthropod, such that when the gene-drive construct is expressed, an exon is spliced out of a doublesex precursor-mRNA transcript, wherein a female arthropod, which is homozygous for the construct, exhibits a suppressed reproductive capacity.

Preferably, the doublesex gene, the intron-exon boundary, the gene drive genetic construct, and the arthropod are as defined in the first aspect. The gene drive genetic construct may be capable of additionally targeting a second target site, which is disposed in exon 5 of the female specific splice form of the doublesex (dsx) gene, as described in relation to the first aspect. Preferably, the method comprises multiplexed genome targeting. In other words, preferably, T1 shown in FIG. 12 is targeted together with T2, T3 and/or T4, most preferably T1 and T3.

The gene drive genetic construct may be introduced directly into an arthropod host cell, preferably an arthropod host cell present in an arthropod embryo, by suitable means, e.g. direct endocytotic uptake. The construct may be introduced directly into cells of a host arthropod (e.g. a mosquito) by transfection, infection, electroporation, microinjection, cell fusion, protoplast fusion or ballistic bombardment. Alternatively, constructs of the invention may be introduced directly into a host cell using a particle gun.

Preferably, the construct is introduced into a host cell by microinjection of arthropod embryos, preferably an insect embryo and most preferably mosquito embryos.

Preferably, the gene drive genetic construct is introduced into freshly laid eggs, within 2 hours of deposition. More preferably, the gene drive genetic construct is introduced into an arthropod embryo at the start of melanisation, which the skilled person would understand takes place within 30 minutes after egg laying. Preferably, the mosquito is of the subfamily Anophelinae. Preferably, the mosquito is selected from a group consisting of: Anopheles gambiae; Anopheles coluzzi; Anopheles merus; Anopheles arabiensis; Anopheles quadriannulatus; Anopheles stephensi, Anopheles funestus and Anopheles melas.

In a fifth aspect, there is provided a genetically modified arthropod obtained or obtainable by the method of the fourth aspect.

The genetically modified arthropod may be targeted for target site T1, and one or more of target sites T2, T3 and/or T4, most preferably T1 and T3.

In a sixth aspect, there is provided a genetically modified arthropod comprising a disrupted intron-exon boundary of the female specific splice form of the doublesex gene, such that the exon is spliced out of a doublesex precursor-mRNA transcript, and wherein a female arthropod, which is homozygous for the disrupted intron-exon boundary, exhibits a suppressed reproductive capacity.

Preferably, the intron-exon boundary has been disrupted by a gene drive genetic construct as defined in the first aspect. Preferably, the doublesex gene, the intron-exon boundary, the gene drive genetic construct, and the arthropod is as defined in the first aspect. The genetically modified arthropod may be targeted for target site T1, and one or more of target sites T2, T3 and/or T4, most preferably T1 and T3.

In a seventh aspect, there is provided a method of suppressing a wild type arthropod population, the method comprising breeding a genetically modified arthropod comprising an intron-exon boundary of the female specific splice form of the doublesex gene that has been disrupted by a gene drive genetic construct, such that the exon is spliced out of a doublesex precursor-mRNA transcript, with a wild type population of the arthropod, such that when the gene drive construct is expressed in offspring of the genetically modified arthropod and wild type arthropod, it disrupts the doublesex gene contributed by the wild type population, and wherein when the offspring is a female arthropod homozygous for the disrupted intron-exon boundary, it has suppressed reproductive capacity, such that female reproductive output in the population is reduced, and the wild type arthropod population is suppressed.

Preferably, the doublesex gene, the intron-exon boundary, the gene drive genetic construct, and the arthropod is as defined in the first aspect. The gene drive genetic construct may be capable of additionally targeting a second target site, which is disposed either wholly or partially in exon 5 of the female specific splice form of the doublesex (dsx) gene, as described in relation to the first aspect. Preferably, the method comprises multiplexed genome targeting. In other words, preferably, T1 shown in FIG. 12 is targeted together with T2, T3 and/or T4, most preferably T1 and T3.

In an eighth aspect, there is provided a nucleic acid comprising or consisting of a nucleotide sequence substantially as set out as any one of SEQ ID No: 6-34, 42-48, 50-57 or a fragment or variant thereof.

In a ninth aspect, there is provided a guide RNA comprising any one of SEQ ID No:58 to 61 and a nuclease binding region.

The nuclease binding region may bind to, or complex with, a CRISPR nuclease, which may be a Cas endonuclease. For example, the nuclease binding region may bind or complex with Cas9 or Cpf1. The guide RNA may comprise trans-activating CRISPR RNA (tracrRNA) and a CRISPR RNA (crRNA). Alternatively, the guide RNA may comprise a single guide RNA (sgRNA).

In a tenth aspect, there is provided the nucleic acid according to the eighth aspect or the guide RNA of the ninth aspect, for use in a genome editing method, preferably for suppressing a wild type arthropod population.

The genome editing method or technique may be carried out in vivo, in vitro or ex vivo.

Preferably, the nucleic acid according to the eighth aspect or the guide RNA of the ninth aspect is used in the method of the seventh aspect.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-94 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, SEQ ID Nos:1 to 94.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

FIG. 1 shows targeting the female-specific isoform of doublesex. (a) Schematic representation of the male- and female-specific dsx transcripts and the gRNA sequence used to target the gene (shaded in grey). The gRNA spans the Intron4-Exon5 boundary. The proto-spacer adjacent motive (PAM) of the gRNA is highlighted in blue. The scale bar indicates a 200 bp fragment. Introns are not drawn to scale. (b) Sequence alignment of the dsx Intron4-Exon5 boundary in 6 of the species from the Anopheles gambiae complex. The sequence is highly conserved within the complex suggesting tight functional constraint at this region of the dsx gene. The gRNA used to target the gene is underlined and the PAM is highlighted in blue. (c) Schematic representation of the HDR knockout construct specifically recognising exon 5 and the corresponding target locus. (d) Diagnostic PCR using a primer set (blue arrows in panel (c)) to discriminate between the wild type and dsxF allele in homozygous (dsxF^{-/-}) heterozygous (dsxF^{+/-}) and wt individuals.

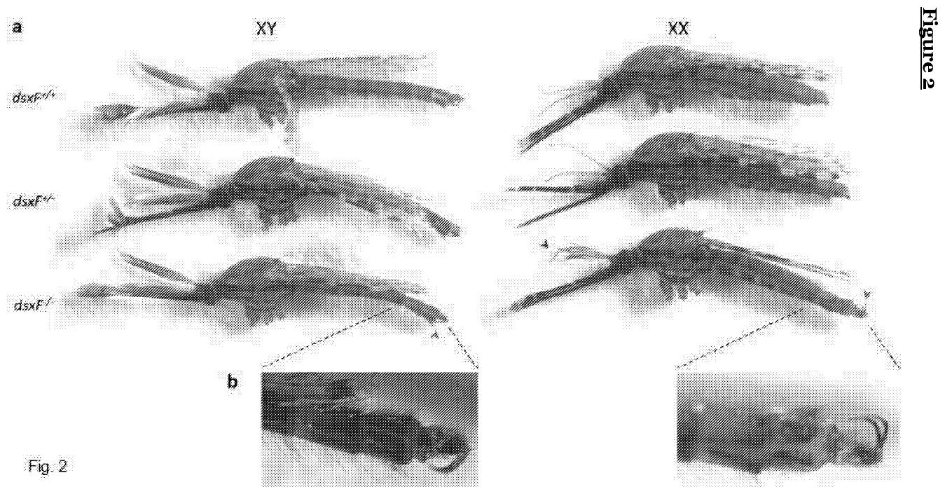

FIG. 2 shows morphological analysis of homozygous dsxF^{-/-} mutants. (a) Morphological appearance of genetic males and females heterozygous (dsxF^{+/-}) or homozygous (dsxF^{-/-}) for exon 5 null allele. This assay was performed in a strain containing dominant RFP marker linked to the Y chromosome, whose presence permits unambiguous determination of male or female genotype. Anomalies in sexual morphology were observed only in dsxF^{-/-} genetic female mosquitoes. This group of XX individuals showed male-specific traits including a plumose antenna and claspers (arrows). This group also showed anomalies in the proboscis and accordingly they could not bite and feed on blood. Representative samples of each genotype are shown. (b) Magnification of the external genitalia. All dsxF^{-/-} females carried claspers, a male-specific characteristic. The claspers were dorsally rotated rather than in the normal ventral position.

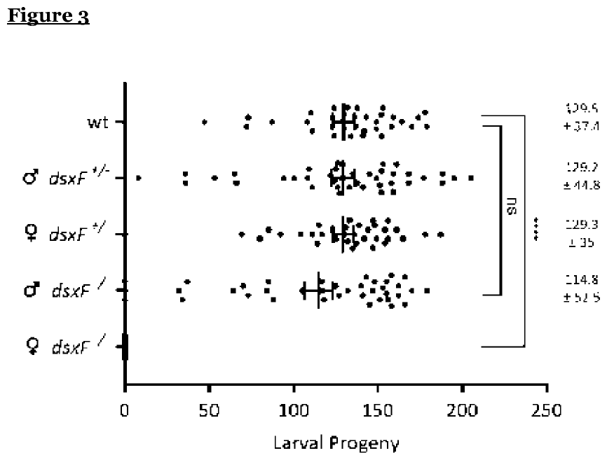

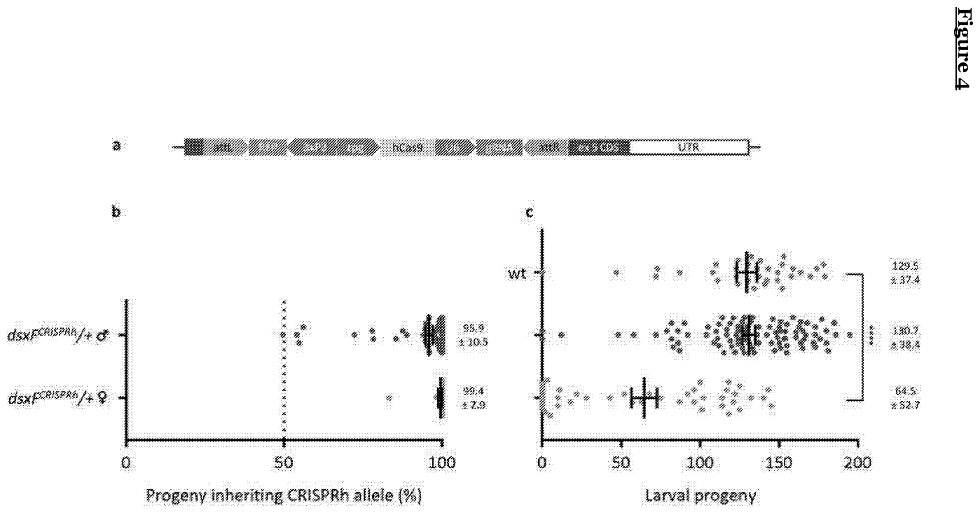

FIG. 3 shows the reproductive phenotype of dsxF mutants. Males and females dsxF^{-/-} and dsxF^{+/-} individuals were mated with the corresponding wild type sexes. Females were given access to a blood meal and subsequently allowed to lay individually. Fecundity was investigated by counting the number of larval progeny per lay (n43). Using wild type (wt) as a comparator the inventors saw no significant differences (‘ns’) in any genotype other than dsxF^{-/-} females, which were unable to feed on blood and therefore failed to produce a single egg (****, p<0.0001; Kruskal-Wallis test). Vertical bars indicate the mean and the s.e.m.

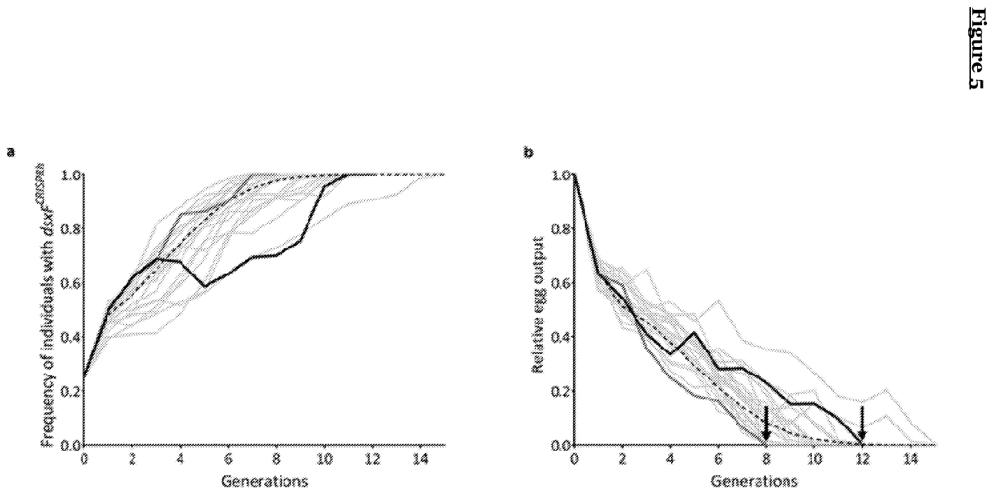

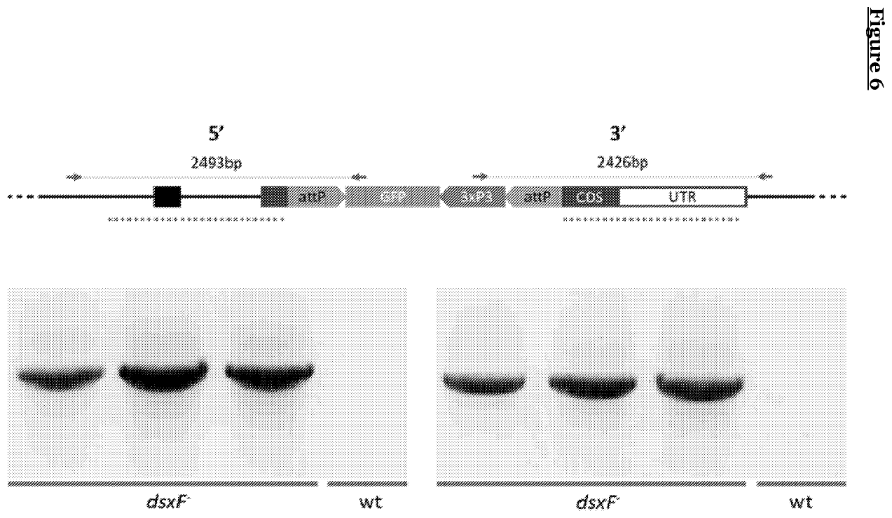

FIG. 4 shows the transmission rate of the dsxFCRISPRh driving allele and fecundity analysis of heterozygous male and female mosquitoes. Male and female mosquitoes heterozygous for the dsxFCRISPRh allele (a) (dsxFCRISPRh/+) were analysed in crosses with wild type mosquitoes to assess the inheritance bias of the dsxFCRISPRh drive construct (b) and for the effect of the construct on their reproductive phenotype (c). (b) Scattered plot of the transgenic rate observed in the progeny of dsxFCRISPRh/+ female or male mosquitoes (n≥42) crossed to wild type individuals. Each dot represents the progeny derived from single females. Both male and female dsxFCRISPRh/+ showed a high transmission rate of up to 100% of the dsxFCRISPRh allele to the progeny. The transmission rate was determined by visual scoring among offspring of the RFP marker that is linked to the dsxFCRISPRh allele. The dotted line indicates the expected Mendelian inheritance. Mean transmission rate (±s.e.m.) is shown (c) Scattered plot showing the number of larvae produced by single females from crosses of dsxFCRISPRh/+ mosquitoes with wild type individuals after one blood meal. Mean progeny count (±s.e.m.) is shown. (****, p<0.0001; Kruskal-Wallis test).

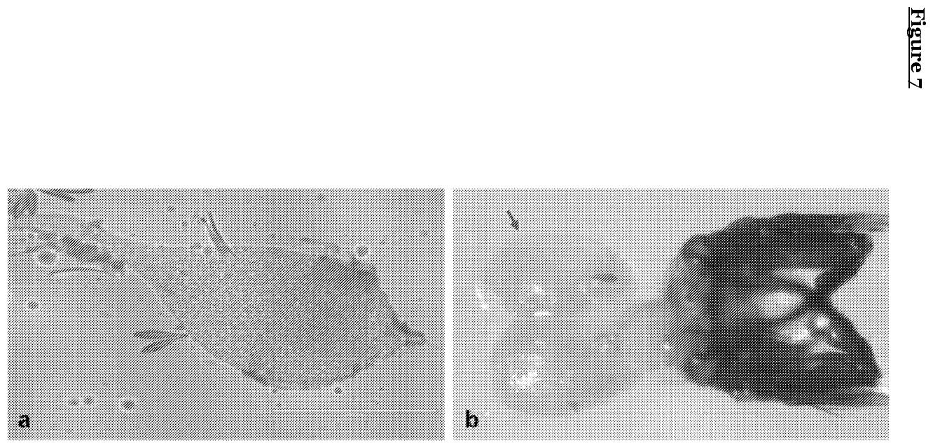

FIG. 5 shows the dynamics of the spread of the dsxF^{CRISPRh} allele and effect on population reproductive capacity. Two cages were set up with a starting population of 300 wild type females, 150 wild type males and 150 dsxF^{CRISPRh}/+ males, seeding each cage with a dsxF^{CRISPRh} allele frequency of 12.5%. The frequency of the dsxF^{CRISPRh} mosquitoes was scored for each generation (a). The drive allele reached 100% prevalence in both cage 2 (grey) and cage 1 (black) at generation 7 and 11 in agreement with a deterministic model (dotted line) that takes into account the parameter values retrieved from the fecundity assays. 20 stochastic simulations were run (light grey lines) assuming a max population size of 650 individuals. (b) Total egg output deriving from each generation of the cage was measured and normalised relative to the output from the starting generation. Suppression of the reproductive output of each cage led the population to collapse completely (black arrows) by generation 8 (cage 2) or generation 12 (cage 1). Parameter estimates included in the model are provided in Table 1.

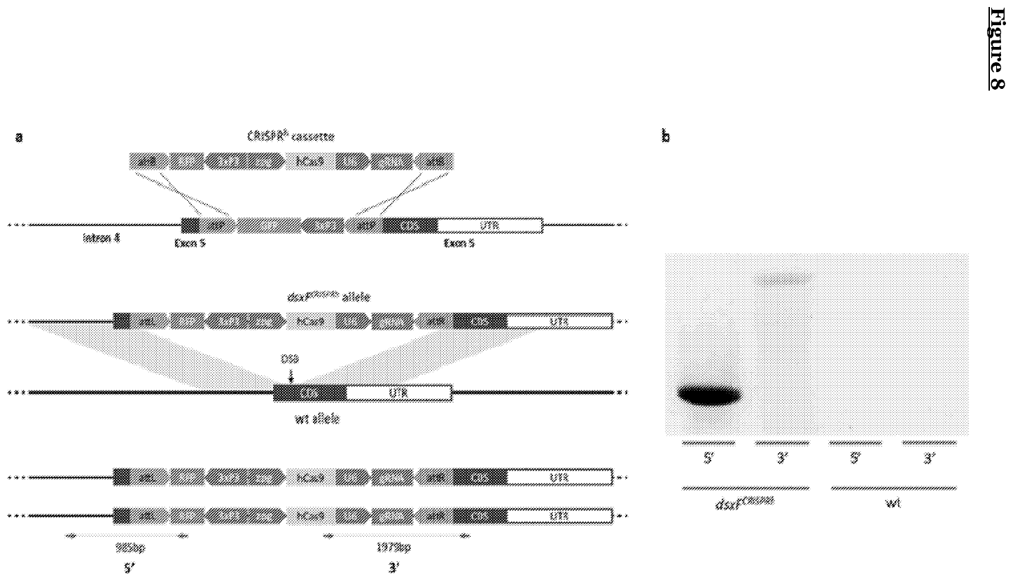

FIG. 6 shows molecular confirmation of the correct integration of the HDR-mediated event to generate dsxF−. PCRs were performed to verify the location of the dsx φC31 knock-in integration. Primers (blue arrows) were designed to bind internal of the φC31 construct and outside of the regions used for homology directed repair (HDR) (dotted gray lines) which were included in the Donor plasmid K101. Amplicons of the expected sizes should only be produced in the event of a correct HDR integration. The gel shows PCRs performed on the 5′ (left) and 3′ (right) of 3 individuals for the dsx φC31 knock-in line (dsxF⁻) and wild type (wt) as a negative control.

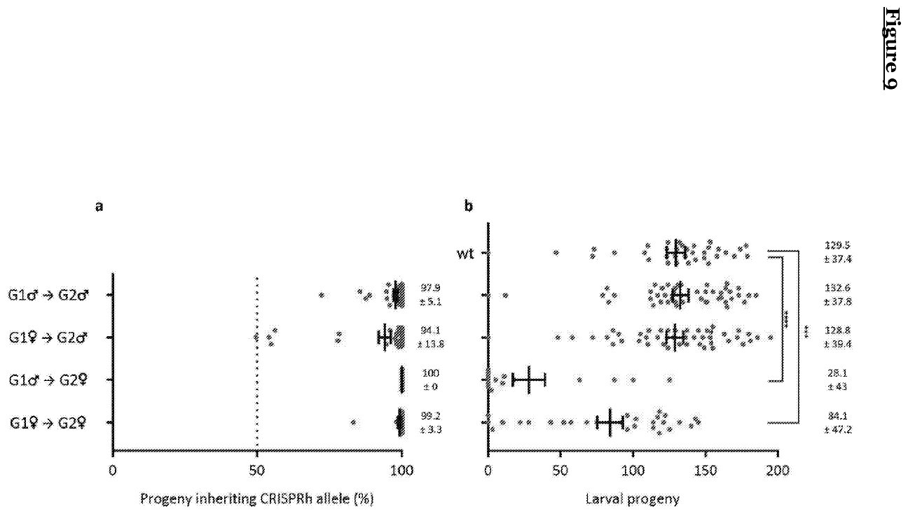

FIG. 7 shows the morphology of the dsxF^{-/-} internal reproductive organs. (a) Testis-like gonad from 3-days old female dsxF^{-/-} individual. There was no layer division between the cells and there was no evidence of sperm. (b) Dissections performed on dsxF^{-/-} genetic females revealed the presence of organs resembling accessory glands, a typical male internal reproductive organ.

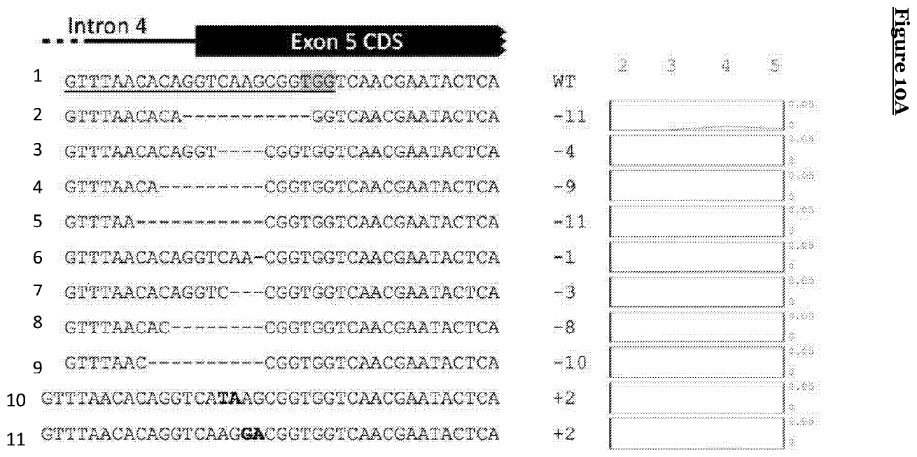

FIG. 8 shows the development of dsxF^{CRISPRh} drive construct and its predicted homing process and molecular confirmation of the locus. (a) The drive construct (CRISPRh cassette) contained the transcription unit of a human codon-optimised Cas9 controlled by the germline-restrictive zpg promoter, the RFP gene under the control of the neuronal 3×P3 promoter and the gRNA under the control of the constitutive U6 promoter, all enclosed within two attB sequences. The cassette was inserted at the target locus using recombinase-mediated cassette exchange (RMCE) by injecting embryos with a plasmid containing the cassette and a plasmid containing a $31 recombination transcription unit. During meiosis the Cas9/gRNA complex cleaves the wild type allele at the target locus (DSB) and the construct is copied across to the wild type allele via HDR (homing) disrupting exon 5 in the process. (b) Representative example of molecular confirmation of successful RMCE events. Primers (blue arrows) that bind components of the CRISPRh cassette were combined with primers that bind the genomic region surrounding the construct. PCRs were performed on both sides of the CRISPRh cassette (5′ and 3′) on many individuals as well as wild type controls (wt).

FIG. 9 shows the maternal or paternal inheritance of the dsxF^{CRISPRh} driving allele affect fecundity and transmission bias in heterozygotes. Male and female dsxF^{CRISPRh} heterozygotes (dsxF^{CRISPRh}/+) that had inherited a maternal or paternal copy of the driving allele were crossed to wild type and assessed for inheritance bias of the construct (a) and reproductive phenotype (b). (a) Progeny from single crosses (n≥15) were screened for the fraction that inherited DsRed marker gene linked to the dsxF^{CRISPRh} driving allele (e.g. G1♂→G2♀ represents a heterozygous female that received the drive allele from her father). Levels of homing were similarly high in males and females whether the allele had been inherited maternally or paternally. The dotted line indicates the expected Mendelian inheritance. Mean transmission rate (±s.e.m.) is shown. (b) Counts of hatched larvae for the individual crosses revealed a fertility cost in female dsxF^{CRISPRh} heterozygotes that was stronger when the allele was inherited paternally. Mean progeny count (±s.e.m.) is shown. (***, p<0.001;****, p<0.0001; Kruskal-Wallis test).

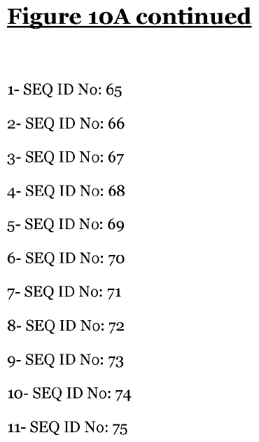

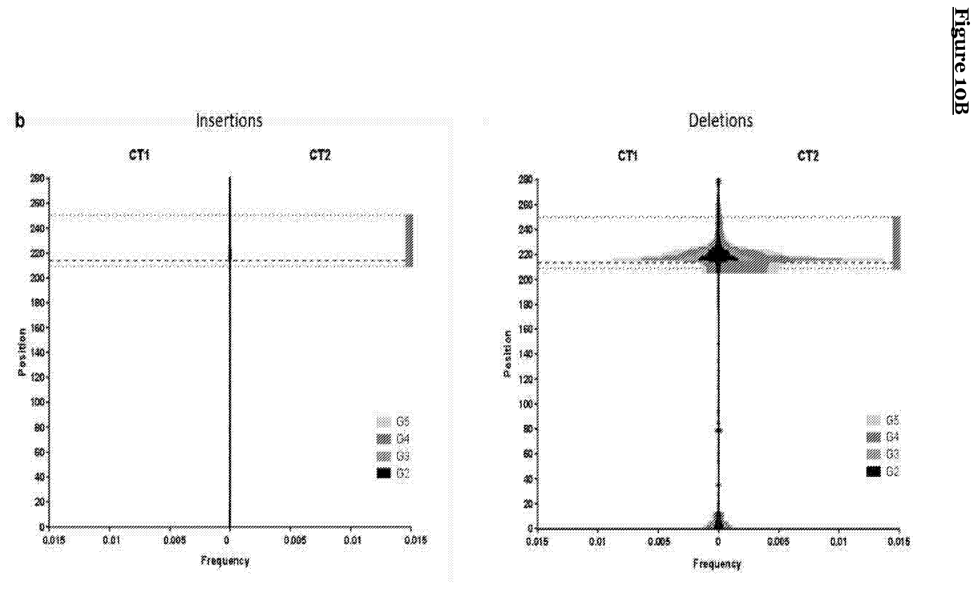

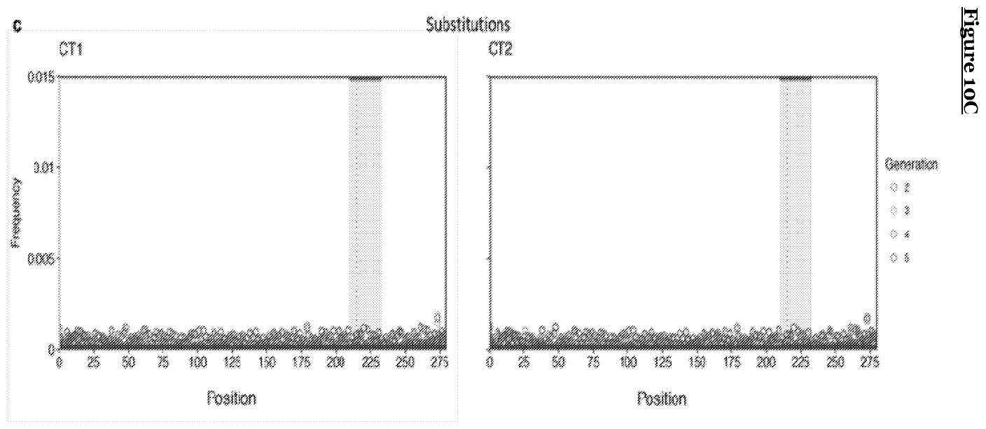

FIGS. 10A-C show resistance plots variants and deletions in sequence. Pooled amplicon sequencing of the target site from 4 generations of the cage experiment (generations 2, 3, 4 and 5) revealed a range of very low frequency indels at the target site (FIG. 10A), none of which showed any sign of positive selection. Insertion, deletion and substitution frequencies per nucleotide position were calculated, as a fraction of all non-drive alleles, from the deep sequencing analysis for both cages. Distribution of insertions and deletions (FIG. 10B) in the amplicon is shown for each cage. Contribution of insertions and deletions arising from different generations is displayed with the frequency in each generation represented by a different colour. Significant change (p<0.01) in the overall indel frequency was observed in the region around the cut-site (dotted area+/−20 bp) for both cages. No significant changes were observed in the substitution frequency (FIG. 10C) around the cut-site (shaded area+/−20 bp) when compared with the rest of the amplicon, confirming that the gene drive did not generate any substitution activity at the target locus and that the laboratory colony is devoid of any standing variation in the form of SNPs within the entire amplicon.

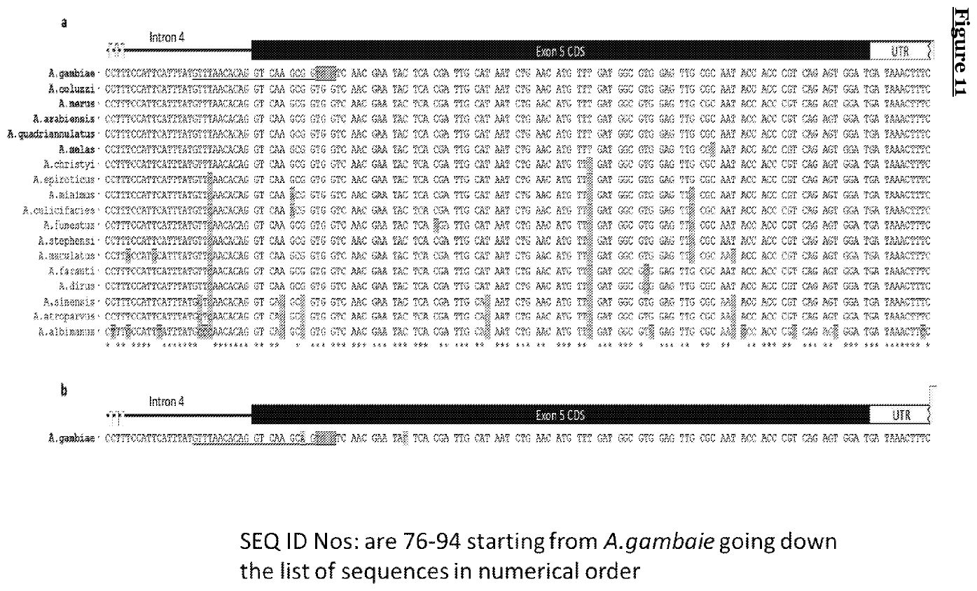

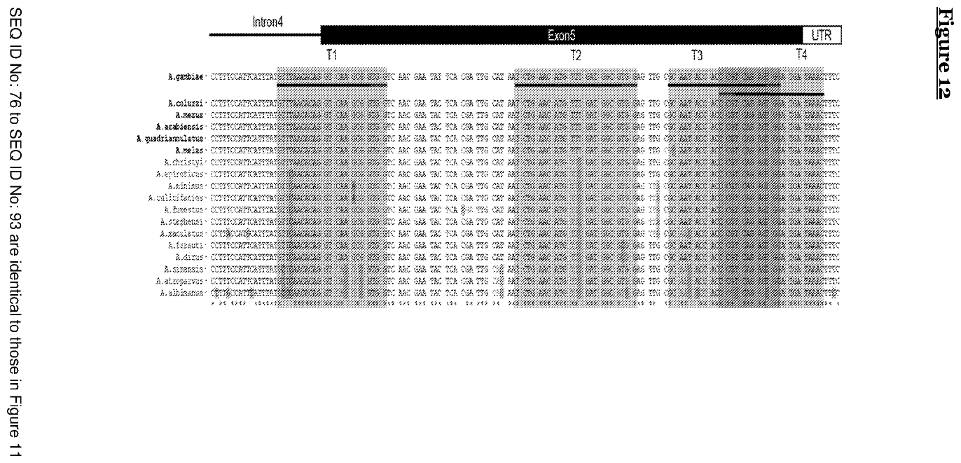

FIG. 11 shows a sequence comparison of the dsx female-specific exon 5 across members of the Anopheles genus and SNP data obtained from Anopheles gambiae mosquitoes in Africa. (a) Sequence comparison of the dsx Intron4-Exon5 boundary and the dsx female-specific exon 5 within the 16 Anopheline species. The sequence of the Intron4-Exon5 boundary is completely conserved within the six species that form the Anopheles gambiae complex (noted in bold). The gRNA used to target the gene is underlined and the PAM is highlighted in blue. Changes in the DNA sequence are shaded grey and codon silent and missense substitutions are noted in blue and red respectively. (b) SNP frequencies obtained from 765 Anopheles gambiae mosquitoes captured across Africa¹⁷. Across the dsx female-specific Exon 5 there are only 2 SNP variants (noted in yellow) with frequencies of 2.9% (the SNP in the gRNA-complementary sequence) and 0.07%.

FIG. 12 shows a sequence comparison of the dsx female-specific exon 5 across members of the Anopheles genus and SNP data obtained from Anopheles gambiae mosquitoes in Africa. It shows a further three invariant target sites (referred to as T2, T3 and T4) in addition to the original target site (referred to as T1), which have been identified in exon 5 of the Anopheles gambiae doublesex gene. A sequence alignment in the coding sequence of AgdsxF exon 5 (including part of intron 4, and the 3′ untranslated region (UTR) of exon5) amongst all available mosquito species in which a doublesex homologue could be identified is shown. Species names are shown on the left, and species in bold belong to the Anopheles gambiae species complex. Nucleotides that are variable compared to the Anopheles gambiae sensus stricto reference sequence on the top are shaded in dark grey. Nucleotides are shown in light blue or red, depending on whether a variation causes a synonymous or non-synonymous amino acid change in the exon 5 coding sequence. Asterisks denote the nucleotide positions that remained unchanged in all species. gRNA binding sites are shaded in light grey and underlined in black, the proto-spacer adjacent motives (PAMs) required for Cas9 cleavage are underlined in red. The 3′ splicing acceptor CAGG is shaded in green. In yellow, a single nucleotide polymorphism that has been identified in wild Anopheles gambiae populations, is highlighted.

FIG. 13 shows one embodiment of a novel multiplexed gene drive at doublesex. This embodiment contains a visible marker (the RFP marker), a germline-expressed Cas9 nuclease and two ubiquitously expressed gRNAs targeting target sites T1 and T3. The CRISPR construct was knocked in between the T1 and T3 cut sites. Homing analysis of the new multi-guide gene drive is shown. Promoter sequences are shown as light grey arrows.

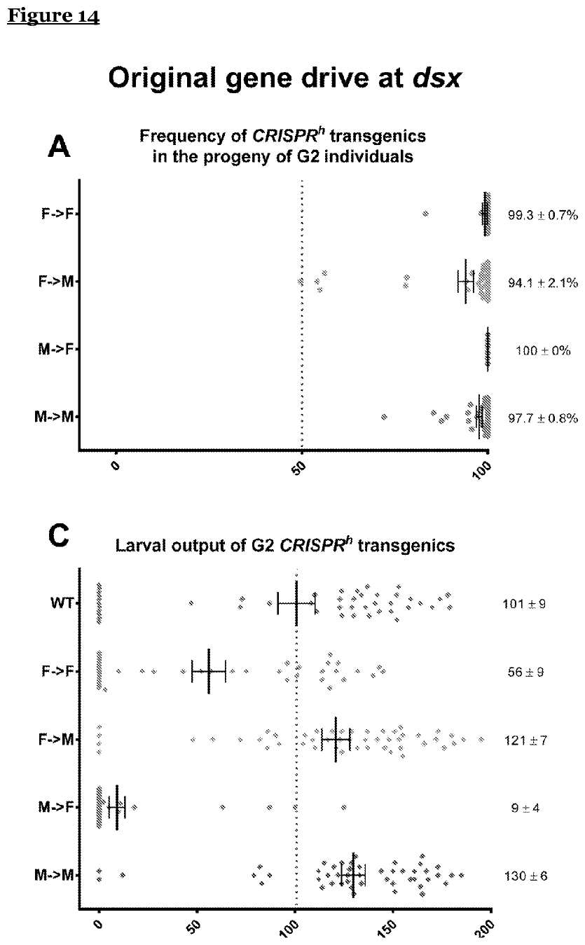

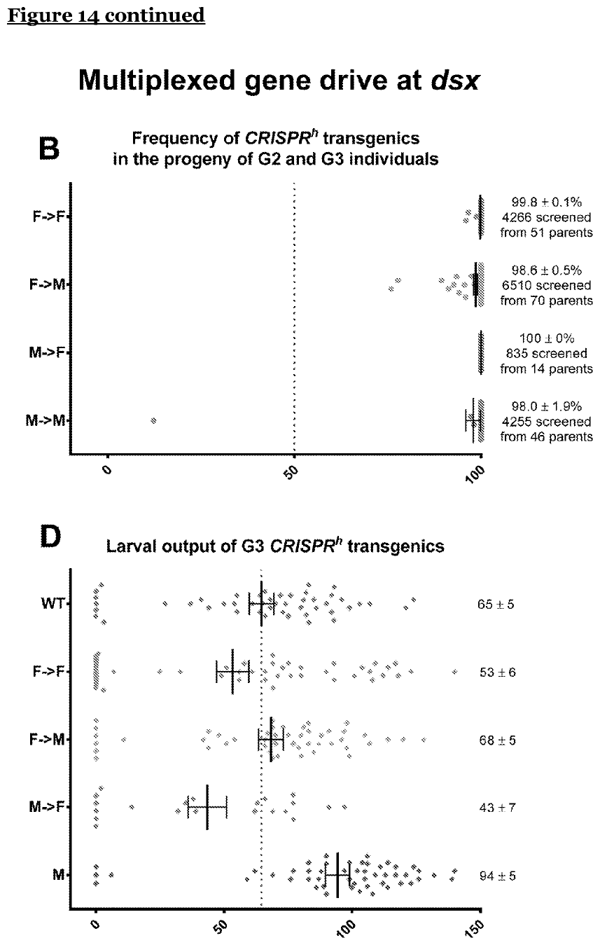

FIG. 14 shows 1a comparison of the transmission rates and fertility of heterozygous gene drive carriers when the gene drive contained a single target, i.e. T1 (FIG. 14A & C) or two targets, i.e. T1 and T3 (Figures B & D). Female or male gene drive carriers that inherited the drive from a female or male transgenic individual (F->F, F->M, M->F, M->M) were crossed to wild-type mosquitoes. Females were allowed to lay individually. The reproductive output of females was determined by counting eggs and hatched larvae and transmission rates were determined by screening the progeny for RFP fluorescence, indicative of carrying the gene drive. Figures A & B show that the transmission rates correspond to the total number of RFP+ progeny over the total number of screened progeny per female. Mean transmission rates s.e.m. (standard error of mean) are shown. Figures C & D show that the larval output of each class is shown, including a wild-type control, as the standard for comparison (red line). Mean larval outputs s.e.m. are shown. Note that females with zero larval output that showed no evidence of mating were all included in the analysis, since mating competence can be affected by carrying mutations at doublesex. The results from Kyrou et al. (2018) shown on the left were adapted to also include unmated individuals in the analysis.

### EXAMPLES

The invention described herein relies on inserting site-specific nuclease genes into a locus of choice, in formations that both confer some trait of interest on an individual and lead to a biased inheritance of the trait. The approach relies on “homing” leading to suppression. The invention is focused on population suppression, whereby the gene drive construct is designed to insert within a target gene in such a way that the gene product, or a specific isoform thereof, is disrupted. To build the nuclease-based gene drive of the invention, the nuclease gene is inserted within its own recognition sequence in the genome such that a chromosome containing the nuclease gene cannot be cut, but chromosomes lacking it are cut. When an individual contains both a nuclease-carrying chromosome and an unmodified chromosome (i.e. heterozygous for the gene drive), the unmodified chromosome is cut by the nuclease. The broken chromosome is usually repaired using the nuclease-containing chromosome as a template and, by the process of homologous recombination, the nuclease is copied into the targeted chromosome. If this process, called “homing”, is allowed to proceed in the germline, then it results in a biased inheritance of the nuclease gene, and its associated disruption, because sperm or eggs produced in the germline can inherit the gene from either the original nuclease-carrying chromosome, or the newly modified chromosome.

Due to the negative reproductive load the gene drive imposes, selection can be expected to occur for resistant alleles. The most likely source of such resistance is sequence variation at the target site that prevents the nuclease cutting yet at the same time permits a functional product from the target gene. Such variation can pre-exist in a population or can be created by activity of the nuclease itself—a small proportion of cut chromosomes, rather than using the homologous chromosome as a template, can instead be repaired by end-joining (EJ), which can introduce small insertions or deletions (“indels”) or base substitutions during the repair of the target site. In-frame indels or conservative substitutions might be expected to show selection in the presence of a gene drive. The inventors have previously observed target site resistance in cage experiments (data not shown) and found that end-joining in chromosomes of the early embryo, due to parentally-deposited nuclease, was likely to be the predominant source of the resistant alleles at the target site.

In mitigating and preventing the emergence of resistant alleles, the strategy being investigated by the inventors involves carefully selecting target sites in regions of the target gene that are so functionally constrained and conserved that most variation is unlikely to restore function to the gene, meaning that the majority of variants will simply not confer any selective advantage. The inventors therefore investigated whether Anopheles gambiae doublesex gene (dsx) is a suitable target for a gene drive approach aimed at suppressing population reproductive capacity to eradicate malaria. To do this, they disrupted the intron 4-exon 5 boundary of dsx (referred to as target site “T1”) with the primary objective to prevent the formation of functional AgdsxF while leaving the AgdsxM transcript unaffected. They also disrupted target sites (referred to as T2, T3 and T4) in addition to the original target site, T1.

### Materials and Methods

### Population Genetics Model

To model the results of the cage experiments, the inventors used discrete-generation recursion equations for the genotype frequencies, treating males and females separately. F_ij (t) and M_ij (t) denote the frequency of females (or males) of genotype i/j in the total female (or male) population. The inventors considered three alleles, W (wildtype), D (driver) and R (non-functional resistant), and therefore six genotypes.

### Homing

Adults of genotype W/D produce gametes at meiosis in the ratio W:D:R as follows:

(1−d_{f})(1−u_{f});d_{f}:(1−d_{f})u_{f} in females

(1−dₘ)(1−uₘ);dₘ:(1−dₘ)uₘ in males

Here, d_f and d_m are the rates of transmission of the driver allele in the two sexes and u_f and u_m are the fractions of non-drive gametes that are non-functional resistant (R alleles) from meiotic end-joining. In all other genotypes, inheritance is Mendelian. Fitness. Let w_ij≤1 represent the fitness of genotype i/j relative to w_WW=1 for the wild type homozygote. The inventors assume no fitness effects in males. Fitness effects in females are manifested as differences in the relative ability of genotypes to participate in mating and reproduction. The inventors assume the target gene is needed for female fertility, thus D/D, D/R and R/R females are sterile; there is no reduction in fitness in females with only one copy of the target gene (W/D, W/R).

### Parental Effects

The inventors consider that further cleavage of the W allele and repair can occur in the embryo if nuclease is present, due to one or both contributing gametes derived from a parent with one or two driver alleles. The presence of parental nuclease is assumed to affect somatic cells and therefore female fitness but has no effect in germline cells that would alter gene transmission. Previously, embryonic EJ effects (maternal only) were modelled as acting immediately in the zygote [1,2]. Here, the inventors consider that experimental measurements of female individuals of different genotypes and origins show a range of fitnesses, suggesting that individuals may be mosaics with intermediate phenotypes. The inventors therefore model genotypes W/X (X=W, D, R) with parental nuclease as individuals with an intermediate reduced fitness w_{WX}¹⁰, w_{WX}¹⁰, or w_{WX}¹¹ depending on whether nuclease was derived from a transgenic mother, father, or both. The inventors assume that parental effects are the same whether the parent(s) had one or two drive alleles. For simplicity, a baseline reduced fitness of w₁₀, w₀₁, w₁₁ is assigned to all genotypes W/X (X=W, D, R) with maternal, paternal and maternal/paternal effects, with fitness estimated as the product of mean egg production values and hatching rates relative to wild type in Table 1 in the deterministic model. In the stochastic version of the model, egg production from female individuals with different parentage is sampled with replacement from experimental values.

TABLE 1
Parameters for stochastic cage model

| | | Method of |
|---|---|---|
| Parameter | Estimate | estimation |
| Mating | 0.85 for heterozygotes; 0 for | Estimated |
| probability | D/D, D/R and R/R homozygotes | from Hammond |
| | | et al. 2017 |
| Egg production | Mean 137.4. Sampling with | From assays |
| from wildtype | replacement of observed values | of mated |
| female (no | (10, 61, 96, 98, 111, 111, 113, | females |
| parental | 127, 128, 129, 132, 132, 134, | |
| nuclease) | 135, 137, 138, 138, 139, 142, | |
| | 142, 146, 146, 149, 152, 152, | |
| | 152, 158, 160, 162, 164, 170, | |
| | 179, 186, 189, 191) | |
| Egg production | Mean 118.96. Sampling with | From assays |
| from W/D | replacement of observed values | of mated |
| heterozygote | (12, 31, 76, 90, 96, 100, 106, | females |
| female (nuclease | 106, 107, 113, 117, 118, 119, | |
| from ♀) | 130, 133, 136, 136, 136, 137, | |
| | 138, 139, 142, 143, 145, 146, | |
| | 148, 157, 174) | |
| Egg production | Mean 59.67. Sampling with | From assays |
| from W/D | replacement of observed values | of mated |
| heterozygote | (0, 0, 0, 0, 0, 34, 47, 50, 65, | females |
| female (nuclease | 105, 113, 115, 115, 125, 126) | |
| from ♂) | | |
| Hatching | 0.941 | From assays |
| probability, | | of mated |
| wildtype female | | females |
| (no parental | | |
| nuclease) | | |
| Hatching | 0.707 | From assays |
| probability, | | of mated |
| W/D heterozygote | | females |
| female (nuclease | | |
| from ♀) | | |
| Hatching | 0.47 | From assays |
| probability, | | of mated |
| W/D heterozygote | | females |
| female (nuclease | | |
| from ♂) | | |
| Probability | 0.8708 | Average of |
| of emergence | | observations |
| from pupa | | over all |
| (survival | | generations |
| from larva) | | and both cage |
| | | experiments |
| Drive in | 0.9985 | Observed |
| W/D females | | fraction |
| | | transgenic |
| | | from assays |
| Drive in | 0.9635 | Observed |
| W/D males | | fraction |
| | | transgenic |
| | | from assays |
| Meiotic EJ | 0.4685 | Estimated |
| parameter | | from Hammond |
| (fraction | | et al. 2016 |
| non-drive | | |
| alleles that | | |
| are resistant) | | |

### Recursion Equations

The inventors firstly considered the gamete contributions from each genotype, including parental effects on fitness. In addition to W and R gametes that are derived from parents that have no drive allele and therefore have no deposited nuclease, gametes from W/D females and W/D, D/R and D/D males carry nuclease that is transmitted to the zygote, and these are denoted as W\hat{ }*, D\hat{ }*, R\hat{ }*. The proportion of type i alleles in eggs produced by females participating in reproduction are given in terms of male and female genotype frequencies below. Frequencies of mosaic individuals with parental effects (i.e., reduced fitness) due to nuclease from mothers, fathers or both are denoted by superscripts 10, 01 or 11.

e_{W}=(F_{WW}+w_{WW}¹⁰F_{WW}¹⁰+w_{WW}⁰¹F_{WW}⁰¹+w_{WW}^{1:}F_{WW}^{1:}+(F_{WR}+w_{WR}¹⁰F_{WR}¹⁰+w_{WR}⁰¹F_{WR}⁰¹+w_{WR}¹¹F_{WR}¹¹)/2)w̅_{f}

e_{R}=½(F_{WR}+w_{WR}¹⁰F_{WR}¹⁰+w_{WR}⁰¹F_{WR}⁰¹+w_{WR}¹¹F_{WR}¹¹)/w̅_{f}

e_{W}*=(1−d_{f})(1−u_{f})(w_{WD}¹⁰F_{WD}¹⁰+w_{WD}⁰¹F_{WD}⁰¹+w_{WD}¹¹F_{WD}¹¹)/w̅_{f}

e_{D}*=d_{f}(w_{WD}¹⁰F_{WD}¹⁰+w_{WD}⁰¹F_{WD}⁰¹+w_{WD}¹¹F_{WD}¹¹)/w̅_{f}

e_{R}*=(1−d_{f})u_{f}(w_{WD}¹⁰F_{WD}¹⁰+w_{WD}⁰¹F_{WD}⁰¹+w_{WD}¹¹F_{WD}¹¹)/w̅_{f}

The proportions sᵢ of type i alleles in sperm are:

s_{W}=(M_{WW}+M_{WW}¹⁰+M_{WW}⁰¹+M_{WW}¹¹+(M_{WR}+M_{WR}¹⁰+M_{WR}⁰¹+M_{WR}¹¹)/2)/w̅ₘ

s_{R}=(M_{RR}+(M_{WR}+M_{WR}¹⁰+M_{WR}⁰¹+M_{WR}¹¹)/2)/w̅ₘ

s_{W}*=(1−dₘ)(1−uₘ)(M_{WD}¹⁰+M_{WD}⁰¹+M_{WD}¹¹)/w̅ₘ

s_{D}*=(M_{DD}+M_{DR}/2+dₘ(M_{WD}¹⁰+M_{WD}⁰¹+M_{WD}¹¹))/w̅ₘ

s_{R}*=(M_{DR}/2+(1−dₘ)uₘ(M_{WD}⁰¹+M_{WD}¹⁰+M_{WD}¹¹))/w̅ₘ

Above, w̅_{f} and w̅ₘ are the average female and male fitness:

w̅_{f}=F_{WW}+w_{WW}¹⁰F_{WW}¹⁰+w_{WW}⁰¹F_{WW}⁰¹+w_{WW}¹¹F_{WW}¹¹+w_{WD}¹⁰F_{WD}¹⁰+w_{WD}⁰¹F_{WD}⁰¹+w_{WD}¹¹F_{WD}¹¹+F_{WR}+F_{WR}¹⁰w_{WR}¹⁰+w_{WR}⁰¹F_{WR}⁰¹+w_{WR}¹¹F_{WR}¹¹

w̅ₘ=M_{WW}+M_{WW}¹⁰+M_{WW}³¹+M_{WW}¹¹+M_{WD}¹⁰+M_{WD}⁰¹+M_{WD}¹¹+M_{WR}+M_{WR}¹⁰+M_{WR}⁰¹+M_{WR}¹¹+M_{DD}+M_{DR}+M_{RE}=1

To model cage experiments, the inventors started with an equal number of males and females, with an initial frequency of wildtype females in the female population of F_WW=1, wildtype males in the male population of M_{WW}=½, and M_{WD}⁰¹=½ heterozygote drive males that inherited the drive from their fathers. Assuming a 50:50 ratio of males and females in progeny, after the starting generation, genotype frequencies of type i/j in the next generation (t+1) are the same in males and females, Fᵢⱼ (t+1)=Mᵢⱼ (t+1). Both are given by Gᵢⱼ (t+1) in the following set of equations in terms of the gamete proportions in the previous generation, assuming random mating:

G_{WW}(t+1)=e_{W}s_{W}

G_{WW}¹⁰(t+1)=e_{W}*s_{W}

G_{WW}⁰¹(t+1)=e_{W}s_{W}*

G_{WW}¹¹(t+1)=e_{W}*s_{W}*

G_{WD}¹⁰(t+1)=e_{D}*s_{W}

G_{WD}⁰¹(t+1)=e_{W}s_{D}*

G_{WD}¹¹(t+1)=e_{W}*s_{D}*+e_{D}*s_{W}*

G_{WR}(t+1)=e_{W}s_{R}+e_{R}s_{W}

G_{WR}¹⁰(t+1)=e_{W}*s_{R}+e_{R}*s_{W}

G_{WR}⁰¹(t+1)=e_{W}s_{R}*+e_{R}s_{W}*

G_{WR}¹¹(t+1)=e_{W}*s_{R}*+e_{R}*s_{W}*

G_{DD}(t+1)=eₙ*sₙ*

G_{DR}(t+1)=(e_{R}+e_{R}*)s_{D}*+e_{D}*(s_{R}+s_{R}*)

G_{RR}=(e_{R}+e_{R}*)(s_{R}+s_{R}*)

The frequency of transgenic individuals can be compared with experiment (fraction of RFP+ individuals):

f_{RFP}+=F_{WD}¹⁰+F_{WD}⁰¹+F_{WD}¹¹+F_{DD}+F_{DR}+M_{WD}¹⁰+M_{WD}⁰¹+M_{WD}¹¹+M_{DD}+M_{DR}

All calculations were carried out using Wolfram Mathematica²³.

### PCR

The PCR reactions were performed using Phusion High Fidelity Master Mix. Initial denaturation was performed in 98° C. for 30 seconds. Primer annealing was performed at a temperature range of 60-72° C. form 30 seconds and elongation was performed at a temperature of 72° C. for 30 seconds per kb.

TABLE 2
Primers used in this study for Example 1

| | |
|---|---|
| dsxgRNA-F | TGCTGTTTAACACAGGTCAAGCGG-SEQ ID No: 14 |
| dsxgRNA-R | AAACCCGCTTGACCTGTGTTAAAC-SEQ ID No: 15 |
| dsx031L-F | GCTCGAATTAACCATTGTGGACCGGTCTTGTGTTTAGCAG |
| | GCAGGGGA-SEQ ID No: 16 |
| dsx031L-R | TCCACCTCACCCATGGGACCCACGCGTGGTGCGGGTCACC |
| | GAGATGTTC-SEQ ID No: 17 |
| dsx031R-F | CACCAAGACAGTTAACGTATCCGTTACCTTGACCTGTGTTA |
| | AACATAAAT-SEQ ID No: 18 |
| dsx031R-R | GGTGGTAGTGCCACACAGAGAGCTTCGCGGTGGTCAACG |
| | AATACTCACG-SEQ ID No: 19 |
| zpgprCRISPR-F | GCTCGAATTAACCATTGTGGACCGGTCAGCGCTGGCGGTG |
| | GGGA-SEQ ID No: 20 |
| zpgprCRISPR-R | TCGTGGTCCTTATAGTCCATCTCGAGCTCGATGCTGTATTT |
| | GTTGT-SEQ ID No: 21 |
| zpgteCRISPR-F | AGGCAAAAAAGAAAAAGTAATTAATTAAGAGGACGGCGA |
| | GAAGTAATCAT-SEQ ID No: 22 |
| zpgteCRISPR-R | TTCAAGCGCACGCATACAAAGGCGCGCCTCGCATAATGAA |
| | CGAACCAAAGG-SEQ ID No: 23 |
| dsxin3-F | GGCCCTTCAACCCGAAGAAT-SEQ ID No: 24 |
| dsxex6-R | CTTTTTGTACAGCGGTACAC-SEQ ID No: 25 |
| GFP-F | GCCCTGAGCAAAGACCCCAA-SEQ ID No: 26 |
| dsxex4-F | GCACACCAGCGGATCGACGAAG-SEQ ID No: 27 |
| dsxex5-R | CCCACATACAAAGATACGGACAG-SEQ ID No: 28 |
| dsxex6-R | GAATTTGGTGTCAAGGTTCAGG-SEQ ID No: 29 |
| 3xP3 | TATACTCCGGCGGTCGAGGGTT-SEQ ID No: 30 |
| hCas9-F | CCAAGAGAGTGATCCTGGCCGA-SEQ ID No: 31 |
| dsxex5-R1 | CTTATCGGCATCAGTTGCGCAC-SEQ ID No: 32 |
| dsxin4-F | GGTGTTATGCCACGTTCACTGA-SEQ ID No: 33 |
| RFP-R | CAAGTGGGAGCGCGTGATGAAC-SEQ ID No: 34 |

TABLE 6
Primers used in this study for Example 2

| | |
|---|---|
| multidsxΦ31L-F | gctcgaattaaccattgtggaccggtCTTGTGTTTAGCAGGCAGGGGA-SEQ |
| | ID No: 52 |
| multidsxΦ31L-R | tgaacgattggggtaccggtCTTGACCTGTGTTAAACATAAATG-SEQ ID |
| | No: 53 |
| multidsxΦ31R-F | agatataatcctgaacgcgtGAGTGGATGATAAACTTTCCGCAC-SEQ ID |
| | No: 54 |
| multidsxΦ31R-R | tccacctcacccatgggacccacgcgtGGTGCGGGTCACCGAGATGTTC- SEQ |
| | ID No: 55 |
| 4050-2U6-T1-F | gagggtctcaTGCTGTTTAACACAGGTCAAGCGGgttttagagctagaaatagca |
| | agt-SEQ ID No: 56 |
| 4050-2U6-T3-R | gagggtctcaAAACCTCTGACGGGTGGTATTGCagcagagagcaactccatttca |
| | t-SEQ ID No: 57 |

### Example 1

To investigate whether dsx represented a suitable target for a gene drive approach aimed at suppressing population reproductive capacity, the inventors disrupted the intron 4-exon 5 boundary of dsx with the objective to prevent the formation of functional AgdsxF while leaving the AgdsxM transcript unaffected. The inventors injected A. gambiae embryos with a source of Cas9 and gRNA designed to selectively cleave the intron 4-exon 5 boundary in combination with a template for homology directed repair (HDR) to insert an eGFP transcription unit (FIG. 1c). Transformed individuals were intercrossed to generate homozygous and heterozygous mutants among the progeny.

### Results

HDR-mediated integration was confirmed by diagnostic PCR using primers that spanned the insertion site, producing a larger amplicon of the expected size for the HDR event and a smaller amplicon for the wild type allele, and thus allowing easy confirmation of genotypes (FIG. 1d).

The knock-in of the eGFP construct resulted in the complete disruption of the exon 5 (dsxF−) coding sequence and was confirmed by PCR and genomic sequencing of the chromosomal integration (FIG. 6 and data not shown). Crosses of heterozygote individuals produced, wild type, heterozygous and homozygous individuals for the dsxF− allele at the expected Mendelian ratio 1:2:1, indicating that there was no obvious lethality associated with the mutation during development (Table 3).

TABLE 3
Ratio of larvae recovered by intercrossing
heterozygous dsx ΦC31-knock-in mosquitoes

| GFP strong (dsxF^{-/-}) | GFP weak (dsxF^{-/+}) | no GFP (+/+) | Total |
|---|---|---|---|
| 262 (24.9%) | 523 (49.7%) | 268 (25.5%) | 1053 |

Larvae heterozygous for the exon 5 disruption developed into adult male and female mosquitoes with a sex ratio close to 1:1. On the contrary half of dsxF−/− individuals developed into normal males whereas the other half showed the presence of both male and female morphological features as well as a number of developmental anomalies in the internal and external reproductive organs (intersex).

To establish the sex genotype of these dsxF^{-/-} intersex, the inventors introgressed the mutation into a line containing a Y-linked visible marker (RFP) and used the presence of this marker to unambiguously assign sex genotype among individuals heterozygous and homozygous for the null mutation. This approach revealed that the intersex phenotype was observed only in genotypic females that were homozygous for the null mutation. The inventors saw no effect in heterozygous mutants, suggesting that the female-specific isoform of dsx is haplosufficient.

Examination of external sexually dimorphic structures in dsxF^{-/-} genotypic females showed several phenotypic abnormalities including: the development of dorsally rotated male claspers (and absent female cerci), longer flagellomeres associated with male-like plumose antennae (FIG. 2). The analysis of the internal reproductive organs of these individuals failed to reveal the presence of fully developed ovaries and spermathecae; instead they were replaced by male-accessory glands (MAGs) and in some cases (˜20%) by rudimentary pear-shaped organs resembling unstructured testes (FIG. 7).

Males carrying the dsxF− null mutation in heterozygosity or homozygosity showed wild type levels of fertility as measured by clutch size and larval hatching per mated female, as did heterozygous dsxF− female mosquitoes. On the contrary, intersex XX dsxF-female mosquitoes, though attracted to anaesthetised mice were unable to take a bloodmeal and failed to produce any eggs (FIG. 3).

The surprisingly drastic phenotype of dsxF− in females is proof of key functional role of exon 5 of dsx in the poorly understood sex differentiation pathway of A. gambiae mosquitoes and suggested that its sequence could represent a suitable target for gene drive approaches aimed at population suppression.

The inventors employed recombinase-mediated cassette exchange (RMCE) to replace the 3×P3::GFP transcription unit with a dsxF^{CRISPRh} gene drive construct that consists of an RFP marker gene, a transcription unit to express the gRNA targeting dsxF, and the Cas9 gene under the control of the germline promoter of zero population growth (zpg) and its terminator sequence (FIG. 8). The zpg promoter has shown improved germline restriction of expression and specificity over the vasa promoter used in previous gene drive constructs (Hammond and Crisanti unpublished). Successful RMCE events that incorporated the dsxF^{CRISPRh} into its target locus were confirmed in those individuals that had swapped the GFP for the RFP marker. During meiosis the Cas9/gRNA complex cleaves the wild type allele at the target sequence and the dsxF^{CRISPRh} cassette is copied into wt locus via HDR (‘homing’), disrupting exon 5 in the process.

The ability of the dsxF^{CRISPRh} construct to home and bypass Mendelian inheritance was analysed by scoring the rates of RFP inheritance in the progeny of heterozygous parents (referred to as dsxF^{CRISPRh}/+ hereafter) crossed to wild type mosquitoes. Surprisingly, high dsxF^{CRISPRh} transmission rates of up to 100% were observed in the progeny of both heterozygous dsxF^{CRISPRh}/+ male and female mosquitoes (FIG. 4a). The fertility of the dsxF^{CRISPRh} line was also assessed to unravel potential negative effects due to ectopic expression of the nuclease in somatic cells and/or parental deposition of the nuclease into the newly fertilised embryos (FIG. 4b). These experiments showed that while heterozygous dsxF^{CRISPRh}/+ males showed a fecundity rate (assessed as larval progeny per fertilised female) that did not differ from wild type males, heterozygous dsxF^{CRISPRh}/+ female showed reduced fecundity overall (mean fecundity 49.8%+/−6.3% S.E., p<0.0001).

Surprisingly, the inventors noticed a more severe reduction in the fertility of heterozygous females when the drive allele was inherited from their father (mean fecundity 21.7%+/−8.6%) rather than their mother (64.9%+/−6.9%) (FIG. 9). Without wishing to be bound to any particular theory, the inventors believe that this could be explained assuming a paternal deposition of active Cas9 nuclease into the newly fertilized zygote that stochastically induces conversion to of dsx to dsxF⁻, either through end-joining or HDR, in a significant number of cells resulting in a reduced fertility in females. Consistent with this hypothesis, some heterozygous females receiving a paternal dsxF^{CRISPRh} allele showed a somatic mosaic phenotype that included, with varying penetrance, the absence of spermatheca and/or the formation of an incomplete clasper set. A mathematical model built considering the inheritance bias of the construct, the fecundity of heterozygous individuals, the phenotype of intersex as well as the paternal deposition of the nuclease on female fertility, indicated that the dsxF^{CRISPRh} had the potential to reach 100% frequency in caged population in a span of 9-13 generations depending on starting frequency and stochasticity (FIG. 5a).

To test this hypothesis, caged wild type mosquito populations were mixed with individuals carrying the dsxF^{CRISPRh} allele and subsequently monitored at each generation to assess the spread of the drive and quantify its effect on reproductive output. To mimic a hypothetical release scenario, the inventors started the experiment in two replicate cages putting together 300 wild type female mosquitoes with 150 wt male mosquitoes and 150 dsxF^{CRISPRh}/+ male individuals and allowed them to mate. Eggs produced from the whole cage were counted and 650 eggs were randomly selected to seed the next generations. The larvae that hatched from the eggs were screened for the presence of the RFP marker to score the number of the progeny containing the dsxF^{CRISPRh} allele in each generation. During the first three generations, the inventors observed in both caged populations an increase of the drive allele from 25% up to ˜69% and thereafter they diverged. In cage 2 the drive reached 100% frequency by generation 7; in the following generation no eggs were produced and the population collapsed. In cage 1 the drive allele reached 100% frequency at generation 11 after drifting around 65% for two generations. This cage population also failed to produce eggs in the next generation. Though the two cages showed some apparent differences in the dynamics of spreading both curves fall within the prediction of the model (FIG. 5b). A summary of the cage trials is shown in table 5.

The inventors also monitored at different generations the occurrence of mutations at the target site to identify the occurrence of nuclease resistant functional variants. Amplicon sequencing of the target sequence from pooled population samples collected at generation 2, 3, 4 and 5 revealed the presence of several low frequency indels generated at the cleavage site, none of which appeared to encode for a functional AgdsxF transcript (FIGS. 10A-C). Accordingly, none of the variants identified showed any signs of positive selection as the drive progressively increased in frequency over generations, thus indicating that the selected target sequence has rigid functional and structural constraints. This notion is supported by the high degree of conservation of exon 5 in A. gambiae mosquitoes^{16,17} and the presence of highly regulated splice site critical for the mosquito reproductive biology.

Heterozygous and homozygous individuals for the dsxF allele were separated based on the intensity of fluorescence afforded by the GFP transcription unit within the knockout allele. Homozygous mutants were distinguishable as recovered in the expected Mendelian ratio of 1:2:1 suggesting that the disruption of the female-specific isoform of Agdsx is not lethal at the Li larval stage.

TABLE 4
Genetic females homozygous for the insertion
carry male-specific characteristics

| | Genetic Males | | | Genetic Females | | |
|---|---|---|---|---|---|---|
| Characteristic | dsxF^{+/+} | dsxF^{+/-} | dsxF^{-/-} | dsxF^{+/+} | dsxF^{+/-} | dsxF^{-/-} |
| Pupal genital | male | male | male | female | female | male |
| lobe | | | | | | |
| Claspers | ✓ | ✓ | ✓ | X | X | ✓ |
| Cercus | X | X | X | ✓ | ✓ | X |
| Spermatheca | X | X | X | ✓ | ✓ | X |
| MAGs | ✓ | ✓ | ✓ | X | X | ✓ |
| Feed on blood | X | X | X | ✓ | ✓ | X |
| Can lay eggs | X | X | X | ✓ | ✓ | X |
| Plumose | ✓ | ✓ | ✓ | X | X | ✓ |
| antennae | | | | | | |
| Pilose | X | X | X | ✓ | ✓ | X |
| antennae | | | | | | |

The inventors assume that parental effects on fitness (egg production and hatching rates) for non-drive (W/W, W/R) females with nuclease from one or both parents are the same as observed values for drive heterozygote (W/D) females with parental effects. For combined maternal and paternal effects (nuclease from both parents), the minimum of the observed values for maternal and paternal effect is assumed.

TABLE 5
Summary of values obtained from the cage trials

| | Cage Trial 1 | | | | Cage Trial 2 | | | |
|---|---|---|---|---|---|---|---|---|
| Genera- | Transgenic | Hatching | Egg Output | Repr. | Transgenic | Hatching | Egg Output | Repr. |
| tion | Rate (%) | Rate (%) | (N) | Load (%) | Rate (%) | Rate (%) | (N) | Load (%) |
| G0 | 25 | — | 27462 | — | 25 | — | 26895 | — |
| | (150/600) | | | | (150/600) | | | |
| G1 | 49.65 | 88.62 | 17405 | 36.62 | 50 | 86.15 | 16578 | 38.36 |
| | (268/576) | (576/650) | | | (280/560) | (560/650) | | |
| G2 | 62.01 | 74.92 | 14957 | 45.54 | 61.79 | 80.92 | 15565 | 42.13 |
| | (302/487) | (487/650) | | | (325/526) | (526/650) | | |
| G3 | 68.94 | 76.77 | 11249 | 59.04 | 68.05 | 74.15 | 9376 | 65.14 |
| | (344/499) | (499/650) | | | (328/482) | (482/650) | | |
| G4 | 67.67 | 71.85 | 9170 | 66.61 | 85.41 | 71.69 | 6514 | 75.78 |
| | (316/467) | (467/650) | | | (398/466) | (466/650) | | |
| G5 | 58.67 | 69.23 | 11364 | 58.62 | 86.5 | 61.54 | 4805 | 81.13 |
| | (264/450) | (450/650) | | | (346/400) | (400/650) | | |
| G6 | 63.3 | 70 | 7727 | 71.86 | 90.09 | 52.77 | 4210 | 84.35 |
| | (288/455) | (455/650) | | | (309/343) | (343/650) | | |
| G7 | 69.47 | 78.62 | 7785 | 71.65 | 100 | 55.85 | 1668 | 93.8 |
| | (355/511) | (511/650) | | | (363/363) | (363/650) | | |
| G8 | 70.07 | 70.92 | 6293 | 77.08 | 100 | 42.77 | 0 | 100 |
| | (323/461) | (461/650) | | | (278/278) | (278/650) | | |
| G9 | 75.58 | 66.15 | 4107 | 85.04 | — | — | — | — |
| | (325/430) | (430/650) | | | | | | |
| G10 | 95.71 | 57.38 | 4146 | 84.90 | | | | |
| | (357/373) | (373/650) | | | | | | |
| G11 | 100 | 57.54 | 2645 | 90.37 | | | | |
| | (374/374) | (374/650) | | | | | | |
| G12 | 100 | 38.92 | 0 | 100 | | | | |
| | (253/253) | (253/650) | | | | | | |

Transgenic rate, hatching rate, egg output and reproductive load at each generation during the cage experiment. The reproductive load indicates the suppression of egg production at each generation compared to the first generation.

### CONCLUSIONS

In the human malaria vector, Anopheles gambiae, the gene doublesex (Agdsx) encodes two alternatively spliced transcripts dsx-female (AgdsxF) and dsx-male (AgdsxM) that, in turn, regulate the activation of distinct subordinate genes responsible for the differentiation of the two sexes. The female transcript, unlike AgdsxM, contains an exon (exon 5) whose coding sequence is highly conserved in all Anopheles mosquitoes so far analysed. CRISPR-Cas9 targeted disruption of the intron 4-exon 5 sequence boundary aimed at blocking the formation of functional AgdsxF did not affect male development or fertility, whereas females homozygous for the disrupted allele showed an intersex phenotype characterised by the presence of male internal and external reproductive organs and complete sterility, as summarised in table 4. A CRISPR-Cas9 gene drive construct targeting this same sequence was able to spread rapidly in caged mosquito populations reaching 100% prevalence within a span of 8-12 generations while progressively reducing the egg production to the point of total population collapse. Notably, this drive solution did not induce resistance. A variety of non-functional Cas9 resistant variants were generated in each generation at the target site, they all failed to block the spread of the drive.

Hence, these data all together provide important functional insights on the role of dsx in A. gambiae sex determination while demonstrating substantial progress towards the development of effective gene drive vector control measures aimed at population suppression. Without wishing to be bound to any particular theory, the intersex phenotype of dsxF−/− genetic females demonstrates that exon 5 is critical for the production of a functional female transcript. Furthermore, the observation that heterozygous dsxFCRISPRh/+ females are fertile and produce nearly 100% transformed progeny would indicate that the majority of the germ cells in these females are homozygous and, unlike somatic cells, do not undergo autonomous dsx-mediated sex commitment¹⁸. The development of a gene drive solutions capable of collapsing a human malaria vector population is a long sought scientific and technical achievement¹⁹. The gene drive dsxFCRISPRh targeting exon 5 of dsx showed a number of desired efficacy features for field applications, in term of inheritance bias, fertility of heterozygous individuals, phenotype of homozygous females and apparent lack of nuclease-resistant functional variants at the target site.

### Example 2

A promising approach to mitigate resistance to gene drive is to target multiple sites at the same time in a strategy analogous to combinational drug therapy. For resistance to get selected against the gene drive, resistant mutations would have to be simultaneously present at all target sites, and co-operatively restore the targeted gene's original function. Note that homing will also serve to remove resistant mutations generated if at least one of the targeted sites is still cleavable.

Exon 5 of doublesex that was targeted with a gene drive as described in Example 1 contains a total of four invariant target sites that are amenable to multiplexing (FIG. 12). Accordingly, the inventors then generated a novel multiplexed gene drive targeting the original target site at doublesex (T1) and a new target site (T3) present at the 3′ end of the exon 5 coding sequence. The transgenic line that was obtained contains a CRISPR construct bearing a 3×P3::RFP marker, Cas9 expressed under the zpg promoter and two multiplexed U6::gRNA expression cassettes as shown in FIG. 13.

The inheritance bias of the gene drive, and fertility of gene drive carriers was assessed through phenotype assays. Gene drive heterozygotes of both sexes that had inherited the drive from either males or females were crossed to wild-type individuals and females of each cross were allowed to lay eggs individually. The same was done with a wild-type cage, as a control. Egg and larval output of each female was counted, as soon as they laid and hatched respectively. Larvae were then screened for RFP fluorescence indicative of gene drive presence. The mating status of females that did not give offspring was determined by dissecting their spermathecae and examining it under an EVOS cell imaging microscope for the presence of spermatozoa. Females that showed no evidence of mating were all included in the analysis as having given 0 progeny, since mating competence can be affected by carrying the doublesex gene drive. The results from Kyrou et al. (2018) were adapted to also include unmated individuals in the analysis.

The results revealed that the novel multiplexed gene drive can successfully bias its inheritance to the next generation with transmission rates comparable to the single-guide gene drive we previously developed (p>0.05) or higher (p=0.04), when the gene drive was transmitted by a male carrier who inherited it maternally (F->M class) (FIGS. 14A and 14B). As with the original doublesex gene drive, the fertility of gene drive carrier females descended from transgenic males (M->F class) was decreased compared to all other classes (FIGS. 14C and 14D). The total and relative number of average larval progeny of females that inherited the gene drive from males (M->F class), is surprisingly higher for the multiplexed gene drive (FIGS. 14C and 14D).

### REFERENCES

1. Gantz, V. M. et al. Highly efficient Cas9-mediated gene drive for population modification of the malaria vector mosquito Anopheles stephensi. Proc Natl Acad Sci USA 112, E6736-6743 (2015).
2. Hammond, A. et al. A CRISPR-Cas9 gene drive system targeting female reproduction in the malaria mosquito vector Anopheles gambiae. Nat Biotechnol 34, 78-83 (2016).
3. Burt, A. Site-specific selfish genes as tools for the control and genetic engineering of natural populations. Proc Biol Sci 270, 921-928 (2003).
4. Deredec, A., Godfray, H. C. & Burt, A. Requirements for effective malaria control with homing endonuclease genes. Proc Natl Acad Sci USA 108, E874-880 (2011).
5. Hamilton, W. D. Extraordinary sex ratios. A sex-ratio theory for sex linkage and inbreeding has new implications in cytogenetics and entomology. Science 156, 477-488 (1967).
6. Galizi, R. et al. A synthetic sex ratio distortion system for the control of the human malaria mosquito. Nat Commun 5, 3977 (2014).
7. Magnusson, K. et al. Demasculinization of the Anopheles gambiae X chromosome. BMC Evol Biol 12, 69 (2012).
8. Champer, J. et al. Novel CRISPR/Cas9 gene drive constructs reveal insights into mechanisms of resistance allele formation and drive efficiency in genetically diverse populations. PLoS Genet 13, e1006796 (2017).
9. Hammond, A. M. et al. The creation and selection of mutations resistant to a gene drive over multiple generations in the malaria mosquito. PLoS Genet 13, e1007039 (2017).
10. Marshall, J. M., Buchman, A., Sanchez, C. H. & Akbari, O. S. Overcoming evolved resistance to population-suppressing homing-based gene drives. Sci Rep 7, 3776 (2017).
11. Unckless, R. L., Clark, A. G. & Messer, P. W. Evolution of Resistance Against CRISPR/Cas9 Gene Drive. Genetics 205, 827-841 (2017).
12. Burtis, K. C. & Baker, B. S. Drosophila doublesex gene controls somatic sexual differentiation by producing alternatively spliced mRNAs encoding related sex-specific polypeptides. Cell 56, 997-1010 (1989).
13. Graham, P., Penn, J. K. & Schedl, P. Masters change, slaves remain. Bioessays 25, 1-4 (2003).
14. Krzywinska, E., Dennison, N.J., Lycett, G. J. & Krzywinski, J. A maleness gene in the malaria mosquito Anopheles gambiae. Science 353, 67-69 (2016).
15. Scali, C., Catteruccia, F., Li, Q. & Crisanti, A. Identification of sex-specific transcripts of the Anopheles gambiae doublesex gene. J Exp Biol 208, 3701-3709 (2005).
16. Neafsey, D. E. et al. Mosquito genomics. Highly evolvable malaria vectors: the genomes of 16 Anopheles mosquitoes. Science 347, 1258522 (2015).
17. Anopheles gambiae Genomes, C. et al. Genetic diversity of the African malaria vector Anopheles gambiae. Nature 552, 96-100 (2017).
18. Murray, S. M., Yang, S. Y. & Van Doren, M. Germ cell sex determination: a collaboration between soma and germline. Curr Opin Cell Biol 22, 722-729 (2010).
19. Curtis, C. F. Possible use of translocations to fix desirable genes in insect pest populations. Nature 218, 368-369 (1968).
20. National Academies of Sciences, E. & Medicine Gene Drives on the Horizon: Advancing Science, Navigating Uncertainty, and Aligning Research with Public Values. (The National Academies Press, Washington, D.C.; 2016).
21. Papathanos, P. A., Windbichler, N., Menichelli, M., Burt, A. and Crisanti, A. The vasa regulatory region mediates germline expression and maternal transmission of proteins in the malaria mosquito Anopheles gambiae: a versatile tool for genetic control strategies. BMC Mol Biol 10, 65, (2009).
22 Hammond, A. M. et al. The creation and selection of mutations resistant to a gene drive over multiple generations in the malaria mosquito. PLoS Genet 13, e1007039 (2017).
23. Wolfram Research, Inc., 2017 Mathematica 11.2, Champaign, Ill.

1. A gene drive genetic construct capable of disrupting an intron-exon boundary of the female-specific splice form of the doublesex gene in an arthropod, such that when the construct is expressed, the intron-exon boundary is disrupted and at least one exon is spliced out of a doublesex precursor-mRNA transcript, wherein a female arthropod, which is homozygous for the construct, exhibits a suppressed reproductive capacity.

2. The gene drive genetic construct according to claim 1, wherein the arthropod is an insect,
optionally wherein the insect is a mosquito,
optionally, wherein the mosquito is of the subfamily Anophelinae, and
optionally wherein the mosquito is selected from a group consisting of: Anopheles gambiae; Anopheles coluzzi; Anopheles merus; Anopheles arabiensis; Anopheles quadriannulatus; Anopheles stephensi; Anopheles funestus; and Anopheles melas.

3. (canceled)

4. The gene drive genetic construct according to claim 1, wherein the arthropod is Anopheles gambiae.

5. The gene drive genetic construct according to claim 1, wherein the doublesex gene comprises a nucleic acid sequence substantially as set out in SEQ ID NO: 1, or a fragment or variant thereof.

6. The gene drive genetic construct according to claim 1, wherein the intron-exon boundary targeted by the genetic construct is the boundary between intron 4 and exon 5 of the doublesex gene, optionally wherein the genetic construct targets a nucleic acid sequence comprising or consisting of the nucleotide sequence substantially as set out in SEQ ID NO: 2, 3 or 4, or a fragment or variant thereof, or wherein the target sequence includes up to 1, 2, 3, 4, 5, 10 or 15 nucleotides 5′ and/or 3′ of SEQ ID No:2, 3 or 4.

7. The gene drive genetic construct according to claim 1, wherein the gene drive genetic construct is a nuclease-based genetic construct,
optionally wherein the nuclease-based genetic construct is selected from a group consisting of: a transcription activator-like effector nuclease (TALEN) genetic construct; Zinc finger nuclease (ZFN) genetic construct; and a CRISPR-based gene drive genetic construct.

8. (canceled)

9. The gene drive genetic construct according to claim 1, wherein the gene drive genetic construct is a nuclease-based genetic construct and wherein the gene drive genetic construct is a CRISPR-based gene drive construct, optionally wherein the genetic construct is a CRISPR-Cpf1-based or a CRISPR-Cas9-based gene drive genetic construct.

10. The gene drive genetic construct according to claim 1, wherein the construct is a nuclease-based genetic construct and is selected from a group consisting of: a transcription activator-like effector nuclease (TALEN) genetic construct; Zinc finger nuclease (ZFN) genetic construct; and a CRISPR-based gene drive genetic construct, wherein the genetic construct comprises a first nucleotide sequence encoding a nucleotide sequence that is capable of hybridising to the intron-exon boundary of the doublesex gene,
optionally wherein the first nucleotide sequence that is capable of hybridising to the intron-exon boundary of the doublesex gene is a guide RNA,
optionally, wherein the first nucleotide sequence encoding a nucleotide sequence that is capable of hybridising to the intron-exon boundary of the doublesex (dsx) gene comprises a nucleic acid sequence substantially as set out in SEQ ID NO: 5 or 6, or a fragment or variant thereof and
optionally, wherein the nucleotide sequence which is encoded by the first nucleotide sequence and which is capable of hybridising to the intron-exon boundary of the doublesex (dsx) gene comprises a nucleic acid sequence substantially as set out in SEQ ID NO: 58 or 48, or a fragment or variant thereof.

11. (canceled)

12. (canceled)

13. The gene drive genetic construct according to claim 1, wherein the construct is a nuclease-based genetic construct and is selected from a group consisting of: a transcription activator-like effector nuclease (TALEN) genetic construct; Zinc finger nuclease (ZFN) genetic construct; and a CRISPR-based gene drive genetic construct, and wherein the gene drive genetic construct further comprises a second nucleotide sequence encoding a CRISPR nuclease, optionally wherein the second nucleotide sequence encodes a Cpf1 or Cas9 nuclease.

14. The gene drive genetic construct according to claim 1, wherein the construct is a nuclease-based genetic construct and is selected from a group consisting of: a transcription activator-like effector nuclease (TALEN) genetic construct; Zinc finger nuclease (ZFN) genetic construct; and a CRISPR-based gene drive genetic construct, and wherein the gene drive genetic construct further comprises at least one promoter sequence, which drives expression of the first and second nucleotide sequence, optionally wherein the gene drive genetic construct comprises a first promoter sequence operably linked to the first nucleotide sequence and a second promoter sequence operably linked to the second nucleotide sequence.

15. (canceled)

16. (canceled)

17. The gene drive genetic construct according to claim 1,
wherein the construct is a nuclease-based genetic construct and is selected from a group consisting of: a transcription activator-like effector nuclease (TALEN) genetic construct; Zinc finger nuclease (ZFN) genetic construct; and a CRISPR-based gene drive genetic construct, and wherein the gene drive genetic construct further comprises at least one promoter sequence, which drives expression of the first and second nucleotide sequence, wherein the gene drive genetic construct comprises a first promoter sequence operably linked to the first nucleotide sequence and a second promoter sequence operably linked to the second nucleotide sequence and
wherein the second promoter sequence is a promoter sequence that substantially restricts expression of the second nucleotide sequence to germline cells of the arthropod, optionally wherein the second promoter sequence is:
(i) zpg, optionally wherein the second promoter sequence comprises or consists of a nucleic acid sequence substantially as set out in SEQ ID No: 7, or a variant or fragment thereof;
(ii) nos, optionally wherein the second promoter sequence comprises or consists of a nucleic acid sequence substantially as set out in SEQ ID No: 8, or a variant or fragment thereof;
(iii) exu, optionally wherein the second promoter sequence comprises or consists of a nucleic acid sequence substantially as set out in SEQ ID No: 9, or a variant or fragment thereof; or
(iv) vasa2, optionally wherein the second promoter sequence comprises or consists of a nucleic acid sequence substantially as set out in SEQ ID No: 10, or a variant or fragment thereof.

18. (canceled)

19. (canceled)

20. The gene drive genetic construct according to claim 1, wherein the third nucleotide sequence comprises or consists of a nucleic acid sequence substantially as set out in SEQ ID No: 11, or a variant or fragment thereof and/or wherein the fourth nucleotide sequence comprises or consists of a nucleic acid sequence substantially as set out in SEQ ID No: 12, or a variant or fragment thereof.

21. The gene drive genetic construct according to claim 1, wherein the gene drive construct comprises or consists of a nucleic acid sequence substantially as set out in SEQ ID NO: 13, or a fragment or variant thereof.

22. The gene drive genetic construct according to claim 1, wherein the construct is capable of targeting (i) a first target site which comprises the intron-exon boundary of the female specific splice form of the doublesex (dsx) gene, and (ii) a second target site disposed in exon 5 of the female specific splice form of the doublesex (dsx) gene, optionally wherein
(i) the second target site comprises or consists of a nucleic acid sequence, which is disposed in the sequence substantially as set out in SEQ ID No: 35, 36 (T2), 37 (T3) or 38 (T4) or a variant or fragment thereof, or wherein the second target site includes up to 1, 2, 3, 4, 5, 10 or 15 nucleotides 5′ and/or 3′ of SEQ ID No:35, 36, 37 or 38; or
(ii) the second target site comprises or consists of a nucleic acid sequence, which is disposed in the sequence substantially as set out in SEQ ID No: 35, 36 (T2), 37 (T3) or 38 (T4) or a variant or fragment thereof, or wherein the second target site includes up to 1, 2, 3, 4, 5, 10 or 15 nucleotides 5′ and/or 3′ of SEQ ID No:35, 36, 37 or 38.

23-34. (canceled)

35. A use of a gene drive genetic construct to disrupt an intron-exon boundary of the female specific splice form of the doublesex gene in an arthropod, such that when the construct is expressed, the exon is spliced out of a doublesex precursor-mRNA transcript, wherein the female arthropod's reproductive capacity is suppressed when females are homozygous for the construct.

36. A method for preventing or reducing the inclusion of at least one exon into the female specific splice form of arthropod doublesex mRNA, when said mRNA is produced by splicing from a precursor mRNA transcript, the method comprising contacting one or more cells of an arthropod, optionally one or more cells of an arthropod embryo, in vitro or ex vivo, under conditions conducive to uptake of a gene drive genetic construct that capable of disrupting an intron-exon boundary of the female-specific splice form of the doublesex gene in an arthropod, such that when the construct is expressed, the intron-exon boundary is disrupted and at least one exon is spliced out of a doublesex precursor-mRNA transcript, wherein a female arthropod, which is homozygous for the construct, exhibits a suppressed reproductive capacity by such cell, and allowing splicing to take place, or
a method of producing a genetically modified arthropod, the method comprising introducing into an arthropod a gene drive genetic construct capable of disrupting an intron/exon boundary of the female specific splice form of doublesex gene in an arthropod, such that when the gene-drive construct is expressed, an exon is spliced out of a doublesex precursor-mRNA transcript, wherein a female arthropod, which is homozygous for the construct, exhibits a suppressed reproductive capacity.

37. (canceled)

38. The use of claim 35, wherein the intron-exon boundary targeted by the genetic construct is the boundary between intron 4 and exon 5 of the doublesex gene, optionally wherein the genetic construct targets a nucleic acid sequence comprising or consisting of the nucleotide sequence substantially as set out in SEQ ID NO: 2, 3 or 4, or a fragment or variant thereof, or wherein the target sequence includes up to 1, 2, 3, 4, 5, 10 or 15 nucleotides 5′ and/or 2′ of SEQ ID No:2, 2 or 4.

39-47. (canceled)

48. The method according to claim 36, wherein the intron-exon boundary targeted by the genetic construct is the boundary between intron 4 and exon 5 of the doublesex gene, optionally wherein the genetic construct targets a nucleic acid sequence comprising or consisting of the nucleotide sequence substantially as set out in SEQ ID NO: 2, 3 or 4, or a fragment or variant thereof, or wherein the target sequence includes up to 1, 2, 3, 4, 5, 10 or 15 nucleotides 5′ and/or 3′ of SEQ ID No:2, 3 or 4.


SEQUENCE LISTING (94 sequences)

SEQ ID NO: 1 - DNA, 94797 bp, Anopheles gambiae
gctaatttccaagtcccaaatgttctggtggtatattcatttcttataacaagaacccgttgtttatgaataattttgttaaattactataattttatccgatgcaaatagtaagaacagatttttggtttgcagtgcttacagcacttctcaaaatattctcgcgggccgcattcattatccacgtgggccgtatgcggcccgcgggccgccagtttgacatacctgcattaaaagaaccgtagcgttcttctcttgtaaaccggttcattcatttttttcacgtgaaccaaatgaacggttctgattcatttggcacacttctagtacagacaaactttaatcgacaacagttgttgtgccaatgaagaaaaataataataattataatattaataacaataataaaaagtaagtagggattgtctgtaagagtattttttctgtttatttattcgtattgaaataatctaaaaactattttcaacttctttatggtttaaattcttacctcttccttttcaataaacaaagaaaaaacagttcaaaataatattttatttacaaataataaccaaccattataacgaaagcgtacagatctcttcctaatgccatcggtttgacgcgcatattgttacttgggacccttgcctcacgcatacataacaagcgagcgcgtaaggctgtgctctagcatatggaaccgtgcgtcgaacactctatcgcccatattgtgctgcgttgggaaacaacctatcttggcctttggaaaaccgctttctggctgctcccggaagaacaccactcaaacatgcatcgcgagcaaataaacacccaatcgcacactctacaacatgcacgtgtttgaaaaagaaactcgagccgtacgacagtctctagttacagcacagcctcagtaacaatgttgtgaatgtattgcagggacgttgtgttgtggcgcagtcttttttttaaacaaaaccgaacccttagtgtaaaccgaacgtggttgtggggatagagcgttagaggggtgggcagggaagggtggaaaaatcaaaaacttgttgcacactccgccggaccagaccgttgcgatgtgtgtgctgacctacaacaactttcctttcccagccctactgccccatcctaccgaaccgtccgctccggtgaggcagcgtgctcatcgatgtgtgcgagctgaaaagggccgtgcgcgtgtgtttgtgcgaaacgtatgtgtgtgtgtgtgagtgtgtttgcgtaaatgcacatttatcagtgcagttccgcgtactcgccgcttcgcaatcgcaatctggtctttaatcgaggaggcaacatttgaccatcgctcgttggcagttgccgtttactactggggcgggtgtaacgaggcccacaacagcagcacggatcttgtgctttaacggtgagacgacggtaaaggtagcgcaaaaaataatacacaatgtgtgcaaagtgcagtgaaaacaaaagcgttatgtaggtgttttaagcaaaggttctacaagtgcgtataccaaagttgacaaagtgcgcgaaatcggactctgccaagaagtgccgggaacaaaacaaaacagctacaacaacacaagcaatcgacacacacacacagagatgtgtcgtcgtgagtggtaaagggcagtgaaagaatacgaacgtaaagtgcgcaaaaaaaacattcaattttcagtgcgaatttgattattcaacgatgcaattgtatttgaatgtactgccggttttgcacttcccaatacacacaaacacacacacacacacacacacacacacacacacacacacacacacacacacacacacacacacacacacacacacacacacacacacacacacacacaccccacactgtcgttcgttctgttcccttttttgtgaagtcgagacgagccactcgagccgtcaaatggcgaggacacgcacgtgtgaaggggaagagcggtgtaatggtaatgagactgttgtagcgaggggcgggaggggagggtagatgagagtagaaagggggaggaagggcgagtgctccattggcgtcgctgcatccgctgcagcgcgcggtgtgtgcatccaagacgttttcgcttcggtcgttcaataataaaaagtgtgcatcgaaaccgcacacacctttcctctcctctcctacgatcaacttctctcacacactccctctctctctcttacacacacacatccactcgggcgaatcagctccatggggcgcagacggctcttcgatggtgtgtatgcgttgcgcgccaccttcacgcacacaacgaacccgctccttataattaatgcaacaatgttgctccgttttcattacctgttttgcttcccaccgacagcaccgcgctgtgcctctcccttcgcacgccctctcccccccccccccttttttgcatcgttacccctttttgcgtcgatgcacttccatcctctctctctcacacacgcactggtatttctttctcccctcccgttgctgcaacccacctcaatcacccccccccacaccctttcgcacacttcgcctacagcccatccaactgctctaatgctaccatttccccgtttttcgcgtactgctgctgcttcggttggagagccgcgtgttgtcatggtagcgtttgcgtttggccgtcttttttgccttcatcttttgcgcccgcgtgtttgtatgcgtgtttgtcacgcatgtggtgtgtgtgtgcgtctatgtgtgaccataaaaaagcataacgcgacgaagtgtttgctagcaggcggcggcggcggctcgctgggcagtgtcggttcgttttcgcgttttcgttttgacggcttgttagggcgctgttcggtgttgttgtggtggcgccgtcggtgtacgaaaatcaaaacaacaaaacatatgtttttcggaaagttccaccccaaagggttgtgcgcgcacggagcgccgctcggtggagcgcattgtgtatctgtgtgtgagagaaacagagagagagagagagtggaagagagggggatagagtgtgtgtgtgtgtgggaggcagaggcttgccgccaaatattgttgcattctgcgtggcattgcgtggggttttgcggactggtgaatatcggtgtgagcgagcgatcgtgtgtgggagggggttgccggacggccggtacatttatcaaacgtgagacacgtgcgtttttttgttgtcgttgttgcgcttcatgttatctgtgtgtcgcagtgataaggttcgagcagctcagcaccaattgcactgcagagtggtgtgcaaaaatcatgttcgttatacctacgatgaagttatcagtctggagagaaagatgcaattatgttggataatgttgattatttatctaacgagtcgtgtgacgatcagagctgataaaaaacactagcagactatcatttcaatcagcttaatttatttcatttctcactgttgctagggctgtttagtatctcttctatttgtacatttgtcagtgtagtgattgtaacgaatgatttaatcaatgataaatgattgaaggaaagaatcgaaaatgaaattattttttcttacaagtatgttaccctttttcatcgtcatttcgctcgcttggattacagtcttactctttggtatagttatacaaactattataactattgattataaattgaaattagcataatagtattatttatcatttttctgcaaatattctttggatagattttttttatcttactttgatgaattatgttttgctcattcattatttgaaaatgtggcaacagcttgtaacagccgttaacttgttgcatagcaattcaattctatactttacaaaagggtaagattgtggcattaaaatctatgtacggtactcgcaaaccgaaaaatttaaaatcatttcgattgtacaaagtacgcaattacactcttttttattcctttacataacttcctatcattttcgtccgtttcatttcattgcttgttaaatataggttaacacttcgctcaggatccgtttattgtattgtattctattgtactaacaccagttttaacaccatttttccattccttcctgagatccttcgaatagtgcgaaatttgatccttgagcggtccacttgtctcaccgtttatttctgctaatgttcaccgaggcacatatacacacacacacgcccccggacacacacattgatagttcaacccttgtctgaatgattgtaaacgcctcgtatcaccaccggggcgaccccatcccacattgactgccctttgcaaaaagaaaagagaaaagtactcactctatccgtgctaagtgcaacagtgtgtgtgtacaatacgtgtcctggtgtgagtgcgagtaagcgagagtgggaaagagacggcaaattgggggtgcaaaatgtgtgagtgtgtgtgtgtgtgtgcgtttgtggggagcacgatcgtacatgcatacacgtgctcggtcgtctccatcacgtacagtgcgcgcatgcttgtgtgtgtgtgtgtgtgtgtgtgtgtgtatgtgtgtgatggtgtgtgtaaaagcagccgtgaagatgcagggttcgctgccgatgcaatgaggggggcacattgagtttgtgcgaaaatgtttgccaaagctcgatcaaaagggcagcagttcgttcacacataccatcgcagcgttagcaaacagccgccactgctcaccctgcccgccctacgacggagacgagcggcagccgacacgcggacagcgttccccgtgcgggtatggggccgacgcgacgcgctgcgagtgtatgtgtgtacgggcgcgcgagcgagacggacggcgaacggtggcgcgcgagcgagacggacgattgacttcgcctcaactctgttgcattgcgtgtcggcgatgcacttggcgaactgcagtttgttccgcagcatcgttcccatcgcatcgcatcgcgcgctacaaccgagacgaccgtagctggccacggacgagcgtcgggaacacatacaacactcctgtgctgtccgccgtcgacttcgaaaggcacccaaatcgcgctcgctctctctgtgtgtgaagcactgcagaagcgtgcagtcgacattcgagcatccgttcgggcagtgcgtgtggtacgtgcggcagtgcagtgggccgccggtaaaagtgtatatcgttgctatgtcgacgatcgcctactaaggaaattgcgtccaatgtaccagtgtcagtaacgcgcgtgtcggagaagcaaacagccacggcgaacgcaacggaaaaaaaacgtttgtaaccgcgttagttgaagcgaacgagaactttagtgtgttgggcaggatttctctgctaaaacccggaaactttacgttcggatcggtgagctgtgccgtgtgtgagaagagagccttggcggtgacggcttggctgagaaaggggccgcccaataatcctgaacggccgtgcgtaaatagagatagccgtgcgcgtgccggtgcggtggaatttcgtgtggttaaatctgcttccaataaaactcgttgacggcgcttgacaaaatacagccgcccaatcggtagcagcggcccagtcagtatcggactgcaaaaaaaaaactgccagttttgatagtgtgaggaagagtgcggcctacgcgcacacgtgtagtttacgccagctgataacggtttcggcggcaggccccaaacgcacaactcgcaggcggtacgcaacacagttccaagtcaaaaagcgtgaaaaaacgcctgcatccccaacaaacacatacacgcatgcggccgatagaaaagtaaatattcaccaccgcctggggaaattgcgataagtgaagggcggtgaagacacggcacagatattcgattgaccgcatatagaggcgcgaaaagtgtagaattaaatgggtagaaaataaacactccgcgttgcgttgtgatgtgtgatgtgcggattggagcgagtcacaatcctctggccctgcgcccgttgcagtgaaacccgcgtggacggaatgcaatttttatctatctcgtgtgtgtgtgttgaaggggtttgttgaaactggaaaatcaattgtgaaacaaaaaattatcagtgattgtgatggtgtgtttttgttgtcgttaacagtgtgctgggaatgagattaagatttacgtgtgcgtgtagtacttgcctggcgagcaagaagatatgagatacccgctcattcagtaacaaaattagtgtgatcgtgtgtgttttatgtgattgtgcagtgatgattgtccaattaacgtaaagatagcagatttaagaattttatcaaaaggagtgcttcaaaaatatatatttggtaagtaaatatgcaaacttttgtgaaatcctcctaaggacagtcaggccgtgtcgcttgaaaaaagtgtatattttccagggaaatcattagtcatttaatgattgctagttttttttttaatgtaaaattaaataaattctattaataaataaattaaatgtgcagcatataaatgagataacgaaattatttattttctcctgacatgaaattttgtaatttttttttgcttttcgtaaccttaactatcgagaatttttttttacaagacgttgactaactctaacgtttgtctaagatcgtaatacacatcgcaatagaatttggtcaaaatattccacagtgatttaaatttatgaatgcgttttgctgatacaattctttaattgttgttaattctataagtattccaagtcgtactaacgttttattatccataataattccgttaatttggtttcaatgcttttggaatttcaaataagctatatccagcattaatgaactgaaaaattcaataacacaattttcattattttcaatggtgttatgctttggtcatcctagcagaagtgaaaaaatgctaattttaaatgttccaatgttttgaaatattacaggaaatcaaattaatgtatattatgtcttaaataagatgttaaatggacaagataataattagccaaaatattgcattacttcaaataaaatatgagatctttgaaaatacccccgtgcaggcaattggctacagcaagaagcaattgcggttctttgtcattgaagttatatatatttaaaagatatatcaacaaaaatatgctttttaacatttgttagatacatataaacattcgagaacaatacaaaattatgtaattttgaattttaacaccataacaaatgcaacaaacatagcctgtgtgttttgttttcttaacatttttttgtcatagtattaaattatttgaaatgatgtatatgatcccttcgatcgaattctaatgacacttgatcgaaacaaataaaatataaaatatatatagctaggcttgtttaaaatgttttatggtgagcgaagatctagtgtgaccttaaattataaaacagctatttccatatcaaatttcattgtttttttttttaatttcaaagatcggccatattgctattcaaattttcttttattctgaagaaatgccagactgtaatgttcttacttacattaattatcatgttcattatcttactgtcatctgttacctgtattaggtccggttatttaggtatattgaaatgttaaatgtaattttacgttggaacgcctatatcatcttaatgaattaagtttaatatgacaaaaattaagaccataaaatttctaaatggttctttcggtacgtttgattgcagatctcccaaaccctagcaccatcgcttcctcgaccaaccaataccgacagcccgagaacgatcgtacccgagtggaaaacacattgtattttcgcagcaaaaacaacacagaaatctttaaatattttaagataaactccatgtcccgacaaatctgcttttttgcgattacatagtaaagaaacacagtagtgaggagcttacttttgctcgtgctcgtaccaccttttaaaaaaacccggagggacaatgccgtcacgcaccacggccaacgatttgcgcgagctcgatgtagcgccggcaagtgtaacgttagatcaagcttccagatgttgagagtcggagtcacaatacgtccacaactgtcggttcgtccaatctgtacattgtgtggtcggtgtttggtgggaatgacaacggtgtgtcctcttcgaaggtgctaaaaggaagctcgctgacgaggcggtagggtgtgagagtttggccagtttgttgttgcgcttgtgtggggtgcagcagggaaagcattagccgagaggtagagacacacaagctatttgggaccgtgaaatacgccgcgcgcaacagtaataacataacgtaccgtaagccgaagcgatcgaatcgtgtaatcgaagcggtctcgtgtttttttcctcctatatcgagaggccaaccgatacatccaggtgcattcggcggcatagataacgcagcattaagagtcggaattggctctcgaacgcaacagtttgattgatatataggcaaggcgtagtcagagaggtgctgtaaacgagaagaaagtaaggctagcaggagaagcgcaagttgaggaggggtgtcgcagggttgacgtagacgtagagcttgtttggaagacatacgcggaaccacacgggcgtgtggtgcatcttgaatggtgtcacaggaccgctggacggaagcaatgtccgactccgggtacgattcgcgcacggacggcaacggtgcggccagctcgtgcaacaactcgctgaacccgcggacgccgccgaactgcgcccgttgccgcaaccacgggctgaagatcgggctgaagggccacaagcggtactgcaagtatcgcgcctgccagtgcgagaagtgctgcctgacggccgagcggcagcgcgtgatggccctgcagacggcgctgcggcgcgcccagacccaggacgagcagcgggcactgaacgagggcgaggtacctcccgagccggtagctaacattcacataccaaagctatcagagctgaaagacctgaagcataatatgattcataattctcagccgagatcgttcgattgcgactcctccaccggatcgatggcgtccgcaccggggacctccagcgtgccactgacgatacaccgacggtcgccgggcgtaccgcaccacgttcccgagccgcagcatatgggaggtaagtacgatcatgcgtcttcatttcttcgtttttttacaactgcttcagtctgttgaggatttaacacactttttcatacatatttaccattgggatacaaactgaggctctcatagagcttcttcgaatggttcgaatcatgcaccgaaaacacttgcaagactatgatttgctccaacatcacgcaaagtggatcatctccaaagtgagcgcatctttaatgcttagattgcgcaccagagatcctccagttcccacggattgggcctgtgctacattttattggttcgcttaggcactgcctcaaattggagcatctcagcacggtacgcacgaggaacggctgcactcagacaacggtcggaaatccgtgcaatcccgggaggggaccggttttaatgctgtttggtctacgttgcctcgctaaacctaccttccgggatctctgcaacatttttcgctcacctgccacttcgttagattgtagttcccgtcgcgaggacagtgccgggagttcggtggagcaatgcgctaggctccagagaggaggctacgaatgccttggaatggacgctacacactctttttgtgcgtacttccaccacacgttacctcgacgattaccctggtggcctggtgtgcctggtgtttggcgtttacgtctcacttcgtatgtgtttcacccatcacccttcgtttcgttgttgggggctctgctttttttctgcttctttcgtactccctctcacaccactgctgcttgctccagcacgtccgattcttttttcgcatcgtattaccataattatattatttaattatctacttcttttcgaacggtggcgttggagcccgtccctctctctctttttccctcttttccctctctttgtctggcactgtgttcgtttgttttacttgtttgcacgcttggacaatgcttgtttcttatgcatcatcccccattggtacattctttagcaagacgcgtatcctttcgcctgcatgcagaaccgtttaagtgcgcccaggtccggagtgagacgaaattgatcagaattcagacacacctcgttatggggccgatgatgtaccgccatgctgtcggacgcattggtttggcgacgaaggtgtttcggtgccctggtactacaaataatggcaaacggtgcactggcgtatgcgtatgcttcttcgccccggttcgttttaaacggatcggtaatagtaaaacaacacgtaaaagcgatattttgtagtggactttggtaaacaataaggttccggctgcagttggatcttgtttttctagctacggaatgtccggtgtgcaaggcagacgttcttcagcaggtcctgtgcgtgataaaacacaaagggacaaacttttcatttgctcctatttgtacaactgcgtggaacacacctcatatacacgcacacagggtacccggggaaaaatgtcgtgtcgcttccttggacgattggtatgtattcggaaaaagaaaatacttttcgagctcgtgtgccgggtggcggtggctgccgttgttggaacggttatcgccaaattgctcttaactttgccacttgtgcaattattacttgttatatcttttcctgccggctggcttctctctatttcccccaacctactctccctttcccttcctttcctctatcgccgccatcatgccaaaggaagctgcagtcagcactccctactatcggttgaatgtgtgtagtcaaagattaagcgttgcccgtatatgctaaataaaagtttgcacgcaattccacgcttttcctcgccgcctgcgaacggtggggttttggtggcggggcaatgttttcttcctgcacgagaggacgattagttgaccttactgagcgcacggagggaacgcaggagtgtgggtagggtaggttactgaatgaccacgtaagagacgtttttgctttgttattgattatttttcagaggaaacagaacaaaatgagcaagttgaacatttgatttacattcttgggctgtgagattgcattagatttgtgttgagctgttttttgaaatgtaaaattattagcaattactgaaggtttgctgaaaggagagctgaagaagtattctattgggaaatatatgtctataaatgtgcaaaatactttcccagaagattcaaaaggctcggagaaagatcttacattttgtgttgtaaatgtgatcattgaaaacctcacaacactaaatatacctagtaaatttaaatttttaacgatattgcctacataaaacatctagagtcttaacatcgcttagaaatgccgtttggtcccagctaccaacatgccaacacgggtccggtcagcaccaaacccgcctatggaagctcatctttggcttgtttttattgttttcatcccctctaaaacacattcccggtgcggcatgttaaaactgtcattagaagctttggcgcgaatcgcgcgcgcccgctcaggggtcttgcaaacccgttcgcttcagcttctggctgtgtgtgtgtggctgggcgtaggtacgaatttgcggaatgttgcagaatgtgtcgccagcaggacagtgcggtgcggtgtgcatttgctagaacaggtttcgcgaaggaagaacgtttgctagctggctgtgtaaggcttttgaaggtatttgattgattacgaccgccaacgttcatcgttaatcatgcgcccgctcagaatagcctaccagtcatgggtggaggagttcgcggtggagttctttccaggcaaagcagggagctgcgtgtgacccggacccgcttgcacattgttcgacagccgcagtcgctccatcgaatgtccctggctttgctggccggctttgcgcaccggctcgctctggcgcaatgagttcaattttcgttgcgatcgtgaaaagatcgcccgaatcatccggtagtctgctccggtgctgcaactacttattaagcagcattatgtatcttacagctcattaggcggcgtcgaaggagcacatcagcaaacaaccgtaccgtaatgtcttaaatgcgcgtttatgatggggtgacggacctgacggcatggcggccgttgcttttgttttgattttgtttttggcacttataaggtgtggtggggttgggcggatggggtcccccaaacaggtaacgactttgaccgtcgccgtaactggtcgctggtcacatgtcgaaaggtggagggctgcactatcaaatgtcactgcatcgaaacgacgggaggtgttgtatgtgtaccatgttactgtttgtgtgtgtgtgtgtgtgagtgtatgctggccaatgttgcagaggtttttgcgcgcgtacgatcgccctgtaaccggtttgaatttttgcacacatttttttgtgtatttccagcatcaggtcgcgctggaaaaggtgattcgatcccatttctcttcgctccaaaatcgagcgcatgcacctcggtacgcggtatgtgtgtgtgtgtgtgtgcttacgtgtttgatgggtccggttactgcgcacataaatcctcgacacagtcggacaagggctctcgtgtctctagtttttggcgatggcttttcggccgctcgcgcgcagctcctgacggctccgagcggcgatggtgttgattgagtcatttactaccgaagcaccgatagagatctcgttggtggtggtgtgcgccacagatcttgacgacagattttttggcgtccgtagaagctcatttcacggtgcgatgaagacgaatggccggctagagagcgccgagtcgctccgagcggtattgtggtcagagtgagtagctttgtcaaggcgtcgttaccctttatttctctcgcgatcttcgttttttttggttaatcaagaaggggaaaagaatgacagcaaactagctgtttgagaaaagcggagggttggcttagcgacaagggtgctacataaaaaaagaaacagacaaagagcgtgtttaatccgattgttgtgttgtttccggttgagggaaccgccatgctctgccttccaaacttccgcactaaacaacaacttcctgcgcatgaggactatcactgccgcaaggcgcacatctgaagaagcccaaaactcgtcgtcgaaacaccccaaatcaaaggtcaaacatggcggttactgcttcttcttgtaaggccgccgtcgtcatgcttttgtgccgtacattgacacctcaagtaaaacagagcagcggctagcagggacttttgatgaacactttcgtcctcgcctgatgagtggtagaggcacgcaagcatttcagtttttcccctcctgtcgaatggtttttcgccccatgcgaaaaatggttacagtgttcgaccgtgagtgagtgatattttaaaagatatttcacatttactgctgctccctttcctgcgctgcgacgagcgcactcgctcgtacatcccattagcgagcacgcggccctaccaatagattgcaaatgcgcctttctgcgggcgagtcatgagtgagacatctatgacggataccatgtggacaaagcgtaaaaaatgcacacaaacacacacacacacacacacacacacacttgcactacggcaaagatcatcttttacgcgcaccgcacaccgatcgcggcagcgcccaaagtgcatagcgatggtggaggcttgcgttttggaacagaccgcgcacacgggccgccggtgtgacgtgtggaatttcagctaattagaaaattattaatagttccttgcgcacatgatcggtgcgccattcttcttcctggccaaagtcacccgggttctgcatttccggagcagagtcctcgacaggttttcactttccctgtcacacgtttgagtgtgcctatgtgtgtgtgtgtgaccccttctcgtcttgtgccttggggtcggctagcaatttctaaaacttgctcaatggcgcatccttttcctctctgtgcggagaacgtttttccgcgaatccatcccctcgccccaggtgcttatgcaatcagcgctgctttacaaattaaaacgtaatttagatcctgttcattaaggcgcgcgcccgatgcgatcctttccccgcgccacgcggtgcaattaaaagcgtatttgaataatttgattattgtatgaaaatcaaagaaatttgtctttaccggcaacaaaggcttggcatgtggaaaaccagcacaccgacagaacaggcctgtgggaaaacggagaacacacaccggcacaccaaactggttctttccgggtgcgcgcgcgacagcagattacatctggtgacacgagataatttccattccgcgatgcgttttgcgctgtttggttgttgtgcgtgtgttcggccgaagaggaggggggggggctttggacagcaaatggcttgttaatgggcttttacctttgagaactgaaccgcaaaaccctgccgaacaggggtgagtcttgagacagtctatcgtcgaagctgctgcgcgttcacttcctcatcacgcaagctggcgcgcgcacacggcctttattttggcagcttcaatcggaaagccagcacacacacacacacgttcgacagctaacgagaagcagggttgggaccaccgattagagatgtgcaatccgcgctgtgcacttttgcatcgtccacacaccccgcggacactttgctcgcttttcgccccgttgttctcggttgatttcgccgttcggccgccgacttcgattccctcatacgggtggaaaccgaaaataatgcgcgagttgcgccgccacccgcctaaatttagcaccacgagccggccgcgagagcggcaacactgttgcgcggccaaatgtctattttcgtctaattccgcacagcccgtcggtacgctaagccgtattgcggccccgcccccgctgtacccgccgatgccgatcgcggagcaatgtgcgcacttcttgagcaactagggtgcacttgcacccctgtcgtactaaccttttccgtgcgccgtgcgctctcgtgcgcactgttcttcctctctctctcacacaagcgcataaaatgtgcagtttgcgggacagatgtgtgtgtgtgtgtgtgtgtgtgtgtgttgcgctttccggttcgttacgtgtgacgtgtgtgcgcgcgcgccattgctaaagcgatcgattatcctccgggagcgctgttctgttcgctcttgttctttcaattttaaccaaccaagcaacccacccacccacccaccatgcaccccgctgcctgttccacatgtgcatcagtggtcagcttgcatgctcgaatgcagcaaaaaagtgcaatgcagagagtgcagcaaaaacaaagcacaccatgcgacaatgcaaagatgtaaaagtcacacacctccaacgaaccgcaatagatgggatggcccctgctgggacgggcaacgggagaataggggcagcgatgatgattgatacattcatattcgtcgccggagaccacccgggccaccgtggcagcccttgggggggaatatgagcatcgcgtcacgtcgtacttaatcaacgcgtgtgcgttatttgtctgcggcacttccgcgtgcgtatctgtcgtgtccgttcggttcggtcggttctcggttggccgtcccggtgctggacacacgctttgcgcgattgcggacagtctgcaaacggcaacggtatggtgtgaagaagtggttcttttttgtgtgcttcttttctttcggaaatatgaaatttcttccgctgcctgcctggacgccgggaactggacgaacacaggcgcggtccgccgtattttgccattttcgctcggatgtggtcggatgtggggccaattgcacacacaaaccgcgcgaggtggaatgtatttatttacgttttaacggtgcagctgtctcctgccggtgcatttcgtgaggttccttttgcccatcgggagtgttgtgagaggagtggccgaaacaaaacggaccgaaaaaaactgccacagcaacagttcgaaaagcacggacgcacaaaaacgagatcgctcggaaaagtgcaactggtggcgatggtgcattatttcacattcttttggccgtacgaataaaaacatgaagcaagtaccatgcgaaaattgaacttaaaagatccacccgtaacggttgcacggcagagcgtgcccgagtgggacgtgcgttaaggtgaaataaaataaattaactacaaatttacaattaaattgattccatccattgcacagtcgaggtctctgagcaggagtactaatattctaccggcaggtccgtttgcaggctgcaacaccgtcgtgcagctttcccctcgagcaggcagttagtaggcaaagtttatgtgctagatagcggtggttttgcggggagaatcaagtctagcacacacaaacaaacacgggtatgtaaaggttgaaaggctgtctcaggggaccgagttgccgattgggcgctggttcgtccaccgtccatcgcgcgtcctgaacggaaacaataacactcataataatgtttcaattaaacacaggcgggacgacgacaggaaccggttatgatgggacaatttcacaattgcacttgacattgggcgcagaattggtttgcaccagccatccagggacagttgagcattgcccagtttgagcctttggtctggagcttttacatgctaattagatttcagttagacaactctgcgcaacatacgaatgctttcaatatgttgcacaagggcacaatgccgcaacaaggtaaatgtttcctgtttctataaaacagactagacgtactttaaccaagctatggacagagtctattttcggatgtcataatttacgtttgaatgatcaatcacatttagtgactgctaaacctgcttgttatgcttatcctgtgtatcctaacgcttaattgttccgttgtgtcgttaaactagcttaaagcttcttgaaccattgaagctaccattatgaatgcagtataagcatgcaagatttatttcttttcttcgtttcgattattctttcgtaaaaggcatcttgatttaatgaatcttttgcgataatcggctacacagcatggcatctgcggggcagaacggtactcgatcgagcagtcgccattatctaggagtgcgtaatcaagtttaggttgccacgtgattcgattcatttcacaccgacatgacagcagaatagaatacgggtgcgccttgccgcactaccgttgaccgtcgcgcgagaccttctcaatggctgcattcatctcgctgctcgcaagtgcgccgtgagtggagcataaatctcgacaaacgttattgcatttcatcgactgtcttcgatcgggtttggggggggctgggtagacatttaggaagcaataacaactgtcttatcgtgcaaggaaacacaccggcacgcggctaagcctgtggtgcagtggtttagattcctttttacttttacttaccaccgcacatgctttatgttggatgttcaacaggcagcgcagacaggctgagagcggtacagcatacacacgccgtcttgcttgatagacaaggcttcgcggcctggcattgccgtggagtgacgtgtaagtagtgccccaaaggcaccactcttcacgggatagaattgagtgcgttgatgtgaacggggggcgaggaagcgtagtgccggttgtcgtcgtagttgcagcttctgcccgagcagcactgtcaaaatgggttttgcgctaggttgagaatcggaggagggccttcgccgtagaagccgtagcgatcgtcctccgcgagcacgggacgcaatgttgccacacattttgccgcgctttttttttgcactcggcagagttacgacggctctccggtatggaagcgagcagcacatctcacgggctgcgtcgaaaatcgagcataattgtatgctgtctgatctatttcatttcgcgttttatgttttattcgacttgctgttttccgccgcccggctcagcttccaggcagggcgggaggctcattgtaggttagggccccgtttgacgtgggccagacagtcggcgatggggcgaatatggggagaggttggtgaccgatccctactccatcgtgtcctccttgaggactagtttcgctctccgacactcttgacacttctcttccttcgtctgatcctctccagggaaaggctgctgggcgagaaaaccttgagacgcgggagcagccagaaaccggctcctcctgtgcagcgtgcaacaaacaaaacagcaaaagattctaggctccacactgtgcactactacgagagagaaagagtgtgtgtgcgtcctggggtagttctgtcaatgttgaaaaaggtggcaatggaagaagagctagaaaaacagaggcattatggggtgtttcaggcaggaggattggtgggtgttaggccgggcaggaaaccggatgggaagtcgaacgggatacggatgctgctgttacgccactgaagcggaatcgtttgcggaatcggtcaacattgttgagatggccgtgttcagcctgcggttgatttagttactttttgattcttttttgattcatttcgtttgtgtgtccaaatgaagtgtgctgttgggccggcagatagggctttcggcgggtacgcactcgagagttcgtgcgcgtatttctcgaacgtcacggcataccctcatcaagtgaggctgtcccgcgataggtcttgtgtatgtgtgtgtatgtgtatatatttttaaattctggtttggggcatcaggaccctgaaaatgtaccaccgaaacccaacggagagacgagcttgtctgagaatggttgggagcgcaagcagtggtgcttacgatttataaaataaacaacgacgtacggataccgtgcgacgggattaaggtcacgttcaatgttacgattgtcgatcgagacaggcatcttaagcgggctgaacggcttggtcacactggaagggattatttaccgatataagcgatttcaccattggcgttgtccgtaatgcgagggcgccgataagctgaccgaagcaggcgcgaagagtatttttgtaacttggttgaagaaacaatcacaagcatcttgatgataagggataatgaattaaacataattgcatcacctgtgatgagacagttgataaatgggacgtctcgcgaaattctggaaagcgagcaatatcttcgtacagctgcatctgacattgacgtggctgccggttgcattgcgaaacgtcaaaggtggcgctaaaagtacatgtttaaaattagtttccattttgtttgtttgtaatgcgctccggtttgtgtgcatgtgttcgggtttttagctattaactgcaatttctgcactgcaaaatgtagccgttccggtatgatcagctgcagacacgtggtggacggatcttctgcttcgcgcaaagtgcacttaaatggtcgtcgaaggagtggacagcgcccgcgtctgagctcataatcggcaggccaattatgtcgacgggaatgtggaaggatgcttgctgcagcgaacaagatgcattaagcatgggcaatcaatcatcccgtggctctgcaatcgaggtttccgtgacacacacgcgcgtccccgggtgtcgtcgctgacgatcgcgtgttttacaagtgcgtccgtgcgttccgtacgtccgctgcgtcgccgtcgtccgagccacaacatgcccacggccaataatcagtataattcggtttaacgtttggttagattatcgggaaagaaaataagccgaggtaaaaacggatcacttttcaaaccgaaccgagcgcaggactgcaaagatgggaaatgtgtgttcacgtgttgcgtgcgtgatccagggtgtatgttgcgagaaattattggaatcattccaaagttatgtcggtaacctcagcgtttttcgtgcggtgtgtcggttttatgcagaaagcagagatcttaaagcgagctggcattttgatatagcacatatattcgatggatgtagcattgaggtatcctcaatgaccattctaaattatcttatccttaaggctgtttttgggccgagtcctgcaagactagaaaaagtccgatacctattctaactgtcctcccatgtacacgtttctgcatcgttcctggaagtcatggaagtcatagagagtcattcagtttcatcacagaaacgaacagaacattgccatcaaattggacagtttcaaaacttcattcaagcaaagattaaattctagcgttagctccataagatattcgacctccaggttaagttatattggtctctagctaaggttgatgtattgatatggtcttcaaacctctactacaccctaaatatctttgtcaaagtcgttaactctcacctggcatgtagaggaacaggcaacagaccaatgattgaaaagccacgctcatgtcttcagaccataacctcggccaaatttaccttccaatccatcgataaaacctcatcgttaatgtcattaaccttttgcaaagcttttactccagtgccaccaacaaacattgcgtcaaaaaacgaccagtgtcacgttctcctccctgtgtatcggagcatctacgaaaaaaataccaaaagcctcccttaaactgggaggcccataattccagctgaacgcttagattggaacggaactggcggtgtctttcgtagggctcggaacgttttcctaccagcttctgtttgctcgaacccgaagcagagcacaaaccgtctaggttagctgacagaagaaattgcaagatgcacaaaaaatcgcacacacatacacacagacgttaacagtgtattgcgaccgaacgggcagcaaaacgctgtggctattgtgccagaccagaagggaggagaactcaaaaacggtaaagctaataaacctgtttctttccattttttgcgcattgattcatttcttgcgccggcgagagctgcccggcagttcctgttgcatacatgcagggagcgcgggtttctcgatgtgcgccacctctgccgccggcatcgccaccaccgtcaccacagaccggctcgaaggctgcgggatgcaagcgcggcaaccactggaaggtaacctctcggggcgattgttgtatttaccaatcgtgatgcatgatcaatgttgtgcggagtattttatttcttgtaagcagcagtttgaggatcggccagaggtttgggtaaacatttcagtcgctcagtcgctcgcgaaacagaataaaaaaaacgcacacagcgttcaagagaaaggcgcgcatggcggtggatgtaaaatgcctcatttgtggcgtcttttcccctgcgcgcagcagaacgtgaatgtgtgcagagcatggtgtagcgtcggacgaggagcatgaattttgagcaagcggagatggttttgagtaaatcggtttctatgcagccaaggcaacggcagccgcatagaactagagcactgtgggccaagtcgcagtcgaggcacggaagcagggcagaatcgcgactctctatcgcccttgttggacgacggataggaccgatgccggtgcgggtcaagttcagttggcttaccgatgcatcatcggaagccatcttaagtaaatggagagctggttggcgatggagcatggggctcgctttactcttttgagtgggcacaggagtgttgtgctagaaatagattcggctcaaattacggctcgggcttgcctagagaaagggcaatgaaggattgaacacatcaaagttaagtattttttgtatttgtggttgctgtcgttaaatggtttattgaagcgtttccattataaaagttgtgaaacagttggaggatgaacagaaaagcgtggatgtggaattatatttcaatacaaacacattgcacatgatcacatggatcaacggtatataatttagttggatataaaaatgcacatccagcattgaggatggtattttgccatcctccacagctcattatgttcacaaggtgatggtggcgatggtttcacagtaaaagtttctcaggcaaaacggctgcgaggcattgtgcgaaagtttgcagtaccgtgttctatgttcacaattgggttttaaatgccccaaactgttcgaacccttctcacatggagtgtgtgtgtgtagctgtgtgtgtcaaggaccgcaaacaggaagggtcaagggacaagggagggcttgtgatcggaagcgcaacagaatcatgatgagcgcagactggcaccgggcataatttgcccgtttttttatcgtgtgttgcgcattacggccctatgttgaaggagatcgttttcctccccacatacatacacacacacacatcgatcgtaaggtatgcaagaggaatgttgccttaacactgcgcgagttcggttgcagtcgatagaattcggtggtttcgagtgcgtgcagcgcatattaacgccaaggttggtcaagtcgtttttcaacgccccttgaactttggtgatgcgagtcaaggaataagagcaagaaaacaaacactccacagaactttaggatgcatggacgctgctgcagtggcggtgatggtgctgttgtttcgtgtgtcactgtaacacggctcattaacggctgcagacacagcgattgtgtcgtctgacgagtttactttaaattagcgatggcaaaatcaatagaaactttcgtcgccgccgccgccgccgtcttttgtattgatctcactgtccagcgaaacaaggtattagcacgtcacgatcttatcccgattcctgatcgtgtaaggtttacttacttttaatgagcctaaaacaaataggaacaatgctcgtcggaatgctctgcagcagctgcgtactgtttactgttagtgttcgcttgtcttgcgatgttttgcttgatcttaattattaataagggcgcggtactatttgtttgcaaaaagtcttctataatgatcgattgtattttttaaatgagatgtaaagttaaaatatttgcacaatataaacatcaaatgcaaaacatgctaaggaagaacgtaaatatttcgtgtggaatagttcctttttatttgaagttttcaatatgagtaatttttaaaaggcactttgacatatttgttttcaccaatgttacagacaatctatcaaatatgcctataattttatcagataacctgaaatcttttgcaagatgctgttcagacaatcacttcaaagtttctagtgatatttgagatttagatttgcatttaaaatcgtgcacagcatagccttttatgcattttatgtaaatcgcaatcaccacaccaaacagaggcgaaacagattgtaatattttcatttaaataacatcccccgaccacccatatgtgtgtgtaatcgagtgaccttgatgcattcagcgatgcatggcttggcatagaggggaccacaaaatcgggacgggcggtagggcagtgctagcacaagcgcagaaaattgccttatcaaataacaaaccctttctcctcatggttgcatccgcactgccctaccgcgtcgaccgatgcatccgatcgttttcatgcctgaatcagttggaaaaacttctctctcgtcggcgtcgcgaatggaaaagcgtttcacaattgcttcctactgtgacgctcgacggcgtatgtggaaaaagggtgcggtgggaggcgggatgtggagaggcttatcgtcactcactcttgggtgtatgcgtgtgtgtgttgttcgcgggaaagcccatatcgtaatcgatatgcttgttagagatccgttttgatgcaatggaaaaactaacgctccagtctagagaccaacaaacacacacacacatcgaaagagaaagggaaatgtgtgggaggaagggagaggaggggtgagagtggaaatgcaatgtagtgtgaaagtgtggctgactggttaaatggatgggaaaacaaggaaatggatggaaaggaaggaaaaaaaaaccgtccgacggttacagaaagacgcaaaagtgctcgtacgaatcgtcgtatcgtcgttggcgaacaaacaggcgaagccagagcctgccagcaacggagttctacggagctgacgggacggccagtccgccggtgtggtggatttgtttggacagaaaaagatcggaacaggagaaaaaaacgcacgccttcataatgaaatgatagacacgtgcacgtttccagtttcaaatcaatttcacactcgaagtgagaacaaacctcggaaacagtcgcacatacacacatacacattgggatggttggctggtgggtggttttggttcactttgctctccactacatgtccaacgctgctgttgctgcgtatttcatctgcccttgtgaaacgaatcaccagaagcggtttgggtttcgggagctcatgttgtgtgcgatgcgtcgccagtaagcattctcgcggaaacgataacaaatgtgtgtgtgtgttgggtgggagtgagagagaacatgaggttgggggcgaccatgacactgacctaggacaattagaaactgattgacggaaacgatatgcatcgaaagcgagacgcaggttttcttcgttttatcagacgcaggccggccttagacacgtttactctagggagtcattttgctgaggacagtgagcacagcactatgtaggttagatggggggcgtggtgggagcttggtggtccgttggatttgaagttgccagaggacaacgatgaaagtaatggccaaggatcagtgcgaataaaactcatccttgcacttacatacacacacatacggtcctgtgttggatttcgcaggacattgcgaaatgtcttcggtggaggttttactggccacgtttgatgaccttcggcattgctgccctggctgtcggtttcggttgcccggttccacatttccggtggctggctggagataatgaacatcaatttcaagaacggcaataatcgtaaaatgcagggaaatatttcttgatgcattcccgggctggatcttgaagaacgcgccgcacattggagttgatttgagcatgggaaaactcggagcgccgcccgtgccagtacggctgtcctccgctccgcgttgttacagatcctggcagttcatacattttcatcgaaccaaccagaagcatcaagccattcagccaccaccacgtaccacgagatggatgcaaaggaaggacaaaaacaaatgtaaagtcgcccagaacaatgtgcactgctcgcgcgagtcctgcttttcgtctccggtgcgtctgctgcctgcgtcttgccgaggtcgggaggaagccagcacacacacagagtcttatgccagtgatgatgcaccacaatcaatcccttctatgcagaccgaggggatcaatctaggttggtttcattttttgtttctctctcccccttcatactcgttttatgattagagagcttttccgctgcttttcgttgtgcgccgtgctgtattttgtcatgcttttgttcgacgttcccttgtcactggaccgctttttttctttcctccttccttccgcttgtttcccgtggcaggttgtttttgttttcgaacgactcggatttgccatgtatagatgcgctcagcttttacaaaaaaagacaaataaaacacgaacatacgagctaaaaacaatgcttttgatgcacaacaatcacaactaccagcgctcacacacacacagagacactctctgacgcacatttgtcgcttacgcaaagggaaggaaagaaaatgctcgaatgctgctgcagctgctgcctgggaaaagaaattggatggtcgtaaatttcgggttcggtagaaggaaagctcttccttgtttcatttacagtgtaacagtcgcacacgttggcaccacgctgccatggtggtggcgtgtggatcgaaaattgagatgaggtttggaatttttcgctacataaactttatcctgtgctggtgtggactgtttgtttctgttgcccagttttatgacgtcccggaaacgcggacaagcgaaccgtgcgaccggctaattggtctcatccgcctcgtgatttttccgaccaaccggctgcaatacaatttgtccaaccatcgtgttccgccggtggctgctgggataagcagaagaacataaatctgattgaatgccatttcaatgcaacaaattttaggaaaaatggctaaacaactccttggcaagcttctggccaagagtaaaggtaaacaacttgccagtactggtcactcttttgtccacccacctttccggttgtatgtggattgatgcattttaagcataatacattattaactccacagacaaacaaccccgaaatggcttcagctcagcttaaccaggcggcaaactgatttcgatccgcacgacatcatcttgcacgggacgagaaattgcctccgatacctccagcgcggcgtcagtcagccatctctcatatttgctctcttacaaatgatctcagcattgcctcagtcgggccctcagtcgcgcagctcgacggacagaaaagtggcgatgtgaaatattaatgttaaagaattcatttttaaatatgcaaattttaattaatattcaccctcgttcccttgtggggcaaaaacgcgggcctcgggcaacgagactctgcaggctggtagcaaggtttcggtcatctgtaaatgtgttctcgttaggcggttgcgaaaaacaggccgattttgtttcaggacagaacaggagggataaacatataaagagagagaagggttaatgtagaaacacaatatgaagttattagtgttattgctttcgaccgatggcagtagatgcccggtggatgcatcaaatcatgacttcgacaggcccaatgtccagcgacaggggtgcattaaaacaggcttgattctggatcctttaactacacatacagggtcggccagatcctgaaaggcctctacagacaagggcataaaatatgtatcacgcacgaacgatgttattgaactcatttccttttcacaaggtcaatttagtccaaagctggcatctagaaatctgatctccagccctgattgatgcaggctagcagcaaaagaaattgttttcccggaatcattcctccgattaaccatcgtgtggcatgtaaattccccactgtcaatgctgtttgaataatagccccggtgatatctcattcccgcagggcggacaggcacgatggcactatggtgaaagcctttttttcttctcacgttctcacgcgatcctgttgcataaagaagtgcactaatgagtggtggctgcgcacatgtttgcgttcgggacgccgcagtaagtcctcgttttgcagttacttccagctcgtagggccagtagcgctgcttagtccttcacggattgcgctcgatgatataatgcatcacctgccctgtcctgccatgttggttgttgttgctgcgaccgggacggatcaacgagcggtaaaattactgcacagtggcggcggtttcatgctcgcaaaggcgaatgcacaggattgtgtgcaattgtgcgacgattgcgtgcaggaagagcaggagctgaaagtgcgcagggggacaggccgcgctcgaccaaagtaatagcgggggtgtatgttttccctggtgaatgtgcggtcccacagcgttactacttcattccacttgacggaagctaatgagcagaatcaggttggctgggtgcataagagcgaaaatcacaaaagccgtacacaaaaacacacaaacagcgatgggctcggaacgggttaaaaaagaaagaaaaaagacagaacagctccaggatcctttcacgtgtacacgcaaaacaactgcagaaaagcaacaaaaaaaaatgctcctattttccggtgtgccgagttaccgcgtcggagtcatcgtgcagctcgatgtctgtgtgtgtgtgaacggtctcgcagtaacggaacaaaaaatgtcaacgagagctctccagcagaaaggaaaccggaaaattctccatcgatatagcaacagctccacttcggcgcacagtccctacctaccttcccctcactattgccccaacccattgggcggcggtggtaaatcggaacggggcatacatcagcgtcaagttcaaggacaattgtcaacgcttccgtccacaacgatccgccacccacacgtcttggggtggatggggcggtcggggaaaaaaatagaagcaaccgacgcgcaccaccccctggaagctcgcggaaaagtgtgctaggagagagagagggaggcagagaaagagagatggagagacggaagggagtctcggaaaagtgtctcggatgtgggaaatcggtttacaccgttaaccgatgccagccagatgggccatgtggggccgatgccgttcgatgtgtgcgtgcacagcgtgtttgtcatcgttgcgttgtcgacgtcgtcgtcgacgttcgtgccggctcacccatacacaggccgcaccgaagcaagcagttgggaaaacatgtggctacgacgattcgtgccgggtttttcctcgtgcactgcaacacagccctcccccttgtttccctgtcctgcgttgagtcgcatggcgcacgaagctgtttgtttgggtacgagccgttgttatgacgcggcacggcaaacgcgttttccactccgggggccggggcgctgtgtgtgtgtatgtatgtgcgcggggttaggttacgtttccgcgcgcgcgattcggcctgacgctgttcagccagtggccgcaacattgttgctaaccgggctgattttgtggccgaaagggtaggtgggatgggagggaagggtgcaatgtgcagacgggctaaaggatttggcgagacaaggaaggagtcgagagagagacgtgtccttggtgtgtggtgcaggtcgcgctgtgtaggttgagccgtctcgtgtacggttgactgtgtaagtaagtggaaagttctctctttctcactttttctctttctttctgtttctctctctctctctctctctctctctctttctatcggttgaaaattatctcgcgccacccgcatacacttgtcacgggggagtgtggggcagtgaaaatgcataccggcgaaaggaggggaaaacctcggccaagaaagggaggccagtttttctctcagctgttggttctgtcgactcggctgcacacagcgaaaggatgtgtgttgtatgccgccgcacacaaagccaagcgtaccgacacggaacacacgggcgtttgtgcatgtgggtgagcgctttggacgcatgcgatgtggaaaatcggtgaaaatgcaagattgttgctgagtgcaggcccgaaagtcagtcgtggcgcttctcgcgtacccgaaggacgcaaaaggcccgcccggtttgttgctgttcagagcaagcgggaaaggcaagatatcgtatgacacttagacgagattgagttagggcatggcgctggggtgtaacagcggcaccagacaataatgctcgtaggtatcgcattaatgctgcttgtttacttgggtttgagtgcttgaagaggtgtagcaggtttttgtttcaacttttatcactcttattcgtaaataagaattattaaaatgtaatgttaggtatttctgttgaacaaaacggttttataacatacagaagcaattaatgcattgaaatagtcttatagaaagcaaaacttcaacgaggaaacacattttggatgtttcagaaaaaacataccatcaacaactgtagagcttttcagaaagagtaaagttcctgcccagttttgattggccccgttatcaaaaaagtgaaacaaaaaccttgaaagcagcttgtttgttcgtttgtccctaatttatgttctttccttgctttcgatgatgcgatggcacgattttggcttgctttaatgatgcgttctgattaaggaccgattagacgttttttttcttccttttctcctcgctcgccagcttcctctagattcgcagagcatcggtgcgagacacaaccaacgttagcgttgataaataacaaactccaagggggttgttgttgttatgcgttccttttttgccacaatctccaaatgatagcgtaaacctgcaactatggcacatcataacgtcccgcttgagagagaaaataggcaaattaaaatgcgaatgggccatttttgctttcgttcattctgctaccgatcggtacgattttagtgttcacacacacacacacacttcttgatgatcgcttcattcatcggggcaacagaggggtggccggaatggtgttataacgtataatttgtgctaatggttatggggtggctttatttatcattaccctaacaaattgatagattccgttgactggctcacactttgctgcggccctgtgagacctttgctttgatcagtcggcggcagtgtgttctgggtgcgataggttccagttgttgcctccacaaaccgatcattcgtcgatcgttgatcgcgcatcccaggtacataactcatccaattgcgaagccccagcgtgtggtgatgaaggaagtggcgcagtcgccgctgttacgacctcttctgctagcatcgggccacggcaccgggtggcactgggggctcaacgacgtttgcctcatccggtgtccggctgtttggctgccaaacccgcgagcaaacataagcagacaaacaaaacgcgcaccgctcggtccccctcccagccaggccaggttcacacacaataagccggcaccgcgcgtgcggccgaatgccgcaactgttgaatgcatgtcgtaaaataaaaatttatgattgtaattatcatctcttctctcgcacccaccggctccgagcgaggatgggagggatgtggcgaacgcggcaccgagctggagcaaatcttcgcacacccgtctgcatcccattttcttcggatctcaccacatctctcgagcgctggtgcaaccggagatttaaagacaaaaggcaaaccatacacagacacacaggaaaaggaaatcagttcgcttggggtagctctttttcgcggtttgcagcacaatgataatgggttatgtatgtgcttgtgttagccctgttcttgctcccacctttctctagccgtaacgccacaatgccagtaagcttaacttatcccccggttgctgtctgtgttggatttattaccggtggcaagtaagttgcagcccattgctgcggtgcgcgcggtgcgttatggcaatgatttcgcatcttttcatcaagtggtgtgagcggcgggccgtcttggacacgcagaaaaggtcttatcttgtgactggccgtgtgtatgtgtgtggttctgcgcttaaagatataatttgtggcacgctttatcgcgacccgtacgacattgtttcagcagcgttgcagcagcacgcgccccatcggaaagaacggcttgatggacggcaggcgaggtaaataaaagatataaacgccgcccgccatgtccagtttaatcagctgtgtcctctggaacagttttccggtggtttggatgaggttgcatcgttactaagtgcattggtgttacgcatgcgcgaagaacaattccgtgaccttgtcgtgcgcaagcattcaaaagcgagaaaagcagctttctgttcagttagctgatgatttcttgaaacgctttcttctttttgacgggttctttctcttggaagatggtgaaccttatttttcattggtgttattagatgtcatgtaaccatgaagtacattcttgcctaagatattacgtcattcgtaaatatttattagacattgtagaacttctgctcagatgatttattcacgcaacacggaaatttacaaatcttttccacacttgttaaagtgcttgagtagttaagtgaaagagaacaaataaaacccagctgtggagcacaacagcccaaacgaacagggcatcctttagacatcattatgggtcggttctgcagggctgtctgcaatcataatgatcggttggaggttggagctccaaaacgcaatcagtccatacgcgcggtgcaagacgtgtgtcccggtgctggtgaggtaaagccattccggccgactatcagtcaacgcagcaagcagacaggacgaggggacacgctggatggatgcctccagagtgtgatgttctttggtggggtcggcgggtatgttgtggtagcatcaaatcgagcaaatcgagatggataattttcgattattaccgggtaccgaggcaaaccgagggaaatgatattgttttctcgagttgtacgtttttattcgccgtgttttatttttcgccatccctcctggtacccgttgctgtcaccgtcctttcaaaactggaaggacccaccaaagtcgtcggtaagcattcacatgcagccaggctcgcttgcatctttccgctatatcaacctggtaattgcatagtgtgagtatggtggtggtgctggtggtggtggccaagccaaagggaaaggggaggaaatacggagaaaagcaggaacaccaacatccaaatgcgctttgcgcttgcaggcatttcgcgcagcattaagcgaagccgacagaccacggccagcctgtgcacggatcgcacggattgggcacgggaagggcacggggagaagagacatgattgcttcacgccaccacgggctctcggtccgtgtaccagacgccccggacgtatcggaatgcgggctctgggcgtggctcacccggggaaaagctgataactttatgatgtgtcgaagatgagaaaatcatgactgttgtatttttatgtgtttttaaataatacaattgacgttatgttaacgggcggttaggctgccggttggaggaaaacgaataatcgagtacagtccccctgtacacgcagcacagggcaaatgcgaatgtggctttggagcgaatatgcggttgcggtttgcacattgttgtttggtttggtgaattagttcggcttcaaggtctggcttttgtttaagttaatgtcgtattttgagagtttgcatgatagtttttgcatcctgttaagaaccttcgcccgccgatgtcaattaataatggcagctttaaaaatgtgctgcacgttagctcaatcatgctatttgttgtgcgtgtgtgtgcttggcgcgttgcagaatgtatttgcggtaactagagtacaatgctgcatctgcactgacctagtcgtagagctgcccttctccaggccttgcgcacacatgctataacacctacaccactgagtaccaactgagcgcttctttataaatgggaagtcatttcgattcattgattgaatggatgagtgacgtgaaataattgcattcattgcagctctcgcagtagcaatctgcgccaccaggaaccgaccgggtgggacctagctcaatggctcaatgtcatcacagttgcgtgaatatcaaattgcacacggtttcccttccagatatatattcctataacaacacggtgccccgcggtccttttacggaggcacgatgtacgcaaactgctcgtttgggcagttccaaaaatacgcatttttcgacgcaatgacgatataatccaaagtttgttgggagcgcacggggtgaaaggcgatttgagtattctactgcaccgtagcgtttcgttttgtagccaattttccagtcgatactggcgcaacaaacgcaacggcatcaaagcgcgtgtcttgtacccacttattttctacgtcaatacgtgctgcgaatccgttgtcaaaaacacgcgtactactacgcctccaaaggatctgcttaaggaacggcttccgtgcgaagtcggcactgcttcttggatggtttctttcgaggcaaaggctctggttctggcatgggggtcgaaggtggttgaagaaagttgcacggctatttgtttcaaacatgccctagatagaagagaggctctggaagttctcgaagaagtatgcttatgcagatgttttacctttttttcgttccattgctacctgtcttaaacagctaccaatagtgcaccaatagtgctttggtgcatacgagaacgtttttaaacgtgcactgacggggataactgatggagatataaccaggctcaaggatcaaaaacaacttgatagtccagagtttagcgtattgtagcagaatcttgaagcatattgccaatcaactctgtacttgcgctctgagaagatgacctggtgatggacaagaactctttctttttctctttcgcaactcacattcactcataatttgcttcacaaaagaatatggaattgatctgttttgattgagtgtattcatatctttcctaatttcaatctactgactctcatctgttgctttataacggaagcggaagaaaatgatcgattcttctagcattaaacgagcatcggcatatcggtccagagaaacgccaaagacaaaagacgaaaacagacacaaacaacactcaaaacgaccggggaagtacgatcgacaaggggcgaagatacgggatacggtgtacgacgagttcccaacatcattatcatcattactgaagtgatcgcgtcatttatgatctgctaaagttatgaccaaggcgatcgaaagcaaaaaaaaacgaaaaatccggtggtttgggcgtagccgtgctcccgaacgacctcgagaaatgcataaattggacgatgtccaaactcacgagcagatcactgggggccatctcacggtgtgctcgataccggtgttccctgtccgaagcgaagacacgggcgaaagggaaagcacaagctgccggtagataatgaagctgaacaggcaatgggggccgatgaagagctcgcgtaccgaagagattgcaactaaggaaaacaattctgaagattgatcgtgtgacgaacacaacttggggcgctcactcgtacggaagagcaaaaaaaaaacggttaggcgaagcgaacgaaactatgaaggtaccacttgaggccactcggtggtgcatcagtccctccttcccctcggggcgaagggaaccatttggatggcggctggagaggaccgtttcaaatcgccacaaatcgatcaacgactgtcgaagaatcgtcgcgtcgtgtggacggaggtacaggggtggtgtgtgtggtgtatggtacgaccattgtctcacctgagcgcagcagctcagctcagttggctgttgttcggggtgttgccagccgctgcagaggcaactgtaggcgcactgtctggcggcggtacaggcagcttctttaaaaattgatttcaaccgcgaattgcggctcgagggggccgctggcgagccggcgatgcgcaaaacaaaggctcactgagagggatccaataaaatcgacaaatgaacgatctttctctcggctcgtgggttttttgttgttgtggttgatgttgtagtgccttctttagcaatcttcgtgtgaaggctgttcgcttaagtcacggcgatggtcaatgatgcactgcacactcaaccgtaatcatcttcgtcatcgtttcgccctccacagaacggaacgggtccttcccaagaggggggataggaccggtagtggcagtgcatccactattaatgcagaatcaatcaacggtgggggtcgagatcgaaacacacggctatcgcgtctggattgggtgcgatcgggccgataggccggctctagggaccgctggctacatcgtcctattgagctgtctggatgcattgtgtgaattatataattaatttcctttgcgccctcccaccggtcgagcgtcactgagagcagcgtgtgtgaacgatccttggtgcatcgcacgattatgactattgtcctcgggcgagaacaagggtgtgctgcgcctggatctaccttgggcgtgaaggaggaggttcttatgtgtgtgctaatctgtcggtcgaatatttgccacaatagtcggcaacagcagcagcagtagcagccgtgacgaataggcgcctgacggggtgcttttggtgtcgctttttgcgagtcagttgttttgcctcatcattctcaatgtctcaatggcttcgatgcggccaacatcaaaagggtttgatggcagcatcttcacagcgtcttcgtttactgcattcggattgaaggtgacctattttttaattatttatggtatttcatccaaatgtgatttttgaagctgattcttgtttgtgttctttgtgtatctgcatggatgttttgtgcggatggatgtgtttgatgtgttgaaattatttcacatttattgctgtaacctttcaccgttcaccgtgacgattgcatatctttttttgtgcaaataatgtatccgtaatatcaaaaacattattagaaaaagaagtgttgtaaggaaacatactaaccaatagctttgaattagtctgagaaataaaatagtctaaaaataaaaataaaatattgcacaaacaatttgtatagctataggcttagtctgtccttgctttaaagactaccccaagggttgatattcgtagcataaattatgtatgagagttattgattgacttaaaatcgctcacctgcctgtggccgtggctgtggtagtatcgaccgcagccaacatgcaatgtcccaggtgtaacgacacaattgcatacaatatagaagaaccagacactggctggccggctcgggactgcaaatgaaaggcaaaatcgaataacgaagaatccttctaatttcaacccccgtcctgttcctcgtggccccgtggggtcatggggtgacagctgtgtgtaaacctcccggagaaaagtaaggaaaaacgagtgagtgagaaaaaaaaagaaaaaacaatcccaggaaaaaaataaaatccccgtcaaacgatggtgtccgttgttgctgttgcagaaggttcgaaaaatagacaccagagcgtttattgcctgccggtggctttgcaaatggataggattaagtgttgtgcaggttagccgtatgcaactgattcgtactgaatcgatttacagtggagcagcagcagcagcagtaccaaacaggcaagaccattcctgctagatacaccctgttgctgcagtttcgaggccaggcttgacgctagctatctctcgctgtaagctgtcgggctgttaaacgctcgtgttaccgtttgcgatgcattaattaacgaagtgagggcgagcagacggctgacggggcagggaccggcaatagcggagctgtgaaaatcattgacattggtaaatttgcatatattgttcgcgataaaagaaatgattaagaaatgtggagtgggccgggtggccggtttgggtggctgttacgataagcgtttaacgtcgcattaattagtcagagggtatccgagcccaagtcgatcatttcgtgctgccctggtcacggttatgatgcggtttgacgttcaactgtttgaagacgacgcgcgttgtgactttcgctgataacgccgtcttaatcgtgctcaatcacatcgcaaaactgccgcggtgtatgtgcgtttctaagcggtgcaacggtgggtggcattgaattcctcccaggcccaggcattgtgacgcgcactgcacactaatcttatcgcctttgatacacgggtgtcctctattctggtcactcgccactccgggggtagcctttcagtttttgccaacccgcttcaattcctccggtctcaacaccctcccttgcacatagacgtgcttgttcattagtgttcctcttcaccctggtggtgccatgaacgcacaactcttccgcaagcgcatcgtcgtctgtggatgagtgtgggttgtgtggtttacattgtactcatggtgtttgagtttgctttttttgttcttcctttgcttgcgttgtgcaatactgctacgaatgtcagatttctagtcgtactcgattttggccgcaaacacacatacgcgctgctctaacgccatggtctggtaggtccgagtgcaattgtgttatcagctggcgatttttgccctgcattttctttgccgcgagtgacctcgacttgggatttgctatgtaaacataacgtgtacgtgtagctcgtgcctggaatagattgcctccccatacagccagtgacacgcacacacacacacacacacagacgcgtggcacggctgtgtttatgttgcaaagattagtttgtgttggtgcagtccccgttcgctcaaagcaatgcaaagcagcagcagcgacggcaccccggaacacattggctggtgactttggttttgtgccccgtccccgtgcatgccacccggaaatctagccgccaacggtgactaggtgtattgatgaatttaaattttgcactacaaaaatgcgctttgctttttaaatggtacatgtgcaggcgactggttgctctcctttccttcattgctgcattgccgctttttcccaatcacatgctggatttggttgtcttacccctccctcgcacacacacgctcgctcgctgcatcactaaagagcatgcgaaataacgataagtgacagttgaatgttcagctgtttgctgctacccggggtttcgtaaagccatcttccaccgtgcccgacccttgttggcgataaacgcgcgctcgcgaaaaataaaatcaaatacgccaactggaagagcagttcggctgtacaacacaacacacacacactcacaaacctagccgcactaaacagagcgcagacagcgacggcgacaagcggccaaagacgacaactaccctatcccaaccccgcgactgacaagtctcgggctcttgcgttccgcttctaattaagcgcggaggcccaccttcagcgtacagcgacgacggtggcagtccttcgtactcgtttttttccttcctgtgctgtgccctactatgtggtagcactatgtggcactgttgcgaaggagcagtatagcaaccacccacgccaacaccccaccgggccgacgggagctaaaagtctgacaagttcaggcagctcgcacgggagtcgggaatcgattgtatcgatagcagcccaagcgtccccaataatcgacgttaaattgtttcccccgttcgcgttggattgttaccatttgcgtagttacactgcttaatttttaggcgtaatagtaccgcatcacagtgtcgtaaactatcggtacgttttgacatgcagcgcgttgaaacggcacaggcaggagagcagccaaaacgaacgggaacgcataaaattgggttagctgcggtggaggcgtcacggtaacgagctggaagctggcgtaaagcgtagatgaagctgcacagacagacagaccacgtccacacgaacggactgggaagcgggagaatgcacgttgcaatctttgaatctgatttgcacgcagatcgatgcaaaaatgttgcatgtcaagcgttaataaagattggtgtttacgagtgttcgttttggctgacaccggccggcagcgggtgaaacatgcgacatcatacctggcggtacttggagcggagagttggagctgtgccagcaaaggtgtcaaacgtgcagcttatcgaaagggtaatgaggcatttacttgctctgtcgcaagacaattactcaagaatagaataaatacaacaaccaaaaaagcccgcaccaatttgtaaggattcattccagctctcccctcgcagggtaatgtgtgtaacaatacgaagtgtgacagacacttcgggggaagtttttgacagctcctgggaatggcaacccttgcggctgcactgctgcacactcgacaggggttttacacgtgcatgcgcgactggtcactccgtagcacacggtaaacaatgttgtaactgcaactcgccccttaagaatcctttcgcccctcaatttgtaggcaagtttccgtctctttgcacacacgctgaaggaacagaacgtcgtcctatgattatgctgtcagggagaggaagaaacagtacgcagagccacgccggggcacaattcattcgatcgggaccgggaggaaaagcgtcctcgtgcacatttgcacctcaatagcgagcataatttagtcaaattaagcgtactccgctgggagtggacgacgtaggtcgtcggtggtggcattgtccgagaggactggtgccacggttgctcaattgtaacaatcgttgacctaggtcggtggtgatgtgtgtggccattgtttcaacattccactagcttcgggtcctcctaaaatccactccccggacggatagggcgaacgcaagtcacgggcagcgactgctctgtggcgaggtgtttgtgtgttgcaaacttttgaaccgaaaactgctacgaccaccactacttcgctgctgttttgaaccaggagctctgcatctcctcgactaactgacaaaaaagaccgcatccgctcacattgtttctatttctgcagggacagagaggtggtctagtggtgccaaagttgcccacggtggccgaattcgaggccctacatcctccaactaatagcagtgccagcgcctgctagatcctgctactagcacaagtgtgtgtgtgtgtgtgggtgggaagttcaatgttgaaatgtttcaccgatatttatcccgacactgaccccttggatgagccagcgttttggtgccatttctggctgtgttttcgctcaaaccaaccagttcgacaataaccagtgatgttgatatattcacgtgtgtgtgtgtatgtgaactttatttttctcgcgttttcccgctggaatgtgcatgacatgtcgccgcaactgtcgacacagattcgctctagtggaagtgcatcgtcgcgcattcgctgctgcgcgggctatcgcgggtatctagacatacgtgtgtggctagtgtaggccagggagtaccatcaccacaggaaggaagtggttcgagagggcgaatgcgcgccacggcgttccaaaacacaaaaagcggtttggatccaaactttactgcatgttttccaccggcagtcctgcagacgatggatccacatggacactggagggaacagcacagggtcagcgtcagcagtaactggtcaacgctgcgttgcgttctaatgtggggcttccgcttgtctagagccttccgcggagtgagtgtgtgtgtgtgtctggctgtcctgaaaattggattcagagcggatgttgactgtttcgcgtgtgtgtgtgtgtgtttgtccagccgtggattgttgggagaatatgtgctcatccatccatgcggcaagtcgctcacggggtggaggtcgcagcaccgagagtttgtttggcattaagtaccttcagttgcaaaggcaatgcaaagaagaatcatttatcaaacctaaccatcttcgctcaagggtttgatattaccctcggagaaccactttgactcatgatccggcgttgagcatttttctagtttcacacattgcagtaattgtcattagcacttaagattgaaagcccggaatgctttacggcattggcccgtagatcgcagaaaggccgcgagcaaaccaaagaaatggatgtctttatcgcaacgaaacgtcgcaaattttgcgcccttttttactgccccgcaatagacacttgcaacaagacggcagcgaaagagtaaaaaagccagagaaggcattccgccaatgctgtaaaaagcaccaacaacaacaacaccaacaaaaaaaaactcgaaccaaacgcacactcatcagtaacgcgagaccagtgcgaccaggcacccatctcccttcgaacgcgcggctactttcccagccataaatcatccacttcaaccagattgagtctcctgccgccgcaccaggcgtgaccacacgtctggtgcggtgtctcgtttgttccgccgtttttgttggcgtgtgggtggtggtggtgggggcgggggagaaggtaaattaatttacacttgcacacagcgcagcttcaagtgggagatgcacttgtcgtctcattgcctcgttgctgctccggcctgcattgcccgccgtgccaatgacgcagtggggttttggtgacgatcgctacctttaccgcgcttgatataagggttgaaaatcatcatcatcatcatcatcatcatcggatgctgatcggacgggccacactcttgacggatcgtctccatctcgttgccggtccgctttcgcctagccccctcgtcgccttgcccgttagcagttcgtgaagaaaatgtgcataaaattagaaatcgaaccctccgcacacaccccaggagggaggggcggtatgattgggtcccgtgtatgggtgtgatggtgtggggctcgatgtgagtggcaatacatttgcaatattagtggttagattccatttcctgcacagggagcagcgcagcggaatgtagaaaaacaaaacgccggcaagaagtgcggatgcaaacttgcaattgttggttctgcagctcgggtgcgggtgtgtgtgagtgtgtctgtttgttttctttgcacgctgcctggtggccccagggaaggagagggcgttgttatgggagaatgtaaaagcaaaacaagccacccatccccgttctattgcatctcgtctcgtggtccaagaccactccctatccctctcgcctcttcccgcccttaatgtccctctgtaaagaaagacgatttgttctcacattcctgcttcctccttccccatgtaccaccatctctgtctggagaatcgtgcgcacacacacacacagccacaggattgtgacagtaccgtcccctgctgggaggtgagtgaaaagaaacacatttcacgcgtgtgtgtaccctgtgtaatgtcacagtcgatcacactcgggcccccgggtgaagccgattgaatcataaattgcacttacggaagcacttgttcgcactggcctgtccggtggccacaaccgggtccgagcggtgtccatgtgtgccgcattttattttgcagccacttttacaactgtgctgctctgctcccgctcccgctgcaccgccagttcgagagatccgagcgtacgagaagtgatgatgcaatcaaccggacgggaggcaacccatcgttagctcgccgctggagccgatagagccaacggggccgggagggaaggatggaatgtgtaacgctgcagctaaatggcgcgtgcaccaacaccagctcgcagcggcgagaaaggcgtaaattgtgcggcgcgtgtatgattcttggccggggcgcgttctccctttcccccactgccaatcgttctgcccttctggatctgggcgggcggcatgtgactagctaattttccaactcagtggctggccggcggtccgtaagatgatcacaatcactttggaacagtaatgtgggcacaaactttcgttggaaggttgagttttttttaaataaataaaattgttaaatttccaccaccaatttcccccgttttcactgttccctagtttgagtttgaaggtcaatcaagaggaaaagaagaagcgaattccctgcgcaatcacccttcgcgagagtcggaggaagggacgcgcaaagaatcctattgatagaagctactgcagctactacactacacttgcgtaattgtttaacgtgcagaatgaatcggtgcactatgcggccgggaagtggccgtgtggtggggcagctctcccccgttcccgcggcattgggttaccagcgtgagcgtgagcgcgcgcgcgcgcgcgaagaatcgatgatgccgtggaggttgtcgcgcggcgcaaacattgtggtgtgtggtgtggcctgagaccggctgctaggggaagataaaatgtagctcgggtttgggtggcggcgcgtgctggtttcgtgatcgcggctcaccttcccaatcggatgggcggcggttgatggtcgggcggggagtagtatctggtgttcattgctgcagttcggggcagaatctgaaggcccaagcatgggcgaggcaagtgacgcaggcgggtgccgatgcaccggtaagaagggcgcgcgaggcaagctgataagaatgtgccggctgcacaggctgcagttttcggtctttgtctttgtcgcacggcattctggagcaaaagaagaagaagaaaatgatgaaaaagaagaaagatgcgtgtgttggatgattgtagccgaggaccgatgcgatggtgcggttggtggtgttattggtcagctaatggtgagccggtttgccactgtaaaaggtaatcgcgactcgaatcgtcgcgagactaaatatagagcacttcctgagttcatgccaagtggcggaaaatggacggaactgcatcgcttgcccctcccgtaccctccttcccctttccaccagccacacacatgcacacttataccaacacagtggggttgaacagtgcattggacaaaatgcacgtgtaaaaaatgcaacagcccatgaatgtagttgtgtgatatggtgcactcattgtgtacgtgtggtttttttttacaaattacagtgtgtgtgtttgtgtgtgtttgtataaaaaacactacttacacaaacgcgtttactcgtgaagatcaattcattgcaacgcgccgaatgactcgcgacgattgtgccgtttgggtggatgatgaaaagtaaataacattctttgggtaaatagttgcaacccgaagctagtgccaactgtgctggcttgctcctttgctggcgtgttcgggcctcgcgtctcgtctcccgttacacggacacgtaaatggtagatgtaaaaataaagtttcgcgtcggggttgtattgaacggccgtctggggtggggttttgaggggggaacgcgggtatggccaggataaaaggtgggtgtgtgtgagagctccgaggtgaacaatcggtcgtgaccacggccgggtgttgtgcagccaggctgtgtgcaaactgcagcgagatgcaggaaaggggtaaccgttttcggcgagccttcttgtagtttcagcaccctcggttacccacttctcctctcctagcttcaccacacgtctgttgttgcgggcgttctgttcttctttcactgatgtttaaacgtttcttgaacgatgcgttttgcgtacgatttttgagtttataacacgtggttttgcgacatgttaacatttacattgtaatcagttgattgatgttaatcttttttatttatttgctctccttttcagctactcactcgtgcgtttcgccagaacctgtaaatctcctacctggtaagtaaatataattaaaaaaaggaaataatatatttcaaagcggtacaacggtgttgtagcaaacatttagtgcttcacactgtacgtttgaatatttgctaacacgatatgttacagccgacattaaagcatcttaaaccaactgaacccaacatgtagttctttgcaagcaaataggacgtcatttgaaaaatgtgcatttatagctcatactttatggaatgatgtatgttcttgcccgatgcaatctgctatagaccacattgcaggctgcatgttataaatatcggctaacacaatgcgtcacctttttctcaccttaccgcgctcggacgcttaaatcttgtgggcgtttgctttctttgaccttatccttgtgcgctaggctaagcgtatttctaagccagtggacatgaggtactaccggcttccctttttcgatatgtaacacagttaacatcacaagcacacacacacacacacacagaaataatgtcggtatggcaattggacaatattgttatttatcgccacattcaccaaccgatcgaaattgtcccaaatcgcttcgagtacataattctcctatctgtctgccgctggtggcatttgtacgaaaacgtataaaatgccccgttcttaaggcgaccgccacacaattgtgggcattgagctgaggggcgcgcgagactcatgtttgtcgcatgcacatcgcggcggcggcggtgggagcagcggcttttcgcgcacctttgtcgccctgttaagcatttttctagacgacagataccagcgcaaatactgttgcattatacaccgggtgtttaagcagggacccggtggtggacataagcagaacgataaaatatttgcaaaaccgatgtttctttgcgctgatactcggcggatacgagcgctgtgtttgtacaaaggtacaaacaccgagagcgtgtccgccatgggaaactgcctcaaacatacgcccttccgtccccctcgcctcgccttttaccaccgaaagggcaaaaaagggtgttaatcgtttcgctgtgcgatgtgatgattggagatcacgaagatcaaacgggtgctggggtgaaaagcacgatgctacttttgcgacataatgcgctcgcttcgatgtgttgcgcgtggacatgttcggcatgcattcttcgcattaaatgcaatacgcgattattttgaaatgaaaattgatcgcaaagaaaatctcaaacgcttgattttacttccaaaaagaaaggagtgcgcaatgcgaatacgagagtgaaaaagagagcgttatgacagtgcgcttgatggctaatttgcaaacaatttacataggccgcatcagaacagttcattacggatcaaaataaacaatttactttttgctcgtatttgctttttttgttgctccccgggcggttgttgcgatgacccgtcaaaggggatcagcggtaacagcggcgaattcggcgcgctctcgtggccgtatggagataaggcgagcgtaaagagtgcgaaggggaggaagggacctcgaacaagaacacgactacaatcgcacagtacgaaaacaggaagaaactcggaggccgatgtaaaactggccgcccagggtctggacaaaactctttatccaagcaagcactgggaatgggggaggaacaagggcgctcctttcctcggggccttgctggctggtgggcggcagggaccgggggaaataacaccaattcatgtcaatgtcactgtcactcaaccccaacatgcaactgcatcatgggggcacgcgcgaggttccctcgttctcctccgggaagttggtttccttttttaatcggtggagtgtcgagaaggggtgcaggcacgaggtttgggtaggtacagtgatgtagggggagaacgatgcgtgtgcagtgcaatgatcaaatgatacaggcaaggagagcgaagaggtcacgaatggtggaagtacttgattttcaggaatcaatattcctcgctgtctgtcaaccgttctgtccccaaaagctggcggtggggggatccggtggatcacgatgggtgagaaaatgagtgaataaaacaaaaaacccgattgcaatactaataataaaataaaataaatctcctgcctcgtccagcttttttgattgtgagcctgatttttctctacattgtagccgatcgtgtgcgggggatgtcagcctggggcagatggcgcaaaagggttgccgtacgcaggacaagcagaaaatcgtggcttgaagcccgcacaatctatttcctttggttgttttaaaaatgggttgcatccagcttagtctgagctggaagttgtctcacccgtaggggcaacagggaacacgaacaggagactcgtttccgcatcggctagcttcggtggaaattgaaggcattcaccccttttttctttttctagtccataattgcgggtgaaaataatgccgcagttttcgtgccgtccaggggacaggttttcttcctacaacatgattaacattgcaacatttgttgtaacaatgcgattgtgtgtcccagtgcgtaaaacgcacgagcctccgatcatgatgggcatgggaaggaaaaaccgttcgacggtacatttgttgcgttcgatcattgtcaactccattaaacgaacctgaataaaccggtgcgtgtgtgtctgcggtgatggcgatctttctttatcaaacaaacgtgtttgagtgttctggaggcgtttgagtgagcagcggccatttgcattcacgaagccgagttgcatcccaataaaaccaactgcatgagatgattgatgttgggagatgagctgcaatacattcccaaccgtcccgtttggtgtttgattgatttttcttgcaccgagctgctgcaaaccgggcccctggatgcgcactgatttgtttgcttgctggttgcaacaaagccacaccaccgttaaacctggtgatggtgatgcacctgtggcggatcgttgcgatggagcgactgatggtgtgagctttgtaaatggaatttcacgcgtagcgcgtctagacaaaccccaattgcggctgcagccccgtcatgcgggcacgaccgaccggacggccgagaccggtaagacagtgttaagtggaaatgagctgcggaatggctggcatggtcgtcgtggcaaataacgttggccatgttagggacacaagaagatgccggtatttggcagaaggtgcaaacgcacacaaacctacgtgaatgcgatgtcttctgaaattaactgtatcgtttgatgacacaacgcaaaacgaaccagtttgtcgttactttgagagaagaggatcatgatgatgatgatgatggcggtggtggtggttcctcaagaaagatggagtgaagcaagtgttagatccggttaccgaagcgattttcaaacgcacagtaatgattagcgaacgggccccttactgtttgcctgttggtggtgcagtcttcaatcatggaacacgctgggctcataaggaaacatggggcataatggtcatgtgaataattttgctcttttgataaatcattaattatcttcaaaatcgttgaataataattcaacaaaaattggtgctttaactctagattcatggtacaacatgaactgcactcgtttacaaacaaaatcagtttaaaaaaatgtcagacaaaattgcaagttgcaaaattgccttaattatattttttataatgatgcgaagccaaatggtaatcggccgatcccgtcagatcagttgtcaatcacttacaccggtttcgagcccaagtaaattatgtaaagctgctttagaacgttgttcaactgtaagtaaacaattagcgtccaactgaaatacttatgcgtttctgaacattgttcatttgtaactaaacaattgactcctctaagctgatacatttgctcaatagagtttatcaatttgtttttgttttcacttacaacaataatgcgaatttagttgtcaataatgtgtatagattgctagaaaatttctcatttattataactcaagatcgaaaccaattaaaacaatttcaaaataatttaatttgaatagattcagaatcaaacaattctgatgcccgacgagctcgggtaatatagatgaatgtttatattggcgaaagcaaatgttttgctgcgatttgacaatgttcaaaagcaccttagcgttgtttagttgaaaactttcgaaaactttagttgaaaacgttggcttgaaaacaatataataacttgcccgtcataccttactttaaactctctttctttgagtaaataaacaaatcgttgatagtcaatccgatttatggttaacgcaaattgactttcgactatggtgtttgcgtcaaatgagaagaagataatcacaattatttctgtaactatagccaaatgataatggtaaaaagacaacaaagataataacaagtgtctcaagtgtctggatgtgtatcctttatttgataagactgttttctagactgttctaataattctacaagaggctttaaacatataaatttgtatatattgaccctatgatgattttgctccgagtgtccttattatttattaattaactatttatttatgatttattataacggacacaaatagaaaacagttatttttgcaagactgtgcatttttgatccgtaaaaacagttcctggaaaaaagtatgcaactcacagtacaggtgaaacataatacagcggttgtagagcgtactgtttggacaagttaattaaattgcacccaagcgtgtattaattgtacccgtgttcggcgtgacgggcacacacaggatcaaaccactactgagaaactggatctgcttcgttcgcactcggcggtggaaagtcctttccgcacagcacaggacagtgcagattttgaaacattaagctctcgcaaccggcgtaaccgaatccataaaaacggaggttcctcgtccgggatctcctttcttccaagtttgtgttgctatcttgggtcgtaaatcttaacagtagcagtagttggacagtgtatctaaaaaggtacggataccaaaaaggcacgagtagaaaggagcatgtctagatgatgctggtgctatcatttggctccaattcggacatccggattgacgtcggctcgcggtgtatgtgctttagtgaggcgattgtaggtagcaattctccctcgtgttgctcctttccggaatagaatgcaacaaggcacaatgttaatcactcatcagaaaagacgaaacgggtccgttccgcaccggcaattttccggctcggcacagtcgatttctgcagcccccgtggggacacataaacaagcgaccaaacaaacggaacacacattcttcattctcgttgcgctccactcgtcgttttgtaccgtgctggagctgtcataaagcatgtagtgcaaagaaagttctcatctgagcgcttcttaatgctcacacttgcggtcccgtctggccttcggcagctccggcagctttggggcaattgttgagccgtaggaggaaaagacacggtacatataacgcccgcctcccagtgtgttgagggcagctgcccgtgctactgtgctgcactgggattcggcaaaacaatttcctaaatgtggtcgaccgaagaacgaacaaggttagtgtgtaccttcgctgcatcgagaggtacgccacttctttgggaagcaagcaaccgctcagctcctggtccagactgccgaaactctcaagtacgtttcggagattccttcgggagcgtgtgggttgtatgtggcctcggttcaagaggtgggtatagcacattttatctgccgcactgccattcgtgatgcatacatcaaccgttgctggaagtaatcgtacggagatgatagacgagcgatgaaaaatcgcacagaacaaaaggccatgacacgaggacgaataaagagttgccagggcgccatcccaccgaggggatgccacagctgtctcgaggagcaagccgaaatgatttgcattcagctgcatcgtgcaagatatggaccggtgagcattggctgatggagatgaacgtccaccagagataccaccgaacgcactgtctggtggtgtgcgcaaggttctctgtgagtgcggtttgctgcgatcaaaagactgccgagagcctgtcggcttatttttcggctcggcacaacaggctttggggttgtaaaacaagcaacaaacaaatgtaaatatcgtgcacaacatcaggcactgtttgagtgtctggttaaataaagaaacggtccaaaatttacagtgcgatggtagtgaagtattgctttgagaatggtttgaaaataacggtttgtaagttatctatcaaatttgtcatcatgcacataacttacaagccaagttatatgtagttgattttagagatcaaatacgttcctccctgccaatgcaataaaaaaagccatccaaacttgagacatttgctgtgcagtgttgggaatcgatccaccatgttgtaatttcaacaataacaaaccgaacaatacgcctatacaccattttaaccgactttccccttcagggctcagtcccgcttcccactcttattggagcgtaagtgcagcaaacgtccaagcattcgctctgtagcaagcggtgcaatcaacgagaaattacaggcttccaggctaccaatacgatcatttcagctgccacctctctgccacctcgccgagtgtaggtaaaacgcatcgcctcgaagcatttcccttacgtcggagaaggctatgctccatggatgccgagttgccgtggatgcgcttgtgttgcgttgttctttatgaacgcgttgaaccttccacgttgaacacagctgaggcgagcttccagcgttggggcgagcctctttttttcaccgcctccccttttacccttcatcaacggcagggcgagtgcactagtgagcacttaattaaaattaaactaattaagaaagctcgtcgtataattttcacaccacaccatcattttcgggctactggtaatgaaattaatatttcattctattttattattaacgtttacatgggggggggggcggggggggggggggcagaactcggggcacagttgtttggtaaccatcgtaccattgcagctcgaccgtttcggagatgtgacccttgcaacagcgtttctttacttaccattagtgcgagattttcatacgcgcggggagctctgcaccacattaatctcagaactcggaactgctccccttcgtcctcggccaatgttaccaatgctgttgatcaagcgcagtagcacgccgccctcccagtagcacacgatcgcgcgtctattaagtgttcgcatgtgcagatcgctttagcagaacaatttatggtgccggctgtttgagaagcgggctgccggctacttacttccgcttcctccgatgattaccaggctggtagctggggtcccggtggtataagaaaaagtcgctcagtcacggacggcaacacatgaatgtttcattgaactcttttgccgggtgggcggtggctaaggctgaaagggtgcttcagcaccaaaactggaccggttcagaggtttcgtcgttttcccttagaacgtgtgtgtgtgtttgtgtgtgtttatccaagaggtgaggacgaaaactgctgcacgattcttcggcaccgagagattcttacccgggttggcctcgtagtagggtcgcaagagcaggccaagggtttgggtcaatttaaaaaacgggataaagtgtgcgaggatcaagctgaagctggtggtgtgtgtccacattgtttgatgatttatcttctgttgctgtttgcgattggagcgcgtgcaatcgaagccgtaatgctaataaagctggaacaagcaagaatctggatcaggcaggcaggcgggtgtcgggtgacacacaagtgcgccacattatgaattattcatcctcacgtgatggaagttaaacctctatcgtgctggtgcgagtacggcctgggtggagagtttacaaactcaaatgtcaagcgcatgtaaactgtagaaagtgtagatcgctacagaaatgtctctatttcatagtgtgaccttccattttgtagagcatgtcaaactttggaagggaaattgtgtacacggccacaatatctgccatacaactcaaatcaggctatagtttttttttccacaaactgctgatgtttaattatcgtgttctacccattgcttcacgtaacgttggaaaatgctttacacttgcaatccgcccattttcgggcgtttctacacactgattaatcatcgataccaacgctggtaggtgttaaaaggataaagccggtaacaattaatacagtttcacggcaagagcgcaatcaaggagggaaatgattctttcgctttccgttatagcctcggcaaggtgcatcgggagaaaatattgcatggtaataaattcccccctcccacagtaaacattgcatccaacttcgggactacagtgtaaaggagtgcatttttattcatttttttgataaatcactaaatgtgaatcgtactcatcgtggatgctttatgctgatggctaccgcttgccgaattaacctgcgaagactgtgataaaacgttgcttacggctcaatcgaggaaccggctacatacccactaactccacgcgaaggcttgacctctagagtgctttccgtgttcagcacaaccgaattgtacaaaagaatatggtaggcgggggacacaaaaacacgttggcaatgatttatcggttggcattgccttctacattgaagatacaattgatcggtcggtcgcgccggttcggtcaacctttctcttgcctcagtgcatcaagtgcagcgtaaatgcaacaatgccgcgcgtttcctcgtgcccccggccttgcgggtaaagtacaaatgcagtttatttccaaattaattagatccgctgctaaacaatgttctcctcgagcaaaaaagcctaatgagatcttcggccgcacgaaatttgtgccgagaccgcggaccctacaatggcgctgcaaattaccgctttttccgttccctttttgtttgacccttgcgacgtcctcccctcacgccgatcaacctgacgggttcctgatgggaggcgcagagacagtggagtgacagttatcgacacttgcacggtgagcaaacgcagggaggaggtcgctggtcattagtgggttttgggctggagatgggacggcgtcacacactccacggaggagaggcagcatagtgatgttcattttggactacaattcagacagtcgttcgcggtcggacagaaaaagtgctaatcgaacgcattgcatccagcgtggccgcgaacttgtgtcccggggcagtttgggtcgcgcattggaaagttaggagtaatggagtgataagggtgagtgtggacaaggatgatgatgttgcttcgggtatgagtgcgcgagttgcaaagtggcaaaaccaaatattgtaccgccaagggatgcatttggtgcgatgcaccaaatcgagctgtggttgcctctacaagaacctgcgcgctgccattagcgcctataaacacaacaaggtgtgaatgttcgaattgggaggtgagttagcagtgtgacaaattgatttgaaatgactgtttaacataccaatacggcatgggcaatacgtactgattacaacaagtttaatgagttaaacaatatacttaatttgttgcattcaatcctcagctaacaattaaaagttttttttgtgtgacgaaacaacaacccatcttaacaaacaatatttcactagccaactagaagaataaaacaaaaaaacaatgcgaatgaaagctagatactactaacacagttcaactgtttgggtatggtcccgtagtaaagtcgatataacggacgaaataacaaaatgttccatccaggtgtaggcgccataagacacaatggtacatcaatccattgctgatgattaaaccctctagttgcttaggcatgtcttgatcaactacgcttgttaatccaaagaacaagaagaaaaagtgttaatccaaagaacaagaagaacaagtggttaattcaagatgtatcgctcaaaaaaaccaactgagttgactgcagtacaggaaaacaaaatcttacagcttgaatatttttattattattattattattactattacaccatttagcagctgttgaaaatgtatgaaaaaatgtgtacaaacactgtgtcaaacataattccaacgtgtcatcaattcgcgacatagctgtcccgcaaatggcagtaaaaccccttgaaacggtttttaaatccatcaattaaaaacgagcccttccccaacagaagaaacagagagacaatcaaaaacaatatgcaaaaaaaagatgacggaaagcaaaaattttatcaaaaaagaaaaaaaaatgcaacagaaaaacactcccatgggggtaaaaaaaggaaacaaaacatgcacattgtacgaaaacgtgttattctcttccaccttaccattgcgtgaacgatatgttatgccaaaccgctcgaggccgatgggtaggcggccgtgtgtacgtatgagtgagttaccaccaccatacctgtcggcggatgttcaatttcgattctgtgaatggatttacttccgggtggaattgcaccgtttgaaccgtttgaactaccccagaatgccggggcggttttgtttttctttccgttccgaacgccgtatggaaaggaaatggattgttgttagcacgtagcgcaagccaaaaaaagcaaaaagagttggaaagaatgaaggcatgaaacgaagagcacagaacagcagtagcagcaaatacgattcggcaaagtaaatttacatattcgacgatcgacggctggttttcctctgcccagcgatttgctatccattgccgcggtgtttggcgtggggaaacagcatcggcacaaggaaattggccacccatggggggagggtactgcttcgcttgtccatcgtaatcggtgcccatttgcactcactggtacatggccaacacagagagggagagagaccggggtggcattatttgggggagttggtgtcggagcgtgcacttgccaagggtgtcatcatgtgccttgaacgttgcatttccgattccccagaatggctgcgatacggcgagcaagaatggttagcgtgaaacaaaacagtcgtttgatgattttgattccgtttcgatcggaagagttggtgtgcgatattgaatgtgtgggacgggggtggcgaacgtttttgttccctgtacagatggactgtcacaaatttatgcaaaatgtattaaaggatgacgtttcgagtgatggagccagttcgtgttgttttttcgcgcaagctctaccattttcggtggtcgaatttttgcgccacgtttactaaatcgccaaacaacgcgatccaaaaatgtgtcagctctctttgttttgattttggctggcgttggaggtaaaaccaacaagaaaaaagaaaacttaaatcaaataaataaaacctcttggccggcactggcgggagaacgggccacggctagctctgctaaattaaacactttgttatgttttgctgcaacttattatattataagcactgctcggccgacaggaaacgtattgaaatttacgattgcaacaatgtagagctgttcgtttgcagcaccccatttgtgaatggcacttgtgcgctggaagtacaaatttgaatgtttacagtctaagctgtgcgcacaagaattgtcacccgcgaagaaacaatcatttcgacactttacccccggttcccttttcttcggctttctctctctcccttgccgctgctggttcgtcgctggttcggttcccacagctgcaaaccatttaaacacttacgcaaaacgcgcgttccacttccagggcaccgggaacaacgcccagaacgaaatatcgttaatctccttcgggcgtgtccttgcctcgcgggtacttgtctcttggtttgcccagcgagatctgtacggccgcgtgtacacaggctcttacaatgttgcgtgtgtgtgcggagaaaatgtgtaatcgatttagtggcgcaacactatgcgcaacgtttttctattaatgcacgtctgtgcgttttgtcctgcccgaagacgcccaagacactcttcccaaggaatgtgtgtgcacaggaagtgtcaactcgtcaaaccaaacgcggtggagtgtgtgtgtaaggtgtcgtaaatgtcatgccagcaaggatagggtatttgttgttcttaaaatttacgattacccgttctacgctagtgcgcaattcgttttgggcatgtgcttgttggacatgttgtggcgggcagtatatgcaaagcaaacagagagcataattgttatgatgactgcgctcctttcacggacggagcggtttcagctggaagggcccacaacactcccagctcagaagcaaaacaatttaatgacgaatcgtggaaaaagaaaccaattaatggaaataaatactttgttgcgagcagtagagggctgtttagaaattttggtaactagcgattgcgtgtgtttacaatgtattaaaatgtttataagccgtataactatcgagcaggaagcattgattctttcaaacaaagattcggattcaatgtcgcgtcgttggatgaacgaacaatattcttcaaattctagacagcaacaaaatcgcgctgcaatacaactataccgttgatcggcgttaaaaagtatgcagacacaaagtaaggcaacaataattacattaattcatcagcgaagaacataatcaagcatagctggagtgttacactggttacatgccaatcggtagaattcattaggaattggtcggcaacatcgtacctccggcagaagaagcatactttgtgctgaccaatgcaattcgttaggcgagcagtctccctttgatgttttagcatcgatgaagtgatcaatacactgaccatgtgtcggatttgtgtgtgtatgtatgtagtctggcatgctctctctcctgtctagcgaaaatttcaaatatcagtcaaatgtgttccagcagcacattatcgggacccgtctagctagtctccacactcacactttccatatttttcacaccttggtctgaatttgtagtcgtccccgtgcgggcatggaaaattactgtgcaactccggacggtaggtgttgatgtatgcatccaataaacacttcacgtgttttgccaggtttcgcgtactgcaaacacgggctttggcgtgccgtacgcgtacggctgacaagcgcgtgcgacaaatgttaactcgccacctcaatcaacaccgtagcgtaggacggcgaacggtaggcgcactccgccgggattgacatgaaatttcgaacgtggttcgaacaatcgacctcacccttacccaatgatttcgcgccgagcgttcgaacgggctaattttcagaagggaaatcggcaaatggatggatgtgtttttccggccgtattatgacgaatgtgtgcatatccgtgtatgtgagtatgggagcatgcccgcggtggtggttggcggtgggcaaataataaaattcaatttaattaaaattgaaattaaaactggaaataattacaaataaatcataattatatctgcggttagattgtgtgcaagctaattataaatcaatacccgcccgcgattgggacattcgcttcatcattaatggtcacaataatgcgggacaccggaatgctcggtagcatcggcctggcatacccctgtccccggaaggacaggcgatacaatttaaccaccaaacctgaccgttgttcgggctacgatcgccatcatcgctttgatgtgcacttgaactgcggcggcgttggcaagcattggaacggaacgaaacaaaaaaaatcaaccaagtgataaacacggcataaccagcacagaacataacctccagtaccaaccggatcagtactgagtttcgctctctgatccgtgtctttaattttctttgcttttttatcattttgcttttgttgcctttttgtttttcccagcgtggctcgattggaatgagccgtccggttcggtcggaaaatcatgtaacggcataattactgttaatatgtgcgcaaataaaaggtgcgattgcatagcggatcgagtgttgttgccgccaccggggccacactgtctaccgtccgctgcgatgaaaagtgcataatggtttcaaaattgaatatggcaacgcgtttggggaatgaatggaaatctcttcacacaagtagtttccggttgattgagccaatcgattaacactcgtttgtgtgtgcttttgattcgctcaagctgtgaaataatgcgccaactttggtagaatgttgtagttttttcttcggctactttatgtgagctgatctgattgctgaaacgcgctgctgaggatgccgttttctcaagggtgactgtgttgtgcggcagtgtgactgtgtggtagtaatccctacgtcacacacacacactcctactgtatgcagcggcgaaggttatgtttagcaaaacgcgtcccaactgacaaagggcttcagggttattcggtcaaattcagatcaacatgctgcaataatcgcgctgataagtcccgcacacggagcgccacttgcatgcatcgttgaatcttccggaacagcaaaacgacactggggcacgtatgtttgcagcaacacggctgacccgtggccgtgtgccaagcgtgcgcggcccagtacgtcagcgacacggccacagctggtacgatggatgctcagtacgctcagttgatatgcgctgagttgtgtcagttgggtggttgggttgaccaggcgctagtttacagtgtgctaggtggttggtcgggtgtgcctgtgaagcctaaatggaaccaaaaagaaggttcggagcaagatagaaataacaacaacgtgccataaacagctccggtgcaaatatgtctcctccagacgcgatacccaatcagcgcaccccagcccagcgggtagtatcactttatctagagcggaccggtgctactggtgctgccgatacgtgtcagaatgtcgtttcgcgcgctcgcgccctatgatgcttcgtgcgcccagtcggcatacactcctaattcgtatggataacgttacgactcgagcaacacgcactgcacgatctgtctgacaaacactctgccttgctagagcaaaccgctttattcttagaaggagagggaatttcaatagatcacgcgtcgtgctgcagcacggtgtccgattgtacaggttggaaattgtaacgctccaggaagtagcgtagcaaaagaccctcccgagtggatggccatgctaggttgatggacgccgtagtgcgagcgcttgcactgacattagcaggaagtaccgagttcaattgctctagtaatgcaatcagctaaaaacagtacaagaaggcgggtgttaaagacatttcaaacatgctgcagttgcggtgtgcggcctcgttccattgtatgcttaccatctgttcctcgtcgagcgtattggtgctggtggcgatcgattgcaccaaattggccagcgcgttcggaccgagcagactcacgacgtacgtgtagttctcggtgaggaattcgatcaatgcgtccaccccttggcggctgctgaggacggactgtagaatggatagccgttcctcggcgttgaagttcacctgcagctcgccaccgatggcagccagcaggtacgagctcagctgctccgtatcgttggcacatcccagtgcattgatcagcagttgccgttcacctcggttgtccgaacccagcagcttgccgaacagatactggaaggcgaccgttggcgcggttcgcaaaccgtaacagtacaccaccgccgaaacgtccgggtgcacaggttccgcgtcgaacacttcccgttccagggcgtcgcgggtcgccgtcatgcagctttctatttccattcggcaggcccagctggagattacctgtcggagatacttctccagcagtctctcgtccggtgctaccgttgtgatgtccagcgttacaaacacatcgccaatcaaggtgtcgacaaacagctcatagagaatgtaatcgggctgaccgcgcattcgaccgtggaagtagctgaggacccgattagccgcttcccatggaggatactcccgttcatggcgcacgtagcccagcagctcgagcgcaatctccagatcgagccgatttgagcgagccaaatggaaggaatcgtcgatcagctgcgcccgactgtgcattggaatggccgccgtgtcctcgagcagcgtccgaatcagcatgtaccagttcgagggatcatagttgacgcgatagaatcccgtctgattgacgttgaccaaaatccactcgttgttcggtgtgctggacggtacacgtaccgctttcgaagtcatccactgccactcgagcagagcgtcctgcgcatcgccctgctccatcatcgtgtacggtattacccaaaccgtgaaatcattattaactatcttgttaccgtagaatcggtcctgcgagaggatcatctctccacggtatgagcggcgaacttccagcacgggatagccggcttgattgacccagctatgaacaaaccgctccacatcggtcccctcgggcagcgatacgacaccgtcgaacgcttccgtcagtgcggccacgaagttatccgtgttgaccgtgccgaactcgttgccctgcacgtacgtgcgcaacatctgccgccaggcggcatccggcagcagcagccggaacatctgaagtaccgagccacccttggagtacgccacgttgtcgaacaggctgaggatggcattaaacgttgcgccgcggctgaaagtcatcgggcgcgtgctttccgcggcgtctgtgatgagaacacgctgcaccacctgaacgttgaacaggtcccgatactggcgctccggataagccatatcggcccccaggaactcgtacagcgtcgcgaagccctcgttaagccagagatagctccaccactcgttggtgataacgttgccgaaccactggtgcacgtactcgtgcgcgatgattgtggtgatggtcgtttgcgctcgatacgtcgtaacgcccggctcgaacaggaggacctcttcactgtacaagcaggaaatgggcgcaaatgttaccagagagtagcgttgacaaatgaaatgattcaccacacacacacacacacactcaccgatatttgcacagtccccagttttccatggcaccggcagaaaattgggtaagtgccacctgatccaccttgggcatgtaggagcgatagggtagaccgatgtgctcgtccagcgcgtccattacgcgaacgcctgcttctaatgcatacagcgtttggttgatcgcgttggggcgagcatagacgcgctgggcagccgcctcgttctcggtgtacaagaagtccgacaccaggaaagccaacagatagatcgacatgcgcggagtagtttcaaagtacgtaacaacgttgccgtctagatcactgaaattgcaatcgaaagttatttgtcacaaacacacctcgcaacgtcagagcactcgacaatcgccatacccggcttcggcaaagatcggcatgttcgatacggccttatagctgggatgatgtttaattcccaactccaccgtagccttcagggccggctcgtccagacaggggaaggcggcgcgcgcactaatcgcctggaactgcgtcgatgctacatatttgcgcgtaccgttcgcatcgagatacgagctgaggtaaaagccatcgtcatcgacgcgcagctcaccctcgaaatcgaggtgcaaaacgtacgaggccggtgcaagcgcacgacggatcgcgaacacggcaaactcgcgctcagcatcctcggtatagcgcagagtttccagaaacgtgaggttcgtgttgggattggatgcgtatagctcgttggaggtaatgcgcagtccgcgctgatgcacgtagatggttttggcctgctgccggatgtccagatgtatgtccacactgccactgtacgatcggtttccggtgtgcacctgcgtctccaggtacagcttgtagtgcgtcggcacgatgtagctcggcagtcggtaccgtagctcctgcgctgccacttcctgcaggctgaccggatcgagcgtgttcagtttccgctcgctatgctgcaccttcggatgcgccgcaatggctgcagagtgcagcccgattagaaaaacaccgcacagcaaatgtagccgcatgtctacaaacttgaaggttgattttgggactgaaatctccggtgcgaaatgtcgactccaatatccgtaatcgcaacagtttcggattgttttacgaccagatcgaccacaaacagttgctcgtgtacgtaccccccgataaccgaggtgtggggcaaatgccttaggaaaagcaatttctcacctgagcaattgaattatccatacctttgtatagcaagcggggctcgtttggattgagataagaagtcgattgagtgtaataactgccgaacaagagctaatcggccttaatcgcttatcgctcgctagtgagtaaattcgtaggggaataattgacgtttactcaatgacttgtgtgatttatatttgatgtttgataattcgcatctcatctaaaccaatgctgtctaaaaacgattgaatatcttattgacgtgggccgtttttctacatttttgaccgtttacttgcgcagtcatgattgaatttggctgattgtgaatcattaatcattccgtaaatatattggtgctatactactgtataaaggatagtagcttagtagctcagaagcttagtacaatatttgaacgttaaagaaaccaaaactgagtttgtgcatataacaaatcccaagtactagcgataaataacgctacgcaagtaatctatctgtccagttgtaaacaacatgtaataaaatggttcaaaatggcgcgacgaccggaaatggatcgcgttaaaacgtctgcctagagacatcttctttcgtatggtgtgtgccataacacctctctcgctcttttgtagttcgtaccacttagactcccgatgccgatgtaatactagagtaggaggaaataattaatatcacagttagggcacgaatgcttgcgtacttcacgaaaccttatgtaccgaaggtggagttgcgattgctcacgcgttgttgccccgttatatgcgaggtgggtcgtttcgggccaagatgtaacaaccccagcataaggtgggaacgagaaaccgtgcccgagaaaggaacgttccatctaagccagcgtggagggctctttgtgggcatgtgtacggcgatacggcaacccaaaagagaaagggcgaaattaatgtgtttggctcgttggccaaacagcagtcggtttgcacaaaaaccaaagcgcctgcgaaaattagtcacaccctcccgggccagcttttggggagagtgggagataatgttatgtgtctaaaatggttagacattttttacacgtgaagcaaagtttgcattcgctccgagcgggagcaggttgtgccatgtcggcttagggtgggtggaatgcgcgtgtttgtgtgtgtttgatgtgatgaaaaatgcaattgcgagcaaagtacgcgcacaaaccccgcaggccaatccctcttttttccagctcctttatacatttaattccagccaagcagagcccgccgttagccgtgctgtgtgagctttttacacgcttgagatagaaataatggcgtagtgcgctggttttcgttacagtccgctgcacaaacccggactaagggagggcggctgatggtggatcgctggtgccgcgtttacggtgtgttgcattaacgaggcccaggaataggcagaaatgtatttataattcagattagtaacaaaatggtggctctcaaagtgcgattgaagcgcgaagaagagtgcaacgaagagcgtgtccgtaataaatgtgcaaaaaaaaggaaccaaacatttttgcaataaatactgtttacagctgacggggtaaagtttacttccagcgttgcaattgcgcttgaatgctcgttcgacccggttgtgtgccgaactcgaagctttctagtttattttatgacaaaataacaaacaaaatggtgtctgtcacaccctgtaacctctctattaaactgatgatgtcacgcagcagccataaaacagacatcccactaagctctctatgatcgtaatttgtagtgcaaaaatgtagccatattaatgagtaccttgcaatcggacgacagtgaaggtctgccataaaagcgttacaaaataggcacagctctgggcagtctagtttctgcgcagcgatcaggcacactcataagtgcagctttgaagcgtaaactgcacttactaacgtcctgattcatcgatcgaatagcccggcacgcccccatccgtaggcttatccgggctgttttgctacgagcggttcaggtcgttaaaatcgatcgttaaaatattatgggatctgtcctcggctcttctcacgtgcattggagaaggtatggcgcggtgcagatgaagggatgccgaggaggaggtatggttcatatttgaccacagtgcgtatttgcgaaacccgaaaggtgcatcagctaaatggtggaatgtttctgcttttacgagtcgacagctgtggctccttcgacggggcagtcattaaactctcctcctaaaatgtcgtttgcactcaatagtggcagcactgcctggcccgatcgagccttcgccaaaagatcgaccgttaagggaggggggaggggtaaccgcgagcgatggataaggatatcggtggcatcgatttcgtttaatgttttgcctgctgcatcgcaggccgtcgttatgagccctccgattagtgcatcgtgataataagggcaaaacactccgttggtggcgctgcaactaactgtcggcaagaatgtggcattaatgccggcaacgacgggccgttttgtttaatttcttttcgtcgtcaccggccgactgcccgctttgccaataaaaccgtgcgtcgcgtgtgcgagcgtgtgttgcctggcttgtagcagtgcaccccagcccagccagagtgcgctgatcgctccaaacagtaggactattaaaaatcaattttccaccgatcctcacgcagtcgttttttatctctacctccgctgggggaatgatccgcgggcttgtctttacgcaggcgattaaaatgcaagtgaaaacaaaaaataaaaacacgaaataaaacacgattaaaatgtcagtgagtgatctttttttattattttcgttccacactgcatgcatgcgtacgctttttcagttttgtaagttcagaattggttcaatggccgatacggttggcgctcggtttgaagtaacgaccccgcagcataaaatgtgaatcatttgtgtgcgtgtctgtctctgtgtgtgatggcattctggtttttcaatgatgcgctcctattttcacaaccattacggaagggccagattcattagccgttaatcggaaatttgcgtggtgacgtggtaatttgtagtttatttatttgtgattgctttcggacgatgcccttttcccggtttgttttttactgcggatgtggtgcgtgtgcgaaacggcaggaaaggtcgactggttcccatcggaatggattcaaatgataatctgatttatttagcaatggcactgaggctgacacgagccccattttgtgtcacattgtagctgcagtggtaagttgccgtaaaactttaattcaattttcaactcaccggcaccggaagctcgtacagccttgacaaggaagaaaaaaaagctttgatacatttagtatttaaatggactgagcggaattttgtgaagtacaacgggcaatatttattatttattttagtacttttattgaatcgcttgcaaaaccagtcatcatcttcaggaagtaagaaacgacgttttcaagatgctttgactcatctgatgcacgtgatctcaacacaacttcctcacacataatgccaaggaaataagtttcactcaatcgaaacatgtttgtgtgtgtgtgtgtgtgtgcttgtcgaaaaacgctgctggaaaatatgcgcattttcagtttttactacctctccgaaaattcggtacggtttcggtgcggtgctcaccagcccgcccaaaagttacacgttgattcccctcggaggtcacgtcactgtctagcacggtggcggcgagagactggcgggctgaaagattgaacagcggttcgtcccaaaactaatccgtgaatcatcatccgtggccgagcgcgagcacggcgctgcccccgggagccaaggggcagtaaaacatgtttggttttacgagcttggaaaagtttttctcattttcctcgctcaaccactttgctgtggaacggattgcgcggcgctcgttagcgttttcgagatgcgagccgttgcctctgttcttcgtcttcgaaaccactgttgtttcgcctgtttgatttatgtgtgtgtgtgtgtgtgtgtgtgtgtgtgtgtgtgtagtttgtgatggaaactaataagttttgatgcttcctttccctgtttgtctgcatgctctttggtggcattttaagaaagcactgactgacaaaagccaagtttgtgtacgacttaggatggtcaaaccatagtttgggagggccttcatgtgtgtatgtgtgtgttttttccacactccgaccagtacgctagtgcaatgtagacatcctcccggtaagatgcatcttcccagcgagcagcggttgcgaaccaacgaaccttggcttgcatgtttttgatgagttttaaattttggctgatttggtaaatttttacgactttgtttatgaaacgatggaactgacaaaaggcacaccaggcaaaccagcaggaatcgagcgaaaagcaaatcgcgtaacgaaccgcacgtccaacataactgcgcaccccatctcgaacggtggacggtgcggggcacgtcttcgcagcattgcagtggattgatgtcttccagcagagttttggcgccgccgtccagcgcattgtgctggcgaaggtcggtgcaaatctgcaccggaacacggaagcacgaaaaacggaatcgaaagcgcagacaccgggaacgataaagatgtttgaatgcgtcataaatctacaaagacggtcagtgaaatgaattggaaactcgcatttgtcgtcgtcaacgtcatcgggagttgttcatttttttttttgggaggatagcaaacgcacatcaaatgcagtggcccatcacaagtgtgatctacaaggtggtggtgatgacggcggtggtcttgctccgtttaaacgacaatgtaaccaatacgtctagcagttgacgatgcatatgattagtgaagtggaaccgcgctttaaagacacctttgcttgcatgcgtgtgtatgtccgccagatcgcacaattcatcccaacgacatgtgaaggctttaaaaacaaattgaaatcgcttgaaacacatattcatagcgtgcccggccgagaatgggttttacttgctcgttaacgagaaagagggtgtttcttcagctgctcttcagcggggttagttttgcatttgaagcaaatcgttacaaaatgcaataaaatcgtctaatggtacggcgtaacgacgtgtagttgtacttggaccaattggccacagcgtgttcgccgcggaacacgggcaacacggggtggggttttagtttttattttacattttttaaatgcctcccttcgttgtgccaattgctgtgcgatctgtcaggtttcgaacacatttcttcgctctgtgcagcgaacgcgtgcaaatgagcgtaagcgtgagtgaatttcaattccaaaagaggtccagcctgtcataaaacctcactccactggttcccttttccgcgcggtcgctcgcccatccatcgctgatggcatcgaaaatccactcgttaaacgcgaaaccacgaaccgatcggcgcggggaaagggacaccggtgccagcggccgggcgcgcaaggatcgtaaattataatatgatttttattacattttagcgtagcataagccgaggccggctgagagacgttcgtaatttgttataatgttatatggctttccgttcccgagccgtgcaccgacacactgggcgccgacaagaaatggctcagggtgtactgtgtgtatgtgtgtgtatgcctttgctgctattgttatttttatatttccttccagtcgaaggaaacgggtgtctttggagaatggggaagctttgcacaattgtaccccagcggagactcactctaataacgttcattttcaacaaataaaagcattgcatcagaactatcgtcagagtgtgtgtgtgtgtgtgtgtgtgtttgtgtgctgctgcgataatttctgtatcgctttcgtcatcagttttatttcgttattttattttacaattgctcgtgaagtggcgtgcaaacgcaattgcgagccgctttggcgagcaaggaaccgcgcccaagatcggtttcggttcccttttctttgtgaatcatggttgtgaagatttgttgtgcaaaaacgccaagctagtaacgaattggtaaaataactgcgccactgcatgcacaaacacacacacacacgcacaggcagaggaaaaacgaagagtccggatacaaaattgcggttttgtagcttttatgatccaattagctgtagaacaagaaccgggacgatgcgaaaggggtgttgtaagacgcacacaggcacactggtctgggcatgctagtcgatggaaattgaatcagcggatatgcgttttgcgcacatgccttttttcatccttcccttttaccgttgaggcatgggaagtgtcataaactcgtgtatgcgatttgttgttccgtcaaggtttcgtttgactgagttgctgtaaatcaaaataaaataaagtggccaaagggccgggacgagcagaggaatgtttccaacgcatgtcttggtggtgtccaacaatcctcatttatgatgctgcattgtcaatggaatggtctcatgtggtccggacacgtccaatcacatttattgcttcattatgccgaacgaagttttatttcggaagtgtggaaagtatgtttttttaactcattcgaacatgttccttttcaatataattttgtatagcttcgacaaggaattcgctagcagttattcaacaaataattacgcatgcaataatttgtcgcatgcaaattccggtttcagcaaaagctggtttttaaaagctcgagtaaatgtgttcaacatcctgctatgtaaaattaactatgttttgtaagtgttccaatcagtcacagaacgccaagctgaggaagagtatagtgttatagaactttactagaagccagttggattttgttcatccccacactaataagacagacacaatttacatttgcgtagtttgtgcttttgcataatacatttaaatgtagaaatttaaataaatagaatcataacattatgcttctggggtaaagtacagctagcttccatccttccctacattaaaatcaattgaatgctgccatataattacgtgaaaagaagaagaaatagtttattgcggtgttttaccgctattattgcattacccgcagcaccgtcagtaggagtagtgctatgcttttacctaatcataaaactagttattatataccttctgcacacccaagtggcatgattcgttgtgttgccctttctccccatgctttgtgccgattcccaacagcgagtgtgagaacacccgtacaagaaaagccctattcttcccacccagagcgggaatagtatacgagagacccttgcacacttttccatcgcgatatgggtgtaatggtcggtgttggggtgaattttccagatcccctcaatattgctcgaggctttcgattggctcgggctgctgtaatagtgtgtaatgggtgtgtgggcactccagaagatggaaaccatttcgtataaaacaaaagaaaccaccccatgctcgagaccggtgcgatcgctcgaatcgctgaaactccaccgtcacgagcacgacgttgtctagttgggctggatctacaccaacctgtgctagtgcgcgcgactagatgtgcatgtaaaaaaataaacatataaatcaacaatgctcggcgtggcaagcatcaaagcaagtaacggatagaaagagcaaactcgagggagcaaacttcgacgccaacaaaccctcccgcgcgcgcccagcactagctatgcactcgaagcgcatagcgaaagatttacgcggggggatacggttggtgttggtgagcatgtttcgatgttgcgccccatgagcatgttttgggccgccagagcgagacgggaagagcgcgtgcgaaacataagacagaggcggagtcaaccctaccattggttgcgctcgtcggtcgttctgttgctcccgctctgatgggtggcgcgcgagcataggtctccgactcgctctagcgcgttgcagccgttccacacacctttttgcacgtgcggctttgccaccactggctgcggcacaaattccgaccgagcacgtggttcctctatctacatttctgcgccaaccggtggatgtggacgtctcctggcacatcggtcgaactgtgtgtgtgtgtgcgtagatacaacatctcgttatgttgtgcctccgaaagccgaacaccctcgaccgtcgtcatcgtcggtgtcgtcgcggttttatgctccggcgaaactgctgcgaacgtttcactctcactctgtcccagtgcatccggcacggtatcttttgcatcccttcggcggtaagtttgggcgttgcagcacgatgttacatcggagcactccgcaaaaagcaggcggaagaagcagctagcccgaaaatgtgtgtcggaaaatttcaccatcagttcgggagcggagaggaggccgcttttccgagggaatcaacaaacgatttcgctgcttatttgaagaagcagcaaccatctacgaacggtttcttcaaacgatgaagcacacaacgacataccattcggctctgggggaaaacatgttttagtgctgcttttcgccacgtatgtctaaaccgaaaaagaagaactttctctatcaacggaaagactatttttttcgcctgtttcccaaaccttaccatagaaagaaggactgcaatgcgcggatacgacaggaaaagaaccatttagcggcacatacttgggagagaagcacgttcgtaggaaacaaggatgtttatgttagcgcgaataattcagacacgctctgagcgctttcgggtgagattagcaatggagcattcgggcaaacgaaaagaacgtttgcgtttcgaatggggcgtttttgcttgtgcagcgatgacgagtacctcgtctaaaggcagtcagctatccggaaaacgttgctctcgattaatgcccgttggtagcatcgcacaatagcataaaagcacataagacaagtcaccggaaggctgcataacaccgaaaggttaggagaaaaaaaataacggacgataaacgggtacaatctgagttggtatctgagctgggaaaagggctgaagaaaataggagcagtagaagctttatgtaggatttgctcatcgaatgaacaacgtactaaagatcgttttttacacggcggatttatgttggaacaagtcgttaaatagcgagctttgttgggagtatcaaataaaagaaaacctcatcacttaccaagagcactaaaagagatttagtcaagtagtgttgttagtctttttattagcttgggatttactatttatacttatatatcttatctttacttaaaaaatggcaaaaaaagataaatagaaagatgtcaaatcatcaaacttgttacattgttttataaacttgtttgttactacttgtttgttataacattgctttatacacttgtttttactttaatgaaaacaaacatacaaacaagtatttatttttcaataatccgttatttttagttatatgactaaaactaatattgcaataaaatgactgcacttcttattggtgttgaaattccctgataacgcaaaaaatgtcattaaaaattatgtgttagctaactaactaacgccatgtttcaatgttgaaacaagcagatgccaaaagttttttatgattttttatagtacagtagaaacagacgatatttttccgatttattaaagttaaagtgcattcaaacggcatattggtttacgtttgaattgaatgtatctttatgtacagtttaatcagtcgactgattgtttcactcattggattacgtttgccttgaaagtaacatttcaacctgtatggcattgcgcacatctatttacttgtcatgtcgctcctatggcgctccatagttcccaccagccccaccgaaaagattgattaacatcttgacgggtcatatacttattaatgccgcccataaaattaatcctgcccgactatgaatcggacattgtacacagtgcagcgactctcctcccatgtacggtaacaaccatgttacctcacgaaggtcatgtccgcatacgcgccaaacatgaagcgtacctaagcaagtcgtgcaccaaacttaaataaaaataattgaatcaatcgagcacggcttgtgataaacgatccgattgattcgttagccggatgcagttgcagtagttgtcttgcggttgtggagttgcagtagggatgggggttgtggaggggtatgtacgtcagcgttgggtggctacgatcgcgccacgtgcgttcgcgaaaacgaccaaccagcaccggtcttatctgagattaaacgaacgaggtgacagctaaaaggagaaaccgggcgattatttaaattagttcccctacgaatgttgtacggcgcggcgggctgcatcggaggagggatcttatctcgggggtagcgttatttgcgttattgtaggcaaaaaaaggataagtatgctgctggtaagaaggtaagaagtatgcgcgctgcaataagcatccccgtgccctttcggcacccggcgtgtggagctcggtgcatcggaagctcggatttcagctgcaccgaaaccagatgcacacacgcgcaccgctccgggggcgttgaggcaatcgaaagcaatcaacatcaattagcaagtttatttgcaacaccgccggtttcgatggattcttccgcatcggcgactggtacaaattgctgctgctgcgcctttagcgggtggcagatcggttttgccgctaccggtaccgcatactatgaaagtatgatttatcgtctacaatcatttcccattacacaggcgcggatcgtaaaatcagctccggaaatatgtgtgtgggtttatgtgtgtgtgtgtttcggtggggatgaatcgaaaattcatcttttgctagcgggacgaagctgttggtgtggagtgcccgtgccaaatacgttgaaggtcgcgatgtacgcgattctctagccttgcttagtcattcagcgggaatgggttggttgttgcgctcgcattggaaaggtgcattctgcaccgaagcattccagtagcgcacgccgatcgtttgctcgattatggtttgtttagtctggatgaataaaatattgctcaattattcaatttatcgcgggcctgggcccggcagtggcaaacaggactgaaaccgccgttctgtgcaggtctgttccgcgatcgatactatcgtctgccagtgcatttgtgtgtttgttctggcccgcttgttgatatgttgtggttgcccgcttggcaaatgtgcaacgcatccgcgaatcgagatgttgcagcatggatggacacgaaacacgagccataactgtacaaacaaacgattggcccaagttggtttataattgcgaagcgtgcgttaacatggcgatcaagaataagttcataatcgatggattatgagcttgagcggaattgcaaggacacgaaattgataagcacaaacaatgaatgtgtattgtgaaagtgaatggaatttcaggtgattcatgtctgggaaatgtttgtaccacaaattgcatcataccattgagaagctacaattacgcagattaattttacgcacagaattgcagaaaggaactgtttttttttgcaaataaaaaaaaagattgaatattcaacagttggttggaactagcgaaaccaagggcccttcaacccgaagaataatgatacgtaatttttcacgatcgatgcaaaacatgcacaaaatattgcatttaattcttcacagctagcaccgatcgttttgtcatgatcagcgatcggtcgatgtgtgccgctgcttgcaagttactattctggtattcccattctctccggtactggagcagccagcttcgtgtcatcgacaaagcgcttcaagtgatgcccttttactacaacccacggcgaactgaaaatgccagaaatagatagaggaagatcgacaatgatctattgactagttcaggcgcgcgcgtctcgctaggatttgcttttcggaggatccacctcggcacaatctcggagacggcggtgatggcggctctaccggtggattgacactttgacagctctgatgcaatacccatttccagtcgacggatgacgcgaaatcgcacaaaatccaccctccagccggggcggaaggaggacgcttatttccaccgtgatcaaatgacaaacgggcgcgtgcgcttgtgtttagcaggcaggggagatgagcgcaaactgtgcaagaagaagcatcactgtgaagacggcaatgcaaagatagtgtgctcaacttctccgcgaagattgaagctaaattaagcacgagattagcatgactgaagtgacttttcaaagtgtcagaatggctgcactcgcaaactagctggatgcagcgcaattttgccccggtgtgtgcgcgcatgcaaacgagcaaccgcagagggcaaaggagaggatgggaaggagggagggagtgaaagagcaggcttaaggttgccctcgggcattgaagtcgatacagcggttctattccagtgccagtaacgatgacgaagacgatgttgcttctgctgctgttgctgctgttgttgttgatgatgatgatgataatagtgcaaatataaaataaatcttccgtaagctttgtgtagtggtgcgtggctactataagcccgtctggaagcaaggaagctagtcgggcagggtcatgcaaaagggagacaccttcggagctccggagctcccgccggcactctcggggggacgtccgttatgcgttgtgatttattatggaatatttattatagtgtcttgttttgaaaaaataacttcaacggttcgaatttcctacacctcgagatcggggctggagtggcaacgtggtacggaacggtacagcggtttgagccgttcggtcttgggactcacggatcgcagaatgttattgtgcgcgcactgatgggaaagtcatttttcaccgagtggtcagggcgcgtagtccagttcgtttctggctgctgttgctgatgctacgatcctcaggaatgattggaaacgcctggagatggtgggaaaaaatcaaacacaaaaacgatcctaatgaacatcgtgtgttctcattcgctgccacgattgacaccttcgataagacgcacataatgagctaaaggagaggggacagggtcttgtctttgccacgagcgataagattgcaatcactcgtgagcgtgtgctgctgggctgaagaagaaacgctttccacagcagtaggtgggaagtgggattgtggaacgtggcattgaaaagaacctattttctaaagcccgagagcccgttctcgaactggaaaaccagatgcagaagttttttattgtcccccgccaggaaaacaaatgtatttaatgctttctttgccttttccgccccgtttcagacgacgagctagtgaagcgagcccaatggctgttggagaaactcggctacccgtgggagatgatgcccctgatgtacgtcatactaaagagcgccgatggcgatgtacaaaaagcacaccagcggatcgacgaaggtaagctggcgatgatggtgtcgttcgacatcactttcatcaccgtgtcagacatctactgtgcctagcaccgggtccagtggtcacagggtgtagcaaaaacgtgttcttttttgcgagagactctacctcatgatgcagctgttaaggaaaggtttcagatgaaggcaatttttcctaggataagatgatcttaagttacctgcgtattagtgtttaacattgtcgtctcaactcccaagaatgttttaatcgtctagggctagtttatttatactgttctcattgaaatgtcgttcaatccaacatgttaagttagctagctcagacacgagaagttaggagtatctgcatcttgaaggtagcggcatatggtgttatgccacgttcactgacttcaaaattcgatacaaaaaaaaaaccaaaacatcaaaaaccaaattgtgaattccgtcagccagcagcagtgaccttcaaagccttacctttccattcatttatgtttaacacaggtcaagcggtggtcaacgaatactcacgattgcataatctgaacatgtttgatggcgtggagttgcgcaataccacccgtcagagtggatgataaactttccgcaccactgtaactgtccgtatctttgtatgtgggtgtgtgtatgtgtgtttggtgaaacgaattcaatagttctgtgctattttaaatcaagccgcgtgcgcaactgatgccgataagttcaaactagtgtttaaggagtggagcgagagagccgcaccacggtacagaagggcagcagaatgggtcggcagcctagctgcactggtgcggtgcgtccggcgtctcggggggagggcgaggaaattctagtgttaaatcggagcagcaaaaacaaaacagtggtcgtcccgttcaagaaacggcctgtacacacacacagaaaacactgcagcatgtttgtacatagtagatcctagagcaggtggtcgttgctcctcgaacgctctggacgcacggcttcgcgcgtatttgcgtagcgttccgccgatcgtgggtattcgtactgccacaagcccgctttctcccatgcaatctctgcaaccaaaccaacaaacaacaacaaaaaaccaatcgacaaaatgaatcacaccccttttgtatcatctgtatattcttgttctttgcgttcttttctatgtggcccacgccccggcgggtacgtaattgcgtcgaaaaccccgaaaaccccggcacatacagtgtacatacggtttgaggacaactttgacctgcagcccttctggggttgccacgtgtagctatacttgtgagatcgggcgccgacggtgtaaagcgcgaatggccgccacacagtgtgtccactccaacactacccctctggaactaccccgtccagggatgcaccggctcggctcatgcccctgcaaaacagtccgggctccactgtagtagctccggcgttgctctgagagaaggatgcccttcgaagtgtcgaaagcgtgcattgggcgttcaagtgtgtgtgtgtgtgttaggtttagcgagaaacagcagcagttgcgtgtgctgaaaagcgaaggagtaatagagtgcataatgaaaatgaaaatgaaaatgaagcaaaagtagaaggcggaggagagcaacctgtgttccactagtagcgaatagtttagtctagtttcgtcaccaatcaaccttccaaccatcgttcaaccaatacctgagtcaacatcgtcatcgttatcgtgccacaactttattaaaaatgaaccttgtccgcgccaccgtagggtgatctaaggcgacctttcttacgggcgcgacccacatgccatcgtcaccttctccaatcaaaaccaacagcctgtaccgatggtgtgcaattgtgcgtgcgtgtgtgttattagcaaaaaaagagaaagagtcgacgagagagagatagatcgagatcgagagtacaaaagagcagtagaaatgttcgttgtttgtttttcgtaacacagttgtttagccaaaatgggaatttccaataatcccgggggcggggaaatgcgggaatactgcgtacacacatacatcaatcaaaaagaaaaatccttgcgctacatcactaccgtttgcgcggtgctgatctagagcagaccactttccactccactctacaatcaatcaatctgtgcagaaggtatggtaagacggcctttgagcgagtcacggtcgccaccataacgccgtccgacgagggctgaatgcgaactttgctaatcgattttccgctttctttttatcccacctccttttctctccctctctctcttttgcactgccccttgtaacccccaaaaaggtaaacgacacattaagacctacgaagcgttggtgaagtcatcgctcgatccgaacagcgaccggctgacggaggacgacgacgaggacgagaacatctcggtgacccgcaccaactccactattcggtcgaggtccagctcgctgtcgcggtcccggtcctgctcgcgccaggccgaaactccccgggccgacgatcgggccctgaaccttgacaccaaattcaaaccatctgccagcagcagcagcaccggctgcgatcgggacgacggtgactgcagcgcgttcgacgacagtgcctcggtggtgcgggggcacgggcggacggcccacagcaccggtagcaggggccgcagccactcgaaacggtaccacaccctcccggccgagcacatcgggagccacatggcggccgcccagagtcgatcgcccgccccggacgacgagccggtggtgtcggtgtccgtgtacgagagcctggtcgaagcggccagcaaaaagacgcgcaccttcagcccgccccggggggaggcggaagatttgcatgccgcacggaaagcatcgccccacgacgagcgggacgagccgaccccggcccagccctacgaagcgtacctggagtcggtgcggcggagtaaaaagtgcttcgcgctcaaggacagcgaggcgccgggcgaggagccgacgggctacgagaaggagaaggagccgcgcattccgtactcgctgccgaagagcaccttcgagcggctcgacctgctgaagaaaccgaacgggctgacgtttccgatgtacaagtacagcgggatcgagccgaacaactttgccctgccgctgctgctgcccgggctggaggcggtcaaccggacgctctactcgacgcccttcccggcccagctcctgccgtccagtctgtatccgtccgttagcagcgagtccacgacagtgcccatgttccacacgcactttctcgggtatcagccgccgctgcagctgccccacgtcgagcacttctatcggaaggagcagcagcagcagcagcagcagcagcagggattggccgaaccaaaggaaccgacgtcgtcgtcttcgccgggcagcaaccggcttacgccaccgaagggtgcatttttctacgcgagtgcggtggaaaattcgctcaccgcccagcaggcttccattgctaccatccattagatccacactgcgtccactcgctgtttgctgcagcgtaccgcggacagtgcagtgtaccgctgtacaaaaaggtaagtgtgggtagtaagcggtagggtgggatgggtagattagacagtaggcaagtggggatgcaaatttacagcccttttggtcactttaacagacacaacagacaagggacgctagcacgaatcatcgcaacaaaatggaatgaagcaaatggcctttggacattctttgatcttcacactgtttccgcgggctggggacgttattagaggaaaaacgccaatatgttgtcgtcaacattggttccgctcccagcctgggggctgctttacttctgccagtatcgatcatcgcctggtatcgctcggcattaaataaatcattcatggccaaatcaacgtttagttattgatatgggcaggaggaagcaaacaaacgaaaaaaaaacgggcacactccatcgaactggatactggaaactctgcaccctacgctcaccctcattgcaccctaccagagccgatatgctgcaaaattctaaataaaaataatccatgcgggtcgcgaagcaaataatttatttcctatttatatttatttttaatcacacacaaatatgggtgcatgcacgtgtgtgtgtgtgtgtgtgtgtgtgtgtgtgaccgagtatggacggacgatggacactgtggtgcaaatagcggtgagcggtcgtggccgaaggttggctaatgcaacgcgttgtgtcgcccgtttttccgagcgtgcctgatttccaatgcctatttttcactccactgccgctttggtcgccattgccttcggggggacctttttaaggcaaatgttgatttgcaccgacacacaccgaattgcacactgcacccagtcagtcaggcaggtggtgttgtttgaaaatggcgctctggagcaaccaacaaacgaacacaaaacaaaaaaaaaacaaatcaatagaaagaatcgagctgtttcgattattcaaaatttatacacaaaatatgcaacgtattccccggtggggtaccctcattgtccgacctactcccccccggtgcacctcaaacccaccggcagcaatcaatgtaataatggtaaagggtggcgtgccaaatactcccggaccattccgcgctcgacgtagggacatacagagagcgggagctgcagtgacacgagtgaaacaacctggagacccctgcattcgtcaggcggaaataaacaaatcaaaacaaacctcccgtctgatctcgcgaccctgccacccaccggcagccggcaaccagtcgtccaatttcggcactttggcggtgtgcaactttagcagtctatgcacatgcattgtaaatatgcatattgcacgagataaagagagacgggccgagagaaagggtctctgtgagcggggtagccagaagtatcgaacgacaaactatgcgcgtattacgagatgcgatcggtttgacactcggcattcgcactttggtggctatttttattcgcctgcttaactccgtcgctgtttgtgcgtggctgcgtgtatgtggccgggcgagcgtttgtttaatctggcacggtgcagtatgcagttcggatgccagcgctcgccgccccctgcaccactgaccacccgttccatgcccaacgacagcaacgtcccggcagagtgatcagcagaagaaaggcgtttcgtgccaattctgtcgtatacatcgtgcacggacgcggattgttgacgaaaggttttgtagcaaaccgggcggcgaacaagttatgaataaatttactccattcgttatccactgatgtatcattaatggcagccggtcagctatggggcgctatgggcagtacagtcggtcccgggtgtgccgatcggtaaataaagtgatttttgcattccgcttccgtggtagctaattttgtgtggcacactttggagcgaattgtttgattagggctcgtttgttcgcttgactgtaagctatcatccgatgaaagcgggcttaaatgctagatttactaggccgatcattttgacaggtagctctaggagcttttcattatgcctaattatattgtaaatatttagttgtgcatttaatgcaaacttccaacaaatgaaaaagtcattctgctcttttaagtattttaatcagtattttcaaagctttaagcacaaacgcttagaacgtttgatgtttttagtattttatctacttatttgtttattgagtgcccctgacattcgtcgctcacaaacaataaatatttttggacctggatctagtaaatgtacgacatagctcgaattgaaaatcaacgtcaatatctctctaattttatggtctaattgcatagagaagataaaaaactatctattatttaccgattagaaattaattctagtatcctcctgctagtgctcgaatcgaattcatttgcattccttctgcttgctagccgcaggtacagcaatatcggaaactctttctttaatataggtttaaagagcctctaatgtgcatctttgcgctgatcgtaacgtttcaccgaatcatcaacgagtgttgttttgccttctgcaatgaaaccatcctacactctcacgtgtttgaaagaggtccacggcacaccgggaatgcattatgcgctgacggcggtggtgttttgttcgaagttcgtgatgcaaccgccggggaagttgcacacagggatttaacgactcctcgtaaaacggtattatatatcgaggccgcagcgaaaggtaacgccgcagccgcagcaaacggctacacaaaagtaaacccctctctgccgcactcgttgcgcagtgccggaccgcatggcgcacatcttcgaccagttcgcgaggtcgctcaatacattaggaactaatatatattccaggcaataataattttctattttactgcccttcgtggggagatgctttgcgagtggtgctctgtgccaggagaggcagagaaggcatacccaccaaccacctccagggtttcaaacacgttccctgcgcttatcgtgaatcttttgcatcttttgatgatcgatactcctcgggcccgggacaagaccaacgccaaggtgcaccgtgtggaccaacatcgtagacgacaatccgtgcgttgcgttttggcaaggaggagctgtacgaggtgagatagagtgtgtgggagaaagatagggatagcacaaaagagtgtgtgagagagagagagagagcgcacctagaataacagctcgcctgactgacttgactgactggcaggccatagaatggtggcgagaaaaagcgtcttacaagacgcgctaaatgcaactttacaacggtcgtaaactaggtcgtaaatatctttgccagcataccttctgcaaaagagcagatcccgcaaacacacactgcgtacggcgcaacggctgccactcgtgatgcacttgtagtagaccggggcccgatccgaaccgtcccggacgcgttttgctgaccgaaacagacacgcacacagggtgcattttgctaatttttatgctaaatttttccaccaccgacatgggatagtttccagctgagagtgcaagtgcacttggggtgcaagttgtcgcatggagcgcgataacggacgcagtccactgctcatcttagccttatacctgctcctggaagatccgatatgtctccaatcagtatcgtcggcagtattttacgataatccgcagcgaacgggaaccggccgccttggtagcggtttgtcaaacggatctgcactccgcactaccgtcatgacgcgattagaggtagagcagcatgccgtactacgctaccacttgcaacggcaaacgtcgcggagcaacattgtggccgcagcgccgaagcaataaaagttggaggacatctgtgagcagataatttacaagctactttgtataatgaaaaacgcattaaaaaactacgcctggcaaaagttcctagttgttcttaggggggaggaagttggaggggggcaatcatttgcgaaccagactgcgaaactgttacaagacaaacccggagcatttccgggcgatcaactcatgattattgttagactcgcggtgacgagctgtgaagcgtcctgccttttcggacgttgtgcgaaatgtttcgcactgcagcacggcgggtgttcgatgccgtggtgtagttgcggtttttctacagctctcacatacacataaccggcatgaaacacggaatgcgagcgatgcgagctgggagttggcgcatcaaactccactaatgttgcacactgtgtggggtgggatcaacttcttcgccggcgtttgttaccgcggtggtgccgatgaaaagacgccatagatggattttagccaaagacacaccgttccatcgtggccgaacaacggttgcaacggtgcgctgggcagaaggtaatggaaccggttccggtactgatcggccattacgggctagtgaattttactagttttcagagataattttatgggtttccatttgtgggaattgctttttttattgcctcaactggctgtgaggtctctcttctgggccggtgtgttgtttcagcagtttcgttcctttgttcgagcggttttgtgcattgtgcttgatgatatgacaaacccagaaaacaaaacaaaaaaacgataactacatgcgtctggtttatctggctgtaaatttagtttgcagtccttcaacacacagacttacacaaacctcataccctaatcattgtgatggatatcgttcagtatcacgatgttattgaggtgtgttcacatattcctaatgaattacattttttgttttatccattttaaatgatgaataaatattctacaaacatgtataaactcatattaataaacctattgtccaaattaatattaagtggcgtgaaacgatacagcttatgcactacgcaaatattacgagaatatgatctaatttgcagtgaaaatttgttttccttggttccaatatttccacaaccttatatatcatgtgaattattttaaaataagttatcatcttagaaaaaaatcatcatcagatcaaacatcactagatctcaaagttacatcaagccgttcgctctgaattgtagttttatttcgagtgtttcaaataatttacttttttctcatcatacttatacactttttctcgatttctttccgcttcctcaaaatagatcgattggaaattcacgtcaatcatctgcaagcccgaaagatgctacctagtcgtccccagctgttgctactggagctttgcaagagatccagctttcgttccttatcgatgcacaaaaggcgcacccggaaacaaaacaaaaatccaacccactcgtcaacggcccacatggcgggttgcactggagaaactcccaccctcgtaagtgctatctaagcgttaaattaccttcgccctttgcggtagaacaaaatagaagcaaatgaaacaaaaaaatcattgccggaggcgcaagtgaacagcggaaagggaaagaaacccctgtcgaacagaaaacatgattattgatatttttcgatcgtgcaacgaaggtctacactgtgatacaaaatgttgtgtacaggataaatattagatttttttgtttggaaaacaaaaacacagctaaacggtaggaacaaaacaaggcaaaccgaacaaaacgaaacagtacgcacacggctcgttgtatgtaaatcaatctatgtgagcgtgtgtgtgtgtgtgatcgtatgtgattatgtgtgtggcgaacggtttcccattttctgtgagtaacgccccgttacgatcattgctgttggaaaaaaagctaaaaccaaaccttcatcgaaacgaatggcgcgcgttctttacttggcgcccaatttcccaccaaaattcaaacctgtttttaatagtgtaaaacgtaatgaaaatagtaaacgggcgtgtgttgtgtgtagcatggttcgatcacttggaaccaaaatctcaaaaaaaagcaaacagaaactcattggcagaaaggcagacacaccggaattgcgaagttgggaaagcagatcactttcttgttatgtctgcgtttatttctcgtgtgcgaatggaaggcaggaaattcagaggttcatctcccatggaagatgacggaaagagattaagaaattcgaaggcaaatctgttacaacggcgagcgattgtgttatggctagtaaagaattgaattgtgatacgtgcgcagtactgcatatttgttcaatttgtagcttgtaggtagatcgccgtcctcgtgttccgtgatccgggggcgggatgatagactccgccacttggagcgatatcccatgttgctgtactctcgtttcggtgcctttttttcttgctctttcgttttacaaaaaaagtaattatattgcttttgttttatgtgcgcacccgcacacacagctgcacacgatcgtacaagttaacgaatggtttagtttgcgctaagtttgattggttctagttcgctaagttagtctgtagagagattcgtttatcgttatgttcagcagcagtgtcaggaacgagattggaagataattacaggggcagggcagatgagcaaagggggtacggttaggggctggaagtcaaaatgctttagccatcctgcagtcgaatttaaacattaaaaaacaggtccgccttgacgaaacaaatacccccgaggagttcctgcgcccggcccctcgaatgtgcacgaaatggaataggtgttgtacaggcagaagacagttgtagaagcaagggtgtaatgttccaattgaaaagcgaagagaaaacctaatgtaactacaaggcagatatacagctgagagctatattttacgcagcgaaatacaatgtaatcccattttctccactcatcaaaccttcattagtccttcacatttcacacaagcaagttgtactataatgtagaaaaaagtagaacaagcaaaccatttgatgcatcatcgtcatccagcttgaaaacaaatagatcaaattacatagaactggcaatgtctattgatacgctgttcgagagacttttttttaacacaaccgtaacatcagtggtgccgcgtgaatgtatgtttatttctgagtataaagaaaaaacaacaatgtgcatatatactggtgtgcagtcagctctttctgagagaataaaaaccttaacatttcgctttgcacaaaccatgtcttgtaaaatattactccaacaagaaggacagtcaaagaaagaaacaagaaacaaaacgttaaacttaaatcaaaagctagaaatgcacatgtaccatacattattgcccagaaattatctcaacaaaggggagaacaaaacacagttacagccaacagaaaacagttacagcaaaggtgtacatagcatagagtcacaacacaatatgtacattttacccggttcaatatcaaaataaaatgaaaaaaaaaacgtcccgtccgctgatgacggagtaatgagacgaggcgtgaaaatgaaaatgcaacatcaacagttaagaatcaaaataacaaaaaacacccttatccggctccagtacacaatctattgatgacgaaacgtgtgctgcgaataatgttttaacaaaagatgaagtaagtagaacgtgtttgatggaagcgatgggcagcaaaggtaacgaaaacacacatgctaaacgtcatgtgtagcatgtgtataatagcaagaagaaatttcagagcaagacccaaggaaaagtatctttgattcgtcaaacgccgcaaaacgctgttttactgctgtaagtttgagggaaacaacctccggtaaaagagaaataaagtggaacaaagcaaacaaacaaacaaacaaacaaacataaataaattattaatattattactgaactccgtcgtgcgtgctgtatttcgagtcgctttgctcgccaatgtatgcgtccgaaacgatgtgtttatttagttatttttaccaccaacaaccagatggtggtgaagttcaagaaaaaagtagctgaacgcaacgctgcgtcaatttctctgtctccccaccgcctttctctctctctctctctctctctctctctctctctctctctctctcgctctctctcgctctctctctcactctctctctcactctctctctctctctctctctctttgatttcatcggatcagtctgaactttgccatccaaacaacatttaattacggtcgtcggtattgaggcatagttttatcaatcctggcagcgggactcgaatagagagatgcacttttcccttttccatcggagtaaggacgttgtgaggatggcaaaattaggttgactagtttagcaaagcggaggagaagagttttcaatggtttcaccgttcttagacgcgatttcttcttcccagctggatgagccacagtttgagccggtcgcattgtactgtgcaaggatatgaaccggaatggtggcggagatgagtcgtgctgatgcggttccatccagtctccagacccggtaatcggtccttggccctctacctttctgaaacggtcctctgcaaggtagaaaataggtggttttctaccccgttttgtcttctctcactcttgcgtcgttgtgtgcaaagtactaccagaagtacaggcaatcatgatgctgagatcgtgatgctgcatatccgtggcgcgagaacgaatcttcactttgcactgtacgggggaaattgccataaaatgcgacaagcggtacggtggaaaacaaaactgtgcattgtacgcttcaccgaaagatgccagcgaacgcgggcttgatgctttcgtacttcgggaagttttcttttttttatttctctctcaattggagtctgtccttcgtgccgtggaaaccccgtaatcatgcagcacggtaccgagagcgtggctcaggcacgaaccgtcgcaaacgtgagcatgtgtgtgggtgctgtgaaatgggaagcatcgatacgataagaaactccagcaatcgattgtgccagggcgcaaagccggagcaaacataaacatgcagctcatcaaggatgggttaaaggagtcggcaactaaccggctacagaacgaaacagtgaagcgcgaagaagcaattgctaaccgtgcggtcccttgcctgaccgaacaatagtgaagctcattttccaagcgacgttggttggctgtgtgggctatggggtaaattttaaaacttcttttggggaagtttttggaaggaaaatttcattacgtttcaccctattcctttgcaagagcgggtcgtgataagatctctcgatggggacgtgctgcgagacaggttgatagtggcgagaaaacgtttgacgagcgatatcattgaaaactatctgcaaaatgcttcaccagcggtgtgcacttagatgctagagtttagttttcgttgctaggtgtgcaagtgtgcaaaaaatattcttacaatcgcttgttacttaaattttattacagatagcgaacaaagaggatgttatgtttcagctacataaatttcattcaataagtacatttcaatggtaaaacatctcccttgtgttaaaatctgtacaattgttgagaaatttcaatgaagtttataggttactaattaccgtttattattcataaaataacaacttagcccctggacaattcacggatactaggatgtccaagggtatgtgtgtaactttatcatagaataatttgttatcctaattacttcgttttaacagtgtatcgctcagttctacgtcaactatccgtggttcagtagctgaattcccgcgttggaatcgcgttggttctaggttagtatctcatatgcagattggttaacatgatagtcaataatgtttaaatccatgactgaacattgaagaatatgatacattttatgctattgctattttttttaattcatcacataccacacggtacattattgatttcagaaaggcatattttgattattatataattaaaaattacagctatttttcaagtaaacaccaagctcatgcattaaaccacaataaaattgattttttaattacactcaacacgctaacattttttcaaaaaataacattacatccattacatgccgttgatgaatacataaattacgccttgtttttgatgcacgataatttttattttgcgcaccttttgcccccggtcctatacaacattaccatgattcgtacgtgttcccgctcggcaaatctcgctaatcaaccgttcaacaatccatacatacccgacgttgatcgcacacgatgtaacgcggaccggctggagcgattttggcttgcccgactcgacacaaccgatcgacatcaattgcagggattaccggcacgccatcatcaaccgacatcgcctcggcaaacgcagctccaatcagcaggggctaatcactcgaagcagggatgcccggggagcagagagaccagaaacgctacattatccacgcggctgctattaagtttcgcccacaaccagcgcgcacacaataatcgtcattgatcggcaccggcaaaattaaacattggcaaacacaacggcaactacaaaaactccgatcaaacggtcacggtctgaattgagctcaagggggatggagagcgagtgagaaagaggtgagatatcatattccaatcgattttattcaaattcttaaataacatttatcttcccgatagctgattcattgccgtcgctcacgcctgcttgtctgcttccgctccgttcgcgttctatttgctactgcattatttctgctgatgcacccaatcatcctatctcccaccctctctatctgtactgagcaccgggcagggcgaaaaagggggagcggcagcaaaatgcattccccggagaggaacaagaagaagaaggcggtgcaacaaaaaagcaaacccggatcatcccggctcggtggaaaatagattacattatttgtgtttcattttgtagtatatacgtgtgtgtgtgggtgtgagtgtttgtagtttgccttaaattgttttataattactcttgtgcgacaaaacgcccctgactagagtgggttgggagcgaacaccacaatcgtgaactggacgggagaacataatccgatgtcctcgggtgatttgatgtacgccagggaaagcggatcatcaaatggtgtatactggcaaatatgcaaaaacttcggaaaaggggaactggaacattgaaacaagctattatgcaccttgcactttgtcccaccaactgtccagcaattcgaaataaaatgacagaagcgaccgtacattacactcccatttttttgtcttattctacatttcaatacttttcgccgggtgtttgacgggaatggaaaaggtgtgaagcgcgttcaatcttcatcatcctttgcccacatctcgacctgcggacctggcgggccatgtccatcaacgggcaagctgcagcgcccatcaccgccgctttttgttacccgtcgactcatcttccggtgcgggccagtgcagtcttttccttttttacgctcgctctctctcttaaacgcttccaatatttgtgtttaattattcgaacggaatcctctctgcgacagcacatccgtacggggtgccagtagtgtgtgcgagtccgtgtttgtgtgtagccgtaattatgttgtgattgtcattgtcactcgatgcgcgataaacaatctacctacaatttatgcacccactgggcggcctcgcctcgtgatccagtccggtttgcaagtcgccgcaactccaattcaatgtcatccgttctcacagcgaacgaacagaacggaggggacacgaacgccaacaacagcaacagcggcaaaaaatgcacccaaagtcctggatgctggggatgacaagagccgccgatccggcctcccaccacacaccaaacgcacaatcgcagttggaattgcacggtttaaatatatacatgttgttgctgtttttttgttttgtttttggcgtgcaactgtgctgctcctgctcctatcgtgcgctatcgtggctggatcccgcggggctactcggtgcacggtctaacgcatccggacgagcgtttggtttggttccaatgttgcagttgcagttggagttcgggtcggggacaaaaaatcacttacttccactcgagcgccaccgcgccggaacgaacgcggaaacccgttccacggtccatcatactctctttcctccctccccaaccgtcgctcagttcaacatatggccgtggggatcgggattgggagctgtcaggtccaggtgccgcgggaagggatcctgcagggaagtatcaagcgccggaactggaagcacccgatgacagatggtgctcgaaagtgaactgtaaaactggacgcccatcaccaacaacatcacaccggcatgcagtgcgacaaaaaaaacacacccacactgagagagaaacaaaaatcacatccacgcccgtcgtcatcaggggcgaaaaaacaacaaaccacacaaccggctgagccaacagaaactaacacagcgcgcactgggctggccacaaaatgtagtactaactaaatccaatccaaataattatatttcaattgtttatgaacggcattatgcgaccggaccggaaagtcgctggctcgactcgtccgtccagtcccagcaacaatatcaacaataacacatgctcccggcctggaacggtgggtatgcgtcggcggcgtatgctgaccaacataatcaacgtatcctttgtggtgggattccgggattccggcaggatccgc

SEQ ID NO: 2 - DNA, 129 bp, Anopheles gambiae
cctttccattcatttatgtttaacacaggtcaagcggtggtcaacgaatactcacgattgcataatctgaacatgtttgatggcgtggagttgcgcaataccacccgtcagagtggatgataaactttc

SEQ ID NO: 3 - DNA, 54 bp, Anopheles gambiae
cctttccattcatttatgtttaacacaggtcaagcggtggtcaacgaatactca

SEQ ID NO: 4 - DNA, 23 bp, Anopheles gambiae
gtttaacacaggtcaagcggtgg

SEQ ID NO: 5 - DNA, 20 bp, Artificial Sequence
  Nucleotide sequence encoding a nucleotide sequence that is capable of hybridising to the intron-exon boundary of the doublesex (dsx) gene
gtttaacacaggtcaagcgg

SEQ ID NO: 6 - DNA, 97 bp, Artificial Sequence
  Nucleotide sequence encoding a nucleotide sequence that is capable of hybridising to the intron-exon boundary of the doublesex (dsx) gene
gtttaacacaggtcaagcgggttttagagctagaaatagcaagttaaaataaggctagtccgttatcaacttgaaaaagtggcaccgagtcggtgct

SEQ ID NO: 7 - DNA, 1074 bp, Artificial Sequence
  zpg promoter
cagcgctggcggtggggacagctccggctgtggctgttcttgcgagtcctcttcctgcggcacatccctctcgtcgaccagttcagtttgctgagcgtaagcctgctgctgttcgtcctgcatcatcgggaccatttgtatgggccatccgccaccaccaccatcaccaccgccgtccatttctaggggcatacccatcagcatctccgcgggcgccattggcggtggtgccaaggtgccattcgtttgttgctgaaagcaaaagaaagcaaattagtgttgtttctgctgcacacgataattttcgtttcttgccgctagacacaaacaacactgcatctggagggagaaatttgacgcctagctgtataacttacctcaaagttattgtccatcgtggtataatggacctaccgagcccggttacactacacaaagcaagattatgcgacaaaatcacagcgaaaactagtaattttcatctatcgaaagcggccgagcagagagttgtttggtattgcaacttgacattctgctgcgggataaaccgcgacgggctaccatggcgcacctgtcagatggctgtcaaatttggcccggtttgcgatatggagtgggtgaaattatatcccactcgctgatcgtgaaaatagacacctgaaaacaataattgttgtgttaattttacattttgaagaacagcacaagttttgctgacaatatttaattacgtttcgttatcaacggcacggaaagattatctcgctgattatccctctcgctctctctgtctatcatgtcctggtcgttctcgcgtcaccccggataatcgagagacgccatttttaatttgaactactacaccgacaagcatgccgtgagctctttcaagttcttctgtccgaccaaagaaacagagaataccgcccggacagtgcccggagtgatcgatccatagaaaatcgcccatcatgtgccactgaggcgaaccggcgtagcttgttccgaatttccaagtgcttccccgtaacatccgcatataacaaacagcccaacaacaaatacagcatcgag

SEQ ID NO: 8 - DNA, 2092 bp, Artificial Sequence
  nos promoter
gtgaacttccatggaattacgtgctttttcggaatggagttgggctggtgaaaaacacctatcagcaccgcacttttcccccggcatttcaggttatacgcagagacagagactaaatattcacccattcatcacgcactaacttcgcaatagattgatattccaaaactttcttcacctttgccgagttggattctggattctgagactgtaaaaagtcgtacgagctatcatagggtgtaaaacggaaaacaaacaaacgtttaatggactgctccaactgtaatcgcttcacgcaaacaaacacacacgcgctgggagcgttcctggcgtcacctttgcacgatgaaaactgtagcaaaactcgcacgaccgaaggctctccgtccctgctggtgtgtgtttttttcttttctgcagcaaaattagaaaacatcatcatttgacgaaaacgtcaactgcgcgagcagagtgaccagaaataccgatgtatctgtatagtagaacgtcggttatccgggggcggattaaccgtgcgcacaaccagttttttgtgcagctttgtagtgtctagtggtattttcgaaattcatttttgttcattaacagttgttaaacctatagttattgattaaaataatattctactaacgattaaccgatggattcaaagtgaataaattatgaaactagtgatttttttaaatttttatatgaatttgacatttcttggaccattatcatcttggtctcgagctgcccgaataatcgacgttctactgtattcctaccgattttttatatgcctaccgacacacaggtgggccccctaaaactaccgatttttaatttatcctaccgaaaatcacagattgtttcataatacagaccaaaaagtcatgtaaccatttcccaaatcacttaatgtattaaactccatatggaaatcgctagcaaccagaaccagaagttcaacagagacaaccaatttccgtgtatgtacttcatgagatgagattggacgcgctggtaaaattttatatgggatttgacagataatgtaaggcgtgcgatttttttcatacgatggaatcaattcaagagtcaattgtgcaggatttatagaaacaatctcttatttatgttttgttatcgttacagttacagccctgtcctaagcggccgcgtgaaggcccaaaaaaaagggagtccccaacgctcagtagcaaatgtgcttctctatcattcgttgggttagaaaagcctcatgtgacttctatgaacaaaatctaaactatctcctttaaatagagaatggatgtattttttcgtgccactgaactttcgttgggaagattagatacctctccctccccccccctccctttcaacacttcaaaacctaccgaaaactaccgatacaatttgatgtacctaccgaagaccgccaaaataatctggccacactggctagatctgatgttttgaaacatcgccaaattttactaaataatgcacttgcgcgttggtgaagctgcacttaaacagattagttgaattacgctttctgaaatgtttttattaaacacttgttttttttaatacttcaatttaaagctacttcttggaatgataattctacccaaaaccaaaaccactttacaaagagtgtgtggttggtgatcgcgccggctactgcgacctgtggtcatcgctcatctcacgcacacatacgcacacatctgtcatttgaaaagctgcacacaatcgtgtgttgtgcaaaaaaccgttcgcgcacaaacagttcgcacatgtttgcaagccgtgcagcaaagggcttttgatggtgatccgcagtgtttggtcagctttttaatgtgttttcgcttaatcgcttttgtttgtgtaatgttttgtcggaataatttttatgcgtcgttacaaatgaaatgtacaatcctgcgatgctagtgtaaaacattgctaattcccggtaagaacgttcattacgctcggatatcatcttacgaagcgtgtgtatgtgcgctagtacattgacctttaaagtgatccttttgttctagaaagcaag

SEQ ID NO: 9 - DNA, 849 bp, Artificial Sequence
  exu promoter
ggaaggtgattgcgattccatgttgatgccaatatatgatgattttgttgcatattaatagttgttgttatgttttattcaaatttcaaagataatttactttacattacagttagtgagcatattatctactacataaacacatagatcaaactggtttacataaattcaaaaagtttggattaaaatcgcagcaattggttatgaaaaaatatgtgcataacgtaaatatcaagtaaatttttgcattgcatatttatagactcctgttacaatttcggaaaaatgaaaaatgttaattaatcaaagaagaaaaaacaaagaaattaaatcattaggtagcacaaccacaagtacatatttttatggcatgaatattcctctacactaacatattttatagcaattctattgatcgccttagtatagcggaattaccagaacggcactatagttgtctctgtttggcacacgcaatcatttttcatcccagggttgccatagcagtttggcgacggtcacgtagcatgcgaaggatttcgttcgcacaggatcacttttattctaacgtttgaagaaggcacatctcagtgcaagcgctctggaagctgcttttaccgaacgaactaacttttcaagtaacctcaaaaacttgtctctaacgacaccacgtgctatccgcgagtttcatttcccgtgcaaagttccccgatttagctatcattcgtgaacatttcgtagtgcctctaccctcaggtaagaccattcgaggtttaccaagttttgtgcaaagaacgtgcacagtaattttcgttctggtgaaaccttctcttgtgtagcttgtacaaa

SEQ ID NO: 10 - DNA, 2291 bp, Artificial Sequence
  Vasa2
atgtagaacgcgagcaaattcttttccttccatgacagcagcagctacagtgggaagccgaacgtcagacgtgtttgacatgccgaactgggcgggaaaattacagcgtgcgctttgttttcaagcaaatcacaactcgctgcaaacaaaaccgttgagaaattgattgttttataatttgtattgtattttatttgttataataaactaaaaagacatactttttgcatattttatacataaaaacatacatgcagcattataaaacacatataaaccctccctgtagagtcccgtatcgaaatcttccatcctagttgcacagtacgacggacgagtaggccgtgtccgtgcaaattccagcttttagcagtcttttgctcggagcactcgcggcgagtcggaggtttctgctgaggtgcttagcgctaaattagccaattgcttttgcaagtgaaataaccagccgaatagtacttcaaaactcaggtaagtgaactagttttatagaacaaatgtttgtttgttagaagttagtgaagtgtttgtgaaaaaaatctctcatttcggcaaaactaacgtaactgatttcaaattgaattattgttttgtgatgttatattatttcatccagttgattagtattttcttagttatgttcaaaatacagttaaattaaatttcatttcatttactcataaaataatctcttggcttatttaatttttctcgaattcgcttgtattgttcagtagcacgcgccattcgccctttgtttcattttgtacctgctcccactaacacactggcagtgcgaaacaaaagccttcgcacgcgttgctggtattagagtgtgtgcgtgtgtgtgttgagcgctctgtcaaaatcggctgttgccgccggtaccgaaattgcctgttcgcacgctgttcgtaaacattccgtggtgtgtatcgtgtgttgtgcatgttgcgcgcctccccccttttgatagcaggctgccgtggctgccgtggtgtgtggcgcagttgagtttttggattaattttctaaggaaatggcacgagaagagcggtggcagtgtgttggtttgctctgtcccttcctttctgtgtgaagtgttcttacagcacagcacgtatccaccaccgcacacagagcaggcaaggaagtggaagtgaacaagtgtgctgcgcatgcatgtgtgtggggggcattttagctgagatcgtcgttatttgagaagcggtataggggccagtcggtgtcgacgtacggaagcggtttagttttaatccaagcgtatcccgtcgtggagtggttgtgtggctctgtgtgctctcatatcagttccagagtgaggttagtagaatcacagtccttggcctttttcgttacaagatatccagaaggatggcgttatttccacagcttaccatggtgctcttgtttgctcgaatcaggggagaaaaacagtttcgtgtttcatgaaccgcagttggcactggagcggattcaaaagtcttcgatatgcaatagataagagagtcgttggggcatagttgggaagcctttccgagatgtggagtttccgagaggagaaatggtgctttcgtgcacgttccgggacagcgggccccgcgaagagcatctcgttgtcgttcatccggcaataattgatgcgaaaagcgcgcgcgccactggcttagcgcagtgtacacagtgatattcacctacacacacagaggcacacgccttcacacgcgcgcgtgcttcaaaggctacttcggtggcggtgtgtgaggtcgcttgcaatggacaatgaaaatttcgctggaaaataccatcgtctctttaggttgcaatgggtgcgggtagagcggtggtcgtcgatattggtggtgtagtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgcaacggcaattattttttgtaatatttcgaccatctttctttctctctctccacgtgctgctgctgttgctgctgctgctgcattgcatgttccactattcctctcggtttgtgcctgcggacgccattgctagtcgaaagagagtcgccgttagtcgcgcttcgagcaacggacacgttttttggttgaaaccaacagcttttttcatcttcgggagacacacagatctcgaatcgtacattcccataaggagaattgtcatcttccggtgaataaagaaaggaaac

SEQ ID NO: 11 - DNA, 1885 bp, Artificial Sequence
  5′ homology arm
cttgtgtttagcaggcaggggagatgagcgcaaactgtgcaagaagaagcatcactgtgaagacggcaatgcaaagatagtgtgctcaacttctccgcgaagattgaagctaaattaagcacgagattagcatgactgaagtgacttttcaaagtgtcagaatggctgcactcgcaaactagctggatgcagcgcaattttgccccggtgtgtgcgcgcatgcaaacgagcaaccgcagagggcaaaggagaggatgggaaggagggagggagtgaaagagcaggcttaaggttgccctcgggcattgaagtcgatacagcggttctattccagtgccagtaacgatgacgaagacgatgttgcttctgctgctgttgctgctgttgttgttgatgatgatgatgataatagtgcaaatataaaataaatcttccgtaagctttgtgtagtggtgcgtggctactataagcccgtctggaagcaaggaagctagtcgggcagggtcatgcaaaagggagacaccttcggagctccggagctcccgccggcactctcggggggacgtccgttatgcgttgtgatttattatggaatatttattatagtgtcttgttttgaaaaaataacttcaacggttcgaatttcctacacctcgagatcggggctggagtggcaacgtggtacggaacggtacagcggtttgagccgttcggtcttgggactcacggatcgcagaatgttattgtgcgcgcactgatgggaaagtcatttttcaccgagtggtcagggcgcgtagtccagttcgtttctggctgctgttgctgatgctacgatcctcaggaatgattggaaacgcctggagatggtgggaaaaaatcaaacacaaaaacgatcctaatgaacatcgtgtgttctcattcgctgccacgattgacaccttcgataagacgcacataatgagctaaaggagaggggacagggtcttgtctttgccacgagcgataagattgcaatcactcgtgagcgtgtgctgctgggctgaagaagaaacgctttccacagcagtaggtgggaagtgggattgtggaacgtggcattgaaaagaacctattttctaaagcccgagagcccgttctcgaactggaaaaccagatgcagaagttttttattgtcccccgccaggaaaacaaatgtatttaatgctttctttgccttttccgccccgtttcagacgacgagctagtgaagcgagcccaatggctgttggagaaactcggctacccgtgggagatgatgcccctgatgtacgtcatactaaagagcgccgatggcgatgtacaaaaagcacaccagcggatcgacgaaggtaagctggcgatgatggtgtcgttcgacatcactttcatcaccgtgtcagacatctactgtgcctagcaccgggtccagtggtcacagggtgtagcaaaaacgtgttcttttttgcgagagactctacctcatgatgcagctgttaaggaaaggtttcagatgaaggcaatttttcctaggataagatgatcttaagttacctgcgtattagtgtttaacattgtcgtctcaactcccaagaatgttttaatcgtctagggctagtttatttatactgttctcattgaaatgtcgttcaatccaacatgttaagttagctagctcagacacgagaagttaggagtatctgcatcttgaaggtagcggcatatggtgttatgccacgttcactgacttcaaaattcgatacaaaaaaaaaaccaaaacatcaaaaaccaaattgtgaattccgtcagccagcagcagtgaccttcaaagccttacctttccattcatttatgtttaacacaggtcaag

SEQ ID NO: 12 - DNA, 1961 bp, Artificial Sequence
  3′ homology arm
cggtggtcaacgaatactcacgattgcataatctgaacatgtttgatggcgtggagttgcgcaataccacccgtcagagtggatgataaactttccgcaccactgtaactgtccgtatctttgtatgtgggtgtgtgtatgtgtgtttggtgaaacgaattcaatagttctgtgctattttaaatcaagccgcgtgcgcaactgatgccgataagttcaaactagtgtttaaggagtggagcgagagagccgcaccacggtacagaagggcagcagaatgggtcggcagcctagctgcactggtgcggtgcgtccggcgtctcggggggagggcgaggaaattctagtgttaaatcggagcagcaaaaacaaaacagtggtcgtcccgttcaagaaacggcctgtacacacacacagaaaacactgcagcatgtttgtacatagtagatcctagagcaggtggtcgttgctcctcgaacgctctggacgcacggcttcgcgcgtatttgcgtagcgttccgccgatcgtgggtattcgtactgccacaagcccgctttctcccatgcaatctctgcaaccaaaccaacaaacaacaacaaaaaaccaatcgacaaaatgaatcacaccccttttgtatcatctgtatattcttgttctttgcgttcttttctatgtggcccacgccccggcgggtacgtaattgcgtcgaaaaccccgaaaaccccggcacatacagtgtacatacggtttgaggacaactttgacctgcagcccttctggggttgccacgtgtagctatacttgtgagatcgggcgccgacggtgtaaagcgcgaatggccgccacacagtgtgtccactccaacactacccctctggaactaccccgtccagggatgcaccggctcggctcatgcccctgcaaaacagtccgggctccactgtagtagctccggcgttgctctgagagaaggatgcccttcgaagtgtcgaaagcgtgcattgggcgttcaagtgtgtgtgtgtgtgttaggtttagcgagaaacagcagcagttgcgtgtgctgaaaagcgaaggagtaatagagtgcataatgaaaatgaaaatgaaaatgaagcaaaagtagaaggcggaggagagcaacctgtgttccactagtagcgaatagtttagtctagtttcgtcaccaatcaaccttccaaccatcgttcaaccaatacctgagtcaacatcgtcatcgttatcgtgccacaactttattaaaaatgaaccttgtccgcgccaccgtagggtgatctaaggcgacctttcttacgggcgcgacccacatgccatcgtcaccttctccaatcaaaaccaacagcctgtaccgatggtgtgcaattgtgcgtgcgtgtgtgttattagcaaaaaaagagaaagagtcgacgagagagagatagatcgagatcgagagtacaaaagagcagtagaaatgttcgttgtttgtttttcgtaacacagttgtttagccaaaatgggaatttccaataatcccgggggcggggaaatgcgggaatactgcgtacacacatacatcaatcaaaaagaaaaatccttgcgctacatcactaccgtttgcgcggtgctgatctagagcagaccactttccactccactctacaatcaatcaatctgtgcagaaggtatggtaagacggcctttgagcgagtcacggtcgccaccataacgccgtccgacgagggctgaatgcgaactttgctaatcgattttccgctttctttttatcccacctccttttctctccctctctctcttttgcactgccccttgtaacccccaaaaaggtaaacgacacattaagacctacgaagcgttggtgaagtcatcgctcgatccgaacagcgaccggctgacggaggacgacgacgaggacgagaacatctcggtgacccgcacc

SEQ ID NO: 13 - DNA, 8005 bp, Artificial Sequence
  Gene Drive construct
tgcgggtgccagggcgtgcccttgggctccccgggcgcgtactccacctcacccatgcgatcgctccggaaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctagagtcgcggccgctacaggaacaggtggtggcggccctcggtgcgctcgtactgctccacgatggtgtagtcctcgttgtgggaggtgatgtccagcttggagtccacgtagtagtagccgggcagctgcacgggcttcttggccatgtagatggacttgaactccaccaggtagtggccgccgtccttcagcttcagggccttgtggatctcgcccttcagcacgccgtcgcgggggtacaggcgctcggtggaggcctcccagcccatggtcttcttctgcattacggggccgtcggaggggaagttcacgccgatgaacttcaccttgtagatgaagcagccgtcctgcagggaggagtcttgggtcacggtcaccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactccttgatgacgttcttggaggagcgcaccatggtggcgacctgtgggtcccgggcccgcggtaccgtcgactctagcggtaccccgattgtttagcttgttcagctgcgcttgtttatttgcttagctttcgcttagcgacgtgttcactttgcttgtttgaattgaattgtcgctccgtagacgaagcgcctctatttatactccggcggtcgagggttcgaaatcgataagcttggatcctaattgaattagctctaattgaattagtctctaattgaattagatccccgggcgagctcgaattaaccattgtggaccggtcagcgctggcggtggggacagctccggctgtggctgttcttgagagtcatcttcctgcggcacatccctctcgtcgaccagttcagtttgctgagcgtaagcctgctgctgttcgtcctgcatcatcgggaccatttgtacgggccatccgccaccaccaccatcaccaccgccgtccatttctaggggcatacccatcagcatctccgcgggcgccattggcggtggtgccaaggtgccattcgtttgttgctgaaagcaaaagaaagcaaattagtgttgtttctgctgcacacgatagttttcgtttcttgccgctagacacaaacaacactgcatctggagggagaaatttgacgcctagctgtataacttacctcaaagttattgtccatcgtggtataatggacctaccgagcccggttacactacacaaagcaagattatgcgacaaaatcacagcgaaaactagtaattttcatctatcgaaagcggccgagcagagagttgtttggtattgcaacttgacattctgctgtgggataaaccgcgacgggctaccatggcgcacctgtcagatggctgtcaaatttggcccggtttgcgatatggagtgggtgaaattatatcccactcgctgatcgtgaaaatagacacctgaaaacaataattgttgtgttaattttacattttgaagaacagcacaagttttgctgacaatatttaattacgtttcgttatcaacggcacggaaagattatctcgctgattatccctctcgctctctctgtctatcatgtcctggtcgttctcgcgtcaccccggataatcgagagacgccatttttaatttgaactactacaccgacaagcatgccgtgagctctttcaagttcttctgtccgaccaaagaaacagagaataccgcccggacagtgcccggagtgatcgatccatagaaaatcgcccatcatgtgccactgaagcgaaccggcgtagcttgttccgaatttccaagtgcttccccgtaacatccgcatataacaagcagcccaacaacaaatacagcatcgagctcgagatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaattaattaagaggacggcgagaagtaatcatatgtccgcattttgcgcaaaccaggcgcttagacaatttgcgcgtaagcacattcgaaatgtgaaaagctgaaagcagtggtttcgccagcccgagttcagcgaaacggattccttccaagtgtttgcattcctggcggagtgttcctcccaaaatgcactcaccctgcgtgcagtgccaaatcgtgagtttcctaattttttcatattgtttattacctaccaactaaagttgttgttatatattgcgttttacgtacgacaaataagttcgtattcagaaatatttgcgataagagagaactcatttgcgatgaatctcattgtatttagctaagtgccttgataagtaagcggaacagcaggaatatgacactccttgggaaatacatgtaagcgtctgtaattagatatatatacacgcaaccaaatggtccatggttgatttaagcactgcctgttgtcgaacattgctataagcaaaataaagaagcattcattaatctaaaatttcttcaaagtgacttcaatgatgatctctaggctatagtgaaagctgaaagcttatttgacaatgcaagggaaagtgacgcacgtgcgtcgtatgggaccgcgcgcatctattctctcagctaattcccctaatcattagtaattgacggcacgatttctgcttcttacttccttttactttggagcttttcatcaataaaaccagtaccatggccgtacgctcaacggaaaagcattcaaaaaaacccgcgttcctcgtgtgatttgtgggtgagtggcgccatctattagagaatagctgtactacatctcgtggacgaaggggtcagagaagttgaaagagagcttgatcgactgctatccaagctaggcgaggaagggagatcgctagagcaaaagaaaaaaaataagcaaatatctttttttataacaaatcgacgttagcgaaatatgtttgaatcgatttaacggttagaattccctttggttcgttcattatgcgaggcgcgcctttgtatgcgtgcgcttgaagggttgatcggaaccttacaacagttgtagctatacggctgcgtgtggcttctaacgttatccatcgctagaagtgaaacgaatgtgcgtaggtatatatatgaaatggagttgctctctgctgtttaacacaggtcaagcgggttttagagctagaaatagcaagttaaaataaggctagtccgttatcaacttgaaaaagtggcaccgagtcggtgcttttttttacgcgtgggtcccatgggtgaggtggagtacgcgcccggggagcccaagggcacgccctggcacccgca

SEQ ID NO: 14 - DNA, 24 bp, Artificial Sequence
  dsxgRNA-F primer
tgctgtttaacacaggtcaagcgg

SEQ ID NO: 15 - DNA, 24 bp, Artificial Sequence
  dsxgRNA-R primer
aaacccgcttgacctgtgttaaac

SEQ ID NO: 16 - DNA, 48 bp, Artificial Sequence
  dsx?31L-F primer
gctcgaattaaccattgtggaccggtcttgtgtttagcaggcagggga

SEQ ID NO: 17 - DNA, 49 bp, Artificial Sequence
  dsx?31L-R primer
tccacctcacccatgggacccacgcgtggtgcgggtcaccgagatgttc

SEQ ID NO: 18 - DNA, 50 bp, Artificial Sequence
  dsx?31R-F primer
caccaagacagttaacgtatccgttaccttgacctgtgttaaacataaat

SEQ ID NO: 19 - DNA, 49 bp, Artificial Sequence
  dsx?31R-R primer
ggtggtagtgccacacagagagcttcgcggtggtcaacgaatactcacg

SEQ ID NO: 20 - DNA, 44 bp, Artificial Sequence
  zpgprCRISPR-F primer
gctcgaattaaccattgtggaccggtcagcgctggcggtgggga

SEQ ID NO: 21 - DNA, 46 bp, Artificial Sequence
  zpgprCRISPR-R primer
tcgtggtccttatagtccatctcgagctcgatgctgtatttgttgt

SEQ ID NO: 22 - DNA, 50 bp, Artificial Sequence
  zpgteCRISPR-F primer
aggcaaaaaagaaaaagtaattaattaagaggacggcgagaagtaatcat

SEQ ID NO: 23 - DNA, 51 bp, Artificial Sequence
  zpgteCRISPR-R primer
ttcaagcgcacgcatacaaaggcgcgcctcgcataatgaacgaaccaaagg

SEQ ID NO: 24 - DNA, 20 bp, Artificial Sequence
  dsxin3-F primer
ggcccttcaacccgaagaat

SEQ ID NO: 25 - DNA, 20 bp, Artificial Sequence
  dsxex6-R primer
ctttttgtacagcggtacac

SEQ ID NO: 26 - DNA, 20 bp, Artificial Sequence
  GFP-F primer
gccctgagcaaagaccccaa

SEQ ID NO: 27 - DNA, 22 bp, Artificial Sequence
  dsxex4-F primer
gcacaccagcggatcgacgaag

SEQ ID NO: 28 - DNA, 23 bp, Artificial Sequence
  dsxex5-R primer
cccacatacaaagatacggacag

SEQ ID NO: 29 - DNA, 22 bp, Artificial Sequence
  dsxex6-R primer
gaatttggtgtcaaggttcagg

SEQ ID NO: 30 - DNA, 22 bp, Artificial Sequence
  3xP3 primer
tatactccggcggtcgagggtt

SEQ ID NO: 31 - DNA, 22 bp, Artificial Sequence
  hCas9-F primer
ccaagagagtgatcctggccga

SEQ ID NO: 32 - DNA, 22 bp, Artificial Sequence
  dsxex5-R1 primer
cttatcggcatcagttgcgcac

SEQ ID NO: 33 - DNA, 22 bp, Artificial Sequence
  dsxin4-F primer
ggtgttatgccacgttcactga

SEQ ID NO: 34 - DNA, 22 bp, Artificial Sequence
  RFP-R primer
caagtgggagcgcgtgatgaac

SEQ ID NO: 35 - DNA, 1712 bp, Unknown
  nucleotide sequence of exon 5 of the doublesex (dsx) gene
gtcaagcggtggtcaacgaatactcacgattgcataatctgaacatgtttgatggcgtggagttgcgcaataccacccgtcagagtggatgataaactttccgcaccactgtaactgtccgtatctttgtatgtgggtgtgtgtatgtgtgtttggtgaaacgaattcaatagttctgtgctattttaaatcaagccgcgtgcgcaactgatgccgataagttcaaactagtgtttaaggagtggagcgagagagccgcaccacggtacagaagggcagcagaatgggtcggcagcctagctgcactggtgcggtgcgtccggcgtctcggggggagggcgaggaaattctagtgttaaatcggagcagcaaaaacaaaacagtggtcgtcccgttcaagaaacggcctgtacacacacacagaaaacactgcagcatgtttgtacatagtagatcctagagcaggtggtcgttgctcctcgaacgctctggacgcacggcttcgcgcgtatttgcgtagcgttccgccgatcgtgggtattcgtactgccacaagcccgctttctcccatgcaatctctgcaaccaaaccaacaaacaacaacaaaaaaccaatcgacaaaatgaatcacaccccttttgtatcatctgtatattcttgttctttgcgttcttttctatgtggcccacgccccggcgggtacgtaattgcgtcgaaaaccccgaaaaccccggcacatacagtgtacatacggtttgaggacaactttgacctgcagcccttctggggttgccacgtgtagctatacttgtgagatcgggcgccgacggtgtaaagcgcgaatggccgccacacagtgtgtccactccaacactacccctctggaactaccccgtccagggatgcaccggctcggctcatgcccctgcaaaacagtccgggctccactgtagtagctccggcgttgctctgagagaaggatgcccttcgaagtgtcgaaagcgtgcattgggcgttcaagtgtgtgtgtgtgtgttaggtttagcgagaaacagcagcagttgcgtgtgctgaaaagcgaaggagtaatagagtgcataatgaaaatgaaaatgaaaatgaagcaaaagtagaaggcggaggagagcaacctgtgttccactagtagcgaatagtttagtctagtttcgtcaccaatcaaccttccaaccatcgttcaaccaatacctgagtcaacatcgtcatcgttatcgtgccacaactttattaaaaatgaaccttgtccgcgccaccgtagggtgatctaaggcgacctttcttacgggcgcgacccacatgccatcgtcaccttctccaatcaaaaccaacagcctgtaccgatggtgtgcaattgtgcgtgcgtgtgtgttattagcaaaaaaagagaaagagtcgacgagagagagatagatcgagatcgagagtacaaaagagcagtagaaatgttcgttgtttgtttttcgtaacacagttgtttagccaaaatgggaatttccaataatcccgggggcggggaaatgcgggaatactgcgtacacacatacatcaatcaaaaagaaaaatccttgcgctacatcactaccgtttgcgcggtgctgatctagagcagaccactttccactccactctacaatcaatcaatctgtgcagaaggtatggtaagacggcctttg

SEQ ID NO: 36 - DNA, 23 bp, Unknown
  T2 target site
tctgaacatgtttgatggcgtgg

SEQ ID NO: 37 - DNA, 22 bp, Unknown
  T3 target site
gcaataccacccgtcagagtgg

SEQ ID NO: 38 - DNA, 21 bp, Unknown
  T4 target site
gtttatcatccactctgacgg

SEQ ID NO: 39 - DNA, 20 bp, Artificial Sequence
  nucleotide sequence encoding a nucleotide sequence that is capable of hybridising to T2
tctgaacatgtttgatggcg

SEQ ID NO: 40 - DNA, 19 bp, Artificial Sequence
  nucleotide sequence encoding a nucleotide sequence that is capable of hybridising to T3
gcaataccacccgtcagag

SEQ ID NO: 41 - DNA, 18 bp, Artificial Sequence
  nucleotide sequence encoding a nucleotide sequence that is capable of hybridising to T4
gtttatcatccactctga

SEQ ID NO: 42 - DNA, 97 bp, Artificial Sequence
  nd nucleotide sequence encoding a nucleotide sequence that is capable of hybridising to the second target site
tctgaacatgtttgatggcggttttagagctagaaatagcaagttaaaataaggctagtccgttatcaacttgaaaaagtggcaccgagtcggtgct

SEQ ID NO: 43 - DNA, 96 bp, Artificial Sequence
  second nucleotide sequence encoding a nucleotide sequence that is capable of hybridising to T3
gcaataccacccgtcagaggttttagagctagaaatagcaagttaaaataaggctagtccgttatcaacttgaaaaagtggcaccgagtcggtgct

SEQ ID NO: 44 - DNA, 95 bp, Artificial Sequence
  second nucleotide sequence encoding a nucleotide sequence that is capable of hybridising to T4
gtttatcatccactctgagttttagagctagaaatagcaagttaaaataaggctagtccgttatcaacttgaaaaagtggcaccgagtcggtgct

SEQ ID NO: 45 - RNA, 97 nt, Artificial Sequence
  second guide RNA targeting T2
ucugaacauguuugauggcgguuuuagagcuagaaauagcaaguuaaaauaaggcuaguccguuaucaacuugaaaaaguggcaccgagucggugcu

SEQ ID NO: 46 - RNA, 96 nt, Artificial Sequence
  second guide RNA targeting T3
gcaauaccacccgucagagguuuuagagcuagaaauagcaaguuaaaauaaggcuaguccguuaucaacuugaaaaaguggcaccgagucggugcu

SEQ ID NO: 47 - RNA, 95 nt, Artificial Sequence
  second guide RNA targeting T4
guuuaucauccacucugaguuuuagagcuagaaauagcaaguuaaaauaaggcuaguccguuaucaacuugaaaaaguggcaccgagucggugcu

SEQ ID NO: 48 - RNA, 97 nt, Artificial Sequence
  guide RNA to dsx
guuuaacacaggucaagcggguuuuagagcuagaaauagcaaguuaaaauaaggcuaguccguuaucaacuugaaaaaguggcaccgagucggugcu

SEQ ID NO: 49 - DNA, 143 bp, Artificial Sequence
  U6 promoter
tttgtatgcgtgcgcttgaagggttgatcggaaccttacaacagttgtagctatacggctgcgtgtggcttctaacgttatccatcgctagaagtgaaacgaatgtgcgtaggtatatatatgaaatggagttgctctctgct

SEQ ID NO: 50 - DNA, 1885 bp, Artificial Sequence
  5′ homology arm
gagtggatgataaactttccgcaccactgtaactgtccgtatctttgtatgtgggtgtgtgtatgtgtgtttggtgaaacgaattcaatagttctgtgctattttaaatcaagccgcgtgcgcaactgatgccgataagttcaaactagtgtttaaggagtggagcgagagagccgcaccacggtacagaagggcagcagaatgggtcggcagcctagctgcactggtgcggtgcgtccggcgtctcggggggagggcgaggaaattctagtgttaaatcggagcagcaaaaacaaaacagtggtcgtcccgttcaagaaacggcctgtacacacacacagaaaacactgcagcatgtttgtacatagtagatcctagagcaggtggtcgttgctcctcgaacgctctggacgcacggcttcgcgcgtatttgcgtagcgttccgccgatcgtgggtattcgtactgccacaagcccgctttctcccatgcaatctctgcaaccaaaccaacaaacaacaacaaaaaaccaatcgacaaaatgaatcacaccccttttgtatcatctgtatattcttgttctttgcgttcttttctatgtggcccacgccccggcgggtacgtaattgcgtcgaaaaccccgaaaaccccggcacatacagtgtacatacggtttgaggacaactttgacctgcagcccttctggggttgccacgtgtagctatacttgtgagatcgggcgccgacggtgtaaagcgcgaatggccgccacacagtgtgtccactccaacactacccctctggaactaccccgtccagggatgcaccggctcggctcatgcccctgcaaaacagtccgggctccactgtagtagctccggcgttgctctgagagaaggatgcccttcgaagtgtcgaaagcgtgcattgggcgttcaagtgtgtgtgtgtgtgttaggtttagcgagaaacagcagcagttgcgtgtgctgaaaagcgaaggagtaatagagtgcataatgaaaatgaaaatgaaaatgaagcaaaagtagaaggcggaggagagcaacctgtgttccactagtagcgaatagtttagtctagtttcgtcaccaatcaaccttccaaccatcgttcaaccaatacctgagtcaacatcgtcatcgttatcgtgccacaactttattaaaaatgaaccttgtccgcgccaccgtagggtgatctaaggcgacctttcttacgggcgcgacccacatgccatcgtcaccttctccaatcaaaaccaacagcctgtaccgatggtgtgcaattgtgcgtgcgtgtgtgttattagcaaaaaaagagaaagagtcgacgagagagagatagatcgagatcgagagtacaaaagagcagtagaaatgttcgttgtttgtttttcgtaacacagttgtttagccaaaatgggaatttccaataatcccgggggcggggaaatgcgggaatactgcgtacacacatacatcaatcaaaaagaaaaatccttgcgctacatcactaccgtttgcgcggtgctgatctagagcagaccactttccactccactctacaatcaatcaatctgtgcagaaggtatggtaagacggcctttgagcgagtcacggtcgccaccataacgccgtccgacgagggctgaatgcgaactttgctaatcgattttccgctttctttttatcccacctccttttctctccctctctctcttttgcactgccccttgtaacccccaaaaaggtaaacgacacattaagacctacgaagcgttggtgaagtcatcgctcgatccgaacagcgaccggctgacggaggacgacgacgaggacgagaacatctcggtgacccgcacc

SEQ ID NO: 51 - DNA, 8251 bp, Artificial Sequence
  multiplex CRISPR construct
tgcgggtgccagggcgtgcccttgggctccccgggcgcgtactccacctcacccatgcgatcgctccggaaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctagagtcgcggccgctacaggaacaggtggtggcggccctcggtgcgctcgtactgctccacgatggtgtagtcctcgttgtgggaggtgatgtccagcttggagtccacgtagtagtagccgggcagctgcacgggcttcttggccatgtagatggacttgaactccaccaggtagtggccgccgtccttcagcttcagggccttgtggatctcgcccttcagcacgccgtcgcgggggtacaggcgctcggtggaggcctcccagcccatggtcttcttctgcattacggggccgtcggaggggaagttcacgccgatgaacttcaccttgtagatgaagcagccgtcctgcagggaggagtcttgggtcacggtcaccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactccttgatgacgttcttggaggagcgcaccatggtggcgacctgtgggtcccgggcccgcggtaccgtcgactctagcggtaccccgattgtttagcttgttcagctgcgcttgtttatttgcttagctttcgcttagcgacgtgttcactttgcttgtttgaattgaattgtcgctccgtagacgaagcgcctctatttatactccggcggtcgagggttcgaaatcgataagcttggatcctaattgaattagctctaattgaattagtctctaattgaattagatccccgggcgagctcgaattaaccattgtggaccggtcagcgctggcggtggggacagctccggctgtggctgttcttgagagtcatcttcctgcggcacatccctctcgtcgaccagttcagtttgctgagcgtaagcctgctgctgttcgtcctgcatcatcgggaccatttgtacgggccatccgccaccaccaccatcaccaccgccgtccatttctaggggcatacccatcagcatctccgcgggcgccattggcggtggtgccaaggtgccattcgtttgttgctgaaagcaaaagaaagcaaattagtgttgtttctgctgcacacgatagttttcgtttcttgccgctagacacaaacaacactgcatctggagggagaaatttgacgcctagctgtataacttacctcaaagttattgtccatcgtggtataatggacctaccgagcccggttacactacacaaagcaagattatgcgacaaaatcacagcgaaaactagtaattttcatctatcgaaagcggccgagcagagagttgtttggtattgcaacttgacattctgctgtgggataaaccgcgacgggctaccatggcgcacctgtcagatggctgtcaaatttggcccggtttgcgatatggagtgggtgaaattatatcccactcgctgatcgtgaaaatagacacctgaaaacaataattgttgtgttaattttacattttgaagaacagcacaagttttgctgacaatatttaattacgtttcgttatcaacggcacggaaagattatctcgctgattatccctctcgctctctctgtctatcatgtcctggtcgttctcgcgtcaccccggataatcgagagacgccatttttaatttgaactactacaccgacaagcatgccgtgagctctttcaagttcttctgtccgaccaaagaaacagagaataccgcccggacagtgcccggagtgatcgatccatagaaaatcgcccatcatgtgccactgaagcgaaccggcgtagcttgttccgaatttccaagtgcttccccgtaacatccgcatataacaagcagcccaacaacaaatacagcatcgagctcgagatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaattaattaagaggacggcgagaagtaatcatatgtccgcattttgcgcaaaccaggcgcttagacaatttgcgcgtaagcacattcgaaatgtgaaaagctgaaagcagtggtttcgccagcccgagttcagcgaaacggattccttccaagtgtttgcattcctggcggagtgttcctcccaaaatgcactcaccctgcgtgcagtgccaaatcgtgagtttcctaattttttcatattgtttattacctaccaactaaagttgttgttatatattgcgttttacgtacgacaaataagttcgtattcagaaatatttgcgataagagagaactcatttgcgatgaatctcattgtatttagctaagtgccttgataagtaagcggaacagcaggaatatgacactccttgggaaatacatgtaagcgtctgtaattagatatatatacacgcaaccaaatggtccatggttgatttaagcactgcctgttgtcgaacattgctataagcaaaataaagaagcattcattaatctaaaatttcttcaaagtgacttcaatgatgatctctaggctatagtgaaagctgaaagcttatttgacaatgcaagggaaagtgacgcacgtgcgtcgtatgggaccgcgcgcatctattctctcagctaattcccctaatcattagtaattgacggcacgatttctgcttcttacttccttttactttggagcttttcatcaataaaaccagtaccatggccgtacgctcaacggaaaagcattcaaaaaaacccgcgttcctcgtgtgatttgtgggtgagtggcgccatctattagagaatagctgtactacatctcgtggacgaaggggtcagagaagttgaaagagagcttgatcgactgctatccaagctaggcgaggaagggagatcgctagagcaaaagaaaaaaaataagcaaatatctttttttataacaaatcgacgttagcgaaatatgtttgaatcgatttaacggttagaattccctttggttcgttcattatgcgaggcgcgcctttgtatgcgtgcgcttgaagggttgatcggaaccttacaacagttgtagctatacggctgcgtgtggcttctaacgttatccatcgctagaagtgaaacgaatgtgcgtaggtatatatatgaaatggagttgctctctgctgtttaacacaggtcaagcgggttttagagctagaaatagcaagttaaaataaggctagtccgttatcaacttgaaaaagtggcaccgagtcggtgctttttttttttgtatgcgtgcgcttgaagggttgatcggaaccttacaacagttgtagctatacggctgcgtgtggcttctaacgttatccatcgctagaagtgaaacgaatgtgcgtaggtatatatatgaaatggagttgctctctgctgcaataccacccgtcagaggttttagagctagaaatagcaagttaaaataaggctagtccgttatcaacttgaaaaagtggcaccgagtcggtgcttttttttacgcgtgggtcccatgggtgaggtggagtacgcgcccggggagcccaagggcacgccctggcacccgca

SEQ ID NO: 52 - DNA, 48 bp, Artificial Sequence; identical to SEQ ID NO: 16
  multidsx?31L-F primer

SEQ ID NO: 53 - DNA, 44 bp, Artificial Sequence
  multidsx?31L-R primer
tgaacgattggggtaccggtcttgacctgtgttaaacataaatg

SEQ ID NO: 54 - DNA, 44 bp, Artificial Sequence
  multidsx?31R-F primer
agatataatcctgaacgcgtgagtggatgataaactttccgcac

SEQ ID NO: 55 - DNA, 49 bp, Artificial Sequence; identical to SEQ ID NO: 17
  multidsx?31R-R primer

SEQ ID NO: 56 - DNA, 58 bp, Artificial Sequence
  4050-2U6-T1-F primer
gagggtctcatgctgtttaacacaggtcaagcgggttttagagctagaaatagcaagt

SEQ ID NO: 57 - DNA, 56 bp, Artificial Sequence
  4050-2U6-T3-R primer
gagggtctcaaaacctctgacgggtggtattgcagcagagagcaactccatttcat

SEQ ID NO: 58 - RNA, 20 nt, Artificial Sequence
  guide RNA component
guuuaacacaggucaagcgg

SEQ ID NO: 59 - RNA, 20 nt, Artificial Sequence
  second guide RNA targeting T2 component
ucugaacauguuugauggcg

SEQ ID NO: 60 - RNA, 19 nt, Artificial Sequence
  second guide RNA targeting T3 component
gcaauaccacccgucagag

SEQ ID NO: 61 - RNA, 18 nt, Artificial Sequence
  second guide RNA targeting T4 component
guuuaucauccacucuga

SEQ ID NO: 62 - DNA, 30 bp, Unknown
  Intron 4 Exon 5 boundary
ttatgtttaacacaggtcaagcggtggtca

SEQ ID NO: 63 - DNA, 30 bp, Unknown
  Intron 4 exon 5 boundary
aatacaaattgtgtccagttcgccaccagt

SEQ ID NO: 64 - DNA, 54 bp, Unknown; identical to SEQ ID NO: 3
  intron 4 exon 5 boundary

SEQ ID NO: 65 - DNA, 37 bp, Unknown
  intron 4 exon 5 boundary
gtttaacacaggtcaagcggtggtcaacgaatactca

SEQ ID NO: 66 - DNA, 26 bp, Unknown
  Intron 4 exon 5 boundary
gtttaacacaggtcaacgaatactca

SEQ ID NO: 67 - DNA, 33 bp, Unknown
  intron 4 exon 5 boundary
gtttaacacaggtcggtggtcaacgaatactca

SEQ ID NO: 68 - DNA, 28 bp, Unknown
  intron 4 exon 5 boundary
gtttaacacggtggtcaacgaatactca

SEQ ID NO: 69 - DNA, 26 bp, Unknown
  intron 4 exon 5 boundary
gtttaacggtggtcaacgaatactca

SEQ ID NO: 70 - DNA, 36 bp, Unknown
  intron 4 exon 4 boundary
gtttaacacaggtcaacggtggtcaacgaatactca

SEQ ID NO: 71 - DNA, 34 bp, Unknown
  intron 4 exon 5 boundary
gtttaacacaggtccggtggtcaacgaatactca

SEQ ID NO: 72 - DNA, 29 bp, Unknown
  intron 4 exon 5 boundary
gtttaacaccggtggtcaacgaatactca

SEQ ID NO: 73 - DNA, 27 bp, Unknown
  intron 4 exon 5 boundary
gtttaaccggtggtcaacgaatactca

SEQ ID NO: 74 - DNA, 39 bp, Unknown
  intron 4 exon 5 boundary
gtttaacacaggtcataagcggtggtcaacgaatactca

SEQ ID NO: 75 - DNA, 39 bp, Unknown
  Intron 4 exon 5 boundary
gtttaacacaggtcaaggacggtggtcaacgaatactca

SEQ ID NO: 76 - DNA, 129 bp, Unknown; identical to SEQ ID NO: 2
  Intron 4 exon 5 boundary

SEQ ID NO: 77 - DNA, 129 bp, Unknown; identical to SEQ ID NO: 2
  intron 4 exon 5 boundary

SEQ ID NO: 78 - DNA, 129 bp, Unknown; identical to SEQ ID NO: 2
  intron 4 exon 5 boundary

SEQ ID NO: 79 - DNA, 129 bp, Unknown; identical to SEQ ID NO: 2
  intron 4 exon 5 boundary

SEQ ID NO: 80 - DNA, 129 bp, Unknown; identical to SEQ ID NO: 2
  intron 4 exon 5 boundary

SEQ ID NO: 81 - DNA, 129 bp, Unknown
  SEQ ID No 82
cctttccattcatttatgtttaacacaggtcaagcggtggtcaacgaatactcacgattgcataatctgaacatgtttgatggcgtggagttgcgtaataccacccgtcagagtggatgataaactttc

SEQ ID NO: 82 - DNA, 129 bp, Unknown
  intron 4 exon 5 boundary
cctttccattcatttatgtttaacacaggtcaagcggtggtcaacgaatactcacgattgcataatctgaacatgttcgatggcgtggagttgcgcaataccacccgtcagagtggatgataaactttc

SEQ ID NO: 83 - DNA, 129 bp, Unknown
  intron 4 exon 5 boundary
cctttccattcatttatgttcaacacaggtcaagcggtggtcaacgaatactcacgattgcataatctgaacatgttcgatggcgtggagttgcgcaataccacccgtcagagtggatgataaactttc

SEQ ID NO: 84 - DNA, 129 bp, Unknown
  intron 4 exon 5 boundary
cctttccattcatttatgttcaacacaggtcaaacggtggtcaacgaatactcacgattgcataatctgaacatgttcgatggcgtggagttacgcaataccacccgtcagagtggatgataaactttc

SEQ ID NO: 85 - DNA, 128 bp, Unknown
  intron 4 exon 5 boundary
cctttccattcatttatgttcaacacaggtcaaacggtggtcaacgaatactcacgattgcataatctgaacatgttcgatggcgtggagttacgcaataccacccgtcagagtggatgataaacttt

SEQ ID NO: 86 - DNA, 129 bp, Unknown
  Intron 4 exon 5 boundary
cctttccattcatttatgttcaacacaggtcaagcggtggtcaacgaatactcaagattgcataatctgaacatgttcgatggcgtggagttacgcaataccacccgtcagagtggatgataaactttc

SEQ ID NO: 87 - DNA, 129 bp, Unknown
  intron 4 exon 5 boundary
cctttccattcatttatgttcaacacaggtcaagcggtggtcaacgaatactcacgattgcataatctgaacatgttcgatggcgtggagttacgcaataccacccgtcagagtggatgataaactttc

SEQ ID NO: 88 - DNA, 129 bp, Unknown
  intron 4 exon 5 boundary
ccttaccatgcatttatgttcaacacaggtcaagcggtggtcaacgaatactcacgattgcataatctgaacatgttcgatggcgtggagttacgcaacaccacccgtcagagtggatgataaactttc

SEQ ID NO: 89 - DNA, 129 bp, Unknown
  intron 4 exon 5 boundary
cctttccattcatttatgttcaacacaggtcaagcggtggtcaacgaatactcacgattgcataatctgaacatgttcgatggcgcggagttgcgcaataccacccgtcagagtggatgataaactttc

SEQ ID NO: 90 - DNA, 129 bp, Unknown; identical to SEQ ID NO: 89
  intron 4 exon 5 boundary

SEQ ID NO: 91 - DNA, 129 bp, Unknown
  intron 4 exon 5 boundary
cctttccattcatttatgctcaacacaggtcaggccgtggtcaacgaatactcacgattgcacaatctgaacatgttcgatggcgtggagttgcgcaacaccacccgtcagagtggatgataaactttc

SEQ ID NO: 92 - DNA, 129 bp, Unknown; identical to SEQ ID NO: 91
  intron 4 exon 5 boundary

SEQ ID NO: 93 - DNA, 129 bp, Unknown
  intron 4 exon 5 boundary
ctttgccatttatttatgcccaacacaggtcaggccgtggtcaacgaatactcacgattgcacaatctgaacatgttcgatggcgtagagttgcgcaacgccacccgccagagcggatgataaacttcc

SEQ ID NO: 94 - DNA, 129 bp, Unknown
  intron 4 exon 5 boundary
cctttccattcatttatgtttaacacaggtcaagcagtggtcaacgaatattcacgattgcataatctgaacatgtttgatggcgtggagttgcgcaataccacccgtcagagtggatgataaactttc